(19) United States
(12) Patent Application Publication
(10) Pub. No.: US 2026/0216250 A1
(43) Pub. Date: Jul. 30, 2026

(54) Compositions and Methods for Treating a Neurologic Disease Using Dopaminergic Neurons with Enhanced Anti-inflammatory Activity

(71) Applicant: Kenai Therapeutics Inc., Cambridge, MA (US)

(72) Inventor: Howard Federoff, Cambridge, MA (US)

(73) Assignee: Kenai Therapeutics Inc., Cambridge, MA (US)

(21) Appl. No.: 19/146,692

(22) PCT Filed: Jan. 10, 2024
(86) PCT No.: PCT/US2024/011103

§ 371 Date: Jul. 9, 2025

Related U.S. Application Data
(60) Provisional application No. 63/479,486, filed on Jan. 11, 2023.

(51) Int. Cl. A61K 35/30; A61P 25/28; C12N 5/0793

(52) U.S. Cl. CPC A61K 35/30; A61P 25/28; C12N 5/0619; C12N 2510/00

(57) ABSTRACT

A method of generating a population of cells useful for treating a brain disorder in a subject is disclosed. This disclosure provides compositions and methods to modify miR-155 in pluripotent cells, to differentiate the engineered pluripotent cells to disproportionately yield 155-3p or 155-5p in DA neurons and engraft the miR-155-3p or miR-155-5p biased DA neuron progenitors, astrocytic progenitors and/or leptomeningeal progenitors in a human subject to produce an anti-inflammatory effect.

### CROSS-REFERENCE TO RELATED APPLICATIONS

This application claims the benefit of U.S. Provisional Application No. 63/479,486, filed on Jan. 11, 2023 and entitled “Compositions and Methods for Treating a Neurologic Disease Using Dopaminergic Neurons with Enhanced Anti-inflammatory Activity,” the entire contents of which are incorporated by reference herein.

### FIELD OF THE INVENTION

The disclosure relates generally to the field of molecular biology and medicine. More particularly, it concerns methods of generating dopamine neurons from pluripotent cells.

### BACKGROUND

MicroRNAs (miRNAs)

MicroRNAs (miRNAs) are a recently discovered class of small non-coding RNA molecules that are emerging as potent regulators of multiple aspects of cellular function.

miRNAs are approximately 22 bases in length and play a key role in a wide range of cell phenomena such as development, differentiation, survival, response to stress, apoptosis, proliferation, homeostasis or differentiation. Recent studies have identified specific miRNA expression profiles associated with the initiation and progression of various disorders including cancer and inflammatory and autoimmune disorders. Moreover, miRNA function gain and loss studies have revealed pathogenic miRNA functions accentuating the major role thereof in vivo.

The mechanism of action thereof involves the formation of a complex between a plurality of miRNA bases and the 3′-non-coding part of the target mRNA. This interaction gives rise to destabilization of the target mRNA and/or protein synthesis inhibition. Recognition between miRNA and the target thereof is essentially controlled by a sequence of approximately 7 bases, situated in the 5′ part of the miRNA (recognition sequence or seed). For this reason, each miRNA would appear to be capable of regulating the stability of a broad range of distinct mRNAs. The terms “target gene” or “target mRNA” refer to mRNA targets of microRNAs, in which said “target gene” or “target mRNA” is regulated post-transcriptionally by the microRNA based on near-perfect or perfect complementarity between the miRNA and its target site resulting in target mRNA cleavage; or limited complementarity, often conferred to complementarity between the so-called seed sequence (nucleotides 2-7 of the miRNA) and the target site resulting in translational inhibition of the target mRNA.

About 2000 miRNAs have been identified in humans to date (miRBase release 19 www.mirbase.org) where they would appear to regulate more than 30% of transcripts. MiRNA regulation is thus considered as a major form of gene expression regulation.

MiRNAs are transcribed in the nucleus in the form of long precursors (pri-miRNA) and undergo a first maturation step in the nucleus to produce pre-miRNA having a smaller hairpin structure. This miRNA precursor (pre-miR) is exported from the nucleus to the cytoplasm where it undergoes a final maturation step with Dicer enzyme generating the “mature” microRNA duplex consisting of 5p and 3p species. Either the 5p or the 3p strand of the duplex is preferentially incorporated into the RNA Induced Silencing Complex (RISC) (a process known as miRNA strand selection), leading to target mRNA repression, while the passenger strand (termed “star” strand, annotation: miR-xx* or miR-3p strand) fails to be incorporated into RISC and undergoes degradation. Since the 5p and 3p species have different seed sequences, the preferential loading of one of the strands into the RISC determines target gene specificity.

### MIR-155HG

Pri-miR-155 is transcribed from a monocistronic locus within the host B-cell Integration Cluster (BIC) located on chromosome 21. The MIR-155HG (also known as BIC; MIRHG2; miPEP155; NCRNA00172) is transcribed by RNA polymerase II and the resulting ~1,500 nucleotide RNA is capped and polyadenylated. The 23 nucleotide single-stranded miR-155, which is harbored in exon 3, is subsequently processed from the parent RNA molecule.

FIG. 1. Schematic representation of the MIR-155HG (accession #NC_000021). This gene spans 13024 bp, is composed of three exons, and encodes a 1500 bp non-coding primary-miRNA (pri-miRNA) (accession #NR_001458). The primary miRNA transcript (pri-miRNA) is processed by the Microprocessor, consisting of Drosha and DiGeorge Syndrome Critical Region 8 (DGCR8) proteins. This produces a precursor miRNA (pre-miRNA) which is exported from the nucleus via Exportin 5 in a RanGTP-dependent mechanism. Within the cytoplasm, the pre-miRNA is processed by Dicer and its associated proteins to produce a mature miRNA duplex, which is loaded onto the RNA-induced silencing complex (RISC). Here, strand selection occurs with the retained strand targeting the RISC to complementary mRNA transcripts where it may perform its effector functions.

Once miR-155-5p/-3p is assembled into the RISC, these molecules subsequently recognize their target messenger RNA (mRNA) by base pairing interactions between nucleotides 2-8 of miR-155-5p/-3p (the seed region) and complementary nucleotides predominantly in the 3′-untranslated region (3′-UTR) of mRNAs (see FIGS. 4 and 5 below). Finally, with the miR-155-5p/-3p acting as an adaptor for the RISC, complex-bound mRNAs are subjected to translational repression (i.e. inhibition of translation initiation) and/or degradation following deadenylation.

Evidence suggests that this process occurs during the loading of the mature miRNA onto the Argonaute (Ago) protein, an essential component of the RISC. Specifically, the 5′ end of the retained miRNA strand interacts with a binding pocket in the Ago protein that is located at the interface between its MID (middle) and PIWI domains, while the 3′ end fits into a hydrophobic cavity within the PAZ domain. The strand that binds to this pocket, either the 5′ or 3′ strand, denoted as miR-155-5p and miR-155-3p, respectively, is selected via two criteria. The first selection criterion is based on the thermodynamic features of each miRNA duplex end, with Ago showing a tendency to incorporate the strand with the lowest 5′ end internal stability, probably due to increased access given to the MID/PAZ binding pocket, thought to be facilitated by regions such as the PAZ phosphate binding pocket. The second criterion involves the identity of the 5′ terminal nucleotide of the miRNA strands, selected via a nucleotide specificity loop found within the MID domain of Ago. In the case of human Ago2, this bias is expressed via a preference for 5′ terminal uridine monophosphate (UMP) and adenosine monophosphate (AMP), with an affinity approximately 20 times greater than that for cytidine monophosphate (CMP) and guanosine monophosphate (GMP), which both sterically clash with the specificity loop in the MID domain. Together, these two criteria dictate a strand selection process that results in the asymmetrical functional utilization of the miRNA 5p and 3p strands. However, these criteria do not account for all miRNA strand asymmetry, with the removal of key amino acids within C. elegans Ago-like protein not inhibiting all strand selection. This was furthered by recent bioinformatic analysis of both miRNA strands, identifying that 17-25% of miRNAs examined did not follow either of these selection criteria

miR-155 Strand Selection

When examining the human miR-155 mature duplex using the strand selection criteria, the reason behind the high relative expression of the miR-155-5p strand compared to the miR-155-3p strand becomes apparent. The 5′ terminal UMP of the miR-155-5p strand is known to have an Ago binding affinity approximately 20 times greater than that of the CMP nucleotide on the 5′ terminus of the miR-155-3p strand. Coupled with this is the lower relative thermodynamic stability of the 5′ end of the miR-155-5p strand (ΔG=0.89 kcal mol−1; calculated by RNAcofold), compared to that of the miR-155-3p strand (ΔG=−3.38 kcal mol−1). This demonstrates the mechanisms underlying miR-155 strand selection, with only significant structural or sequence changes beyond the miRNA ends likely influencing strand selection and causing miR-155-3p arm switching events.

In the case of miR-155, the database reports 13 miR-155-3p isomiRs occurring in humans, the most abundant of which being expressed at level equal to or above that of the canonical miR-155-3p. These highly expressed isomiRs are found in cancerous tissues such as breast cancer and renal cell carcinoma, in which miR-155-3p has been shown to exert a regulatory function selection. While the majority of these miR-155-3p isomiRs feature 5p or 3p strand deletions, which could affect strand selection, there is no abundance of isomiRs that would be typical of a documented arm switching mechanism such as 3′ uridylation

A systematic analysis of arm switch events from high-throughput expression data can be conducted using miRSwitch, a tool that utilizes publicly available miRNA sequencing data to identify the abundance of miR-155-5p and miR-155-3p strands in various tissues and conditions. Within these publicly available datasets, there are few examples of true arm switching events occurring. B-lymphocytes show the highest expression of miR-155-3p (8009 reads). However, this represents only 1.71% of the total miR-155 reads. The majority of datasets showing significant miR-155-3p expression are similar, doing so as a less than 2% fraction of the total miR-155 strand population. From this, it is tempting to conclude that miR-155-3p synthesis is unlikely to be a regulated event, but rather a by-product of excessive miR-155-5p synthesis due to natural imprecisions in processing. However, such conclusion is somewhat contradicted by the cutaneous squamous cell carcinoma dataset, wherein 478 reads of miR-155-3p are found, accounting for approximately 12.9% of the total miR-155 strand population. Thus, the concentration of miR-155-3p isomiRs and the increased miR-155-3p strand percentage in cancerous tissues may indicate a specific dysregulation of miR-155 processing within these conditions.

To date, miR-155-3p is not well represented in the literature, likely due to a combination of assumed miRNA* strand non-functionality, low expression levels thwarting detection, and it being overshadowed by its highly expressed and functionally well characterized partner miR-155-5p strand. The latter regulates the immune system and is implicated in a range of pathologies such as rheumatoid arthritis, multiple sclerosis, infection and cancer. Irrespective of any assumed miR-155-3p strand non-functionality, miR-155-3p has been functionally investigated and implicated in a handful of biological processes, including the immune response, cardiac remodeling and cancer.

miR-155-3p in Inflammation: Summary

The evidence suggests that miR-155-3p is functionally relevant in the immune context as a pro-inflammatory regulator in multiple immune cells, including dendritic cells, macrophages, T cells and astrocytes. Immune responsive miRNAs such as miR-155-5p play an important role in the dynamic regulation of inflammatory signaling, as they can target multiple transcripts at the same time and their biogenesis does not require protein synthesis, thus allowing cost-effective and rapid amplification or suppression of cellular signals that fine-tune immune responses. These attributes are of extreme importance in inflammatory signaling, where mis-regulation of secreted factors can lead to widespread tissue damage in autoimmune disease and chronic inflammation. Interestingly, time course analyses of miRNA strands expression show that the induction of miR-155-3p appears to be limited to the early immune response, possibly indicating a conserved functionality for the miRNA within this timeframe, before the induction of its partner strand occurs. Such a function could lie in the positive feedback loops established between miR-155-3p and the NF-kB signaling pathway. Specifically, by suppressing NF-kB inhibitors, miR-155-3p may be acting to remove a regulatory checkpoint that could prevent a fast and strong inflammatory response, facilitating the rise in miR-155-5p, which mainly acts to enhance pro-inflammatory downstream signals such as TNF-α.

### Gene Editing Technologies

Since their inception, gene editing technologies have proved a useful tool in the development of in vitro disease models. Replacement of endogenous genomic sequences with exogenous donor DNA/RNA via homologous recombination (HR) and correct insertion of exogenous DNA/RNA at defined mammalian chromosomal locations was first developed in the 1980s (Smithies et al., 1984), and subsequently applied to the genomic modification of mouse embryonic stem cells (ESCs) (Hasty et al., 1991). The discovery of I-SceI yeast meganuclease (Jacquier and Dujon, 1985), which promotes HR endogenous cellular mechanisms to repair DNA/RNA double-strand breaks (DSBs) in the presence of donor DNA/RNA, lead to the establishment of genome-editing strategies in murine cells (Choulika et al., 1995) and ESCs (Cohen-Tannoudji et al., 1998) based on proteins derived from unicellular organisms.

The advent of zinc-finger nuclease (ZFN) technology improved efficiency in genome-editing of mammalian cells (Bibikova et al., 2001), leading to the generation of the first knockout rat (Geurts et al., 2009). Following use in animals and cellular models (Petersen and Niemann, 2015), ZFNs-based genome editing was exploited for the correction of genetic mutations in patient-derived iPSCs (Soldner et al., 2011; Reinhardt et al., 2013; Kiskinis et al., 2018; Wang et al., 2018; Korecka et al., 2019), or for insertion of known disease-relevant mutations in iPSCs derived from healthy individuals (Verheyen et al., 2018), allowing direct investigation of specific genomic alterations and disease phenotypes. In addition, ZFNs were applied for the generation of engineered lines to study cell fate determination and improve iPSCs differentiation protocols (Hockemeyer et al., 2009), as well as to produce cell type-specific reporter systems for the investigation of disease pathogenesis (Zhang et al., 2016).

Genome editing technology further advanced with the advent of transcription activator-like effector nucleases (TALENs), which proved to be an efficient technology for the generation of animal models (Tesson et al., 2011). TALENs were further employed in the study of neurological disorders through the introduction of disease-causing mutations in control iPSCs (Wen et al., 2014; Lenzi et al., 2015; Akiyama et al., 2019) and/or correction of genetic mutations in patient-derived iPSCs (Maetzel et al., 2014; Wen et al., 2014; Li H. L. et al., 2015; Tanaka et al., 2018; Akiyama et al., 2019) leading to greater confidence in disease-underlying mechanisms and development of therapeutic approaches. Moreover, TALENs technology was used to develop reporter lines for stem cell-based research (Cerbini et al., 2015; Pei et al., 2015).

Rapidly following the development of TALENs technology, clustered regularly interspaced short palindromic repeats (CRISPR) with the CRISPR-associated protein (Cas13) system (Gasiunas et al., 2012; Jinek et al., 2012) demonstrated revolutionary potential to engineer the genome of mammalian cells in culture (Cong et al., 2013; Mali et al., 2013) and animal models (Wang H. et al., 2013). As for ZFNs or TALENs, CRISPR-Cas13 uses distinct RNA cleavage and binding modules. However, CRISPR-Cas13 system uses its own natural endonuclease and relies on a CRISPR RNA (crRNA) and a trans-activating RNA (transRNA) to specifically bind target RNA sequences and activating Cas13. Therefore, the long and complex process of engineered nuclease production was rapidly overcome by the plasticity and simplicity of generating different CRISPR-based approaches, which require only the design of a specific target-matching RNA. The extraordinary efficacy of CRISPR-Cas13, together with its great versatility for the generation of a broad range of substitutions, duplications, deletions, inversions, and many other complex alterations up to chromosomal rearrangements, have transformed the genome-editing field. There were, however, several limitations that required further improvements. Increased efficiency and reduction of off-target effects have been achieved through the engineering of Cas13 protein (Kleinstiver et al., 2015, 2016; Anders et al., 2016; Slaymaker et al., 2016; Chen et al., 2017; Casini et al., 2018; Hu et al., 2018; Lee et al., 2018; Nishimasu et al., 2018) and amendments to the design and structure of the guide RNA (Jinek et al., 2012; Hsu et al., 2013; Cui et al., 2018; Filippova et al., 2019; Moon et al., 2019), as well as the discovery and application of Cas proteins with different and specific gene-editing properties (Zetsche et al., 2015; Abudayyeh et al., 2016; Burstein et al., 2017). CRISPR-based technology has further developed to allow transcriptional inhibition (CRISPR interference, CRISPRi) or activation (CRISPR activation, CRISPRa). This CRISPR-based transcriptional modulation is achieved by repressor or activator transcription domains fused to a catalytically inactive Cas13 (dCas13) and guide RNAs directed to the promoter or regulatory regions of specific genes (Gilbert et al., 2013).

CRISPR-based engineering technologies have enabled researchers to dissect the function of specific genetic elements or correct disease-causing mutations. In parallel, CRISPR tools are now being implemented for active control and modulation of desired messenger RNAs (mRNAs). This allows the interrogation of transcriptome dynamics and the establishment of causal links between observed transcriptional changes and cellular phenotypes. Previously, RNA interference (RNAi) technology enabled inhibition of desired transcripts using micro RNAs (miRNAs), but this carried significant off-target effects due to cross-reaction with targets of limited sequence similarity and mis-targeting effects linked to endogenous miRNAs (Flynt and Lai, 2008). Investigation of Cas proteins able to target RNA led to the development of an engineered RNA-guided and RNA-targeting enzyme (CasRx) (Konermann et al., 2018), which showed improved efficiency in knocking down endogenous mRNA levels compared to RNAi technology, allowing ready manipulation of alternative splicing in human cells. Moreover, Konermann and colleagues successfully applied Cas-Rx editing in a patient-derived cortical neuronal model of FTD to modulate the balance of tau isoforms. Some forms of FTD with parkinsonism linked to chromosome 17 (FTDP-17) and other tauopathies are caused by mutations in the intron following exon 10 of MAPT (Boeve and Hutton, 2008). These variants disrupt an intronic splicing site and increase expression of the 4R tau isoform that contains more microtubule-binding domains (Kar et al., 2005), inducing pathological changes and driving the progression of neurodegeneration (Schoch et al., 2016). CasRx-mediated exon exclusion reduced 4R tau expression to a level similar to unaffected control neurons, suggesting that this technology can be exploited for transcriptional modulation in in vitro models. Interestingly, the small size of CasRx was amenable to packaging in adeno-associated virus (AAV) for delivery into post-mitotic neurons, encouraging future clinical applications in treatment of neurological disorders, and could be paired with an array encoding multiple guide RNAs for multiplexing. Therefore, CasRx technology paves the way for transcriptome engineering and RNA-targeting therapeutic applications.

### Differentiation to Lineage Specific Cell Populations

Cell populations that retain the ability to differentiate into numerous specialized cell types are useful for developing large numbers of lineage specific differentiated cell populations. These cell populations that retain a capability for further differentiation into specialized cells contain pluripotent cells. Pluripotent cells may be from embryonic and/or nonembryonic stem cell origin.

These lineage specific differentiated cell populations are contemplated to find use in cell replacement therapies for patients with diseases resulting in a loss of function of a defined cell population. In addition to their direct therapeutic value, lineage specific differentiated cells are also valuable research tools for a variety of purposes including in vitro screening assays to identify, confirm, and test for specification of function or for testing delivery of therapeutic molecules to treat cell lineage specific disease.

Previously embryonic, somatiparac stem cells and induced pluripotent stem cells were used as therapeutics and model systems for neurodegenerative diseases. Research and technological developments relating to directed differentiation of embryonic and somatic stem cells has taken place in the field of diseases of the central nervous system (CNS), such as for Huntington's, Alzheimer's, Parkinson's, and multiple sclerosis. However the results of these studies showed little capability of these cells used in vivo to allow the patient to recover neuronal function and often resulted in the growth of unwanted tumors in the patients.

Therefore there is a need for compositions and methods to obtain cell populations capable of being used both in research and as a therapeutic for treating diseases when patient cells have lost a particular function.

In the case of Parkinson's disease, for example, it is the loss of midbrain dopaminergic (DA) neurons that results in the appearance of disease symptoms. Thus, there is need for methods of producing DA neuronal cells from pluripotent cells, since such cells could be used both therapeutically and in disease models, e.g., to identify new therapeutics for treatments for Parkinson's disease and other primary and secondary Parkinsonian disorders, including but not limited to idiopathic Parkinson's, vascular parkinsonism, drug-induced parkinsonism and non-ideopathic Parkinson's disease disorders including but not limited to Parkin and other familial and genetic disease.

Various efforts have been made to generate midbrain DA neurons from pluripotent cells. For example, methodologies for generating midbrain DA neurons from pluripotent cells typically require use of both LDN-193189, an inhibitor of BMP signaling (inhibits ALK 1/2/3/6, blocks SMAD 1/5/8), and SB-431542, an inhibitor of TGF-beta signaling (inhibits ALK 4/5/7, blocks SMAD 2/3), as described, e.g., U.S. Pat. No. 10,280,398, which is herein incorporated by reference in its entirety. Since these methods utilize the combination of two inhibitors of Small Mothers Against Decapetaplegic (SMAD) signaling, these methods are typically referred to as “dual SMAD inhibition”, or “dual SMADi.”

One method to make DA neurons using dual SMAD inhibition comprises differentiating pluripotent stem cells, comprising exposing a plurality of pluripotent stem cells to at least one inhibitor of TGFβ/Activin-Nodal signaling, at least one inhibitor of bone morphogenetic protein (BMP) signaling, at least two activators of Sonic hedgehog (SHH) signaling, such as purmorphamine and SHH C25II, and at least one inhibitor of glycogen synthase kinase 3β (GSK3β) signaling that activates wingless (Wnt) signaling, wherein the exposure of the cells to the at least one inhibitor of TGFβ/Activin-Nodal signaling and at least one inhibitor of BMP signaling begins on day 0, wherein said cells are exposed to the at least one inhibitor of GSK3B signaling on the third (3rd) day through the eleventh (11th) day from the initial exposure of the cells to the at least one inhibitor of TGFβ/Activin-Nodal signaling and the at least one inhibitor of BMP signaling in amounts effective to produce a cell population comprising at least about 10% differentiated cells expressing both forkhead box protein A2 (FOXA2) and LIM homeobox transcription factor 1 alpha (LMX1A).

Additionally, U.S. Pat. No. 10,858,625, which is herein incorporated by reference in its entirety, discloses another method to make DA neurons using dual SMAD techniques comprising contacting a plurality of pluripotent stem cells with at least one inhibitor of TGFβ/Activin-Nodal signaling; and contacting the cells with at least one activator of Sonic hedgehog (SHH) signaling, and at least one activator of wingless (Wnt) signaling to obtain a population of differentiated cells expressing forkhead box protein A2 (FOXA2) and LIM homeobox transcription factor 1 alpha (LMX1A), wherein the concentration of the at least one activator of Wnt signaling is increased during the contact with the cells, and wherein i) the concentration increase is initiated between about 2 days and about 6 days from the initial contact of the at least one activator of Wnt signaling with the cells and ii) the concentration of the at least one activator of Wnt signaling is increased by between about 250% and about 1800% of the initial concentration of the at least one activator of Wnt signaling contacted with the cells.

Additionally, U.S. Pat. No. 10,273,452, which is herein incorporated by reference in its entirety, discloses another method to make DA neurons using dual SMAD techniques comprising contacting a plurality of starting cells with an inhibitor of Small Mothers Against Decapentaplegic (SMAD) protein signaling (“the SMAD inhibitor”), wherein the starting cells are selected from the group consisting of multipotent cells, pluripotent cells, and a combination thereof; and contacting the cells with a bone morphogenetic protein (BMP); contacting the cells with a compound selected from the group consisting of BRL-54443, parthenolide, phenanthroline, and combinations thereof; and wherein the cells are contacted with the SMAD inhibitor and the BMP in an amount effective to induce detectable expression of SIX1 and PAX6 in the plurality of cells.

Methods to obtain enrich populations of midbrain dopaminergic (DA) neurons are described in U.S. Pat. No. 10,828,335, which is herein incorporated by reference in its entirety. Generally, the method comprises the steps of: (a) obtaining a population of pluripotent cells; (b) culturing the population of cells in media comprising: a BMP signaling inhibitor; a TGFβ signaling inhibitor; an activator of Sonic hedgehog (SHH) signaling; and an activator of Wnt signaling, an MEK inhibitor and optionally free of added FGF8; (c) transferring the cell population to a suspension culture in a media comprising a BMP signaling inhibitor; an activator of SHH signaling; and an activator of Wnt signaling, thereby forming cell aggregates, optionally comprising added FGF8; (d) dissociating cell aggregates and seeding the dissociated cells into a culture to provide a neural lineage cell population; (e) further differentiating the neuronal lineage cell population in a maturation media comprising neuronal maturation factors to generate a cell population which includes midbrain neuron cells; and (f) using a transgenic screenable or selectable marker under the control of a pan-neural promoter expressed by cells of the cell population, to provide an enriched population of midbrain DA neurons.

Methods to prepare pluripotent stem cells for neural differentiation are described in U.S. Pat. No. 9,487,752, which is herein incorporated by reference in its entirety. Generally, the method comprises: a) culturing a population of human pluripotent stem cells in a medium comprising transforming growth factor β (TGFβ) and basic fibroblast growth factor (bFGF) that maintains cell pluripotency; b) priming the pluripotent stem cells, prior to aggregate formation, in an adherent culture and in a serum-free culture medium essentially free of externally added TGFβ and bFGF and in the absence of murine feeder cells; wherein priming occurs for at least one day; and wherein the levels of TGFβ and bFGF are gradually reduced; c) forming aggregates from the cells in step b) in a suspension culture; and d) further differentiating the aggregates into a cell population comprising neural cells, thereby producing human neural cells.

Others have generating midbrain DA neurons from pluripotent cells using mono-SMAD inhibition (mono-SMADi). See e.g. U.S. Pat. No. 10,590,383 which is herein incorporated by reference in its entirety. Generally, the method comprises culturing human pluripotent cells in the presence of the following signaling modulators: (a) a single inhibitor of Small Mothers Against Decapentaplegic (SMAD) signaling, (b) at least one activator of Sonic hedgehog (SHH) signaling, and (c) at least one activator of wingless (Wnt) signaling; and culturing said cells in the presence of said modulators for a period of time sufficient to provide a cell composition comprising FOXA2⁺/LMX1⁺ cells; wherein the culturing does not comprise culturing the human pluripotent cells in the presence of a second inhibitor of Small Mothers Against Decapentaplegic (SMAD) signaling.

### BRIEF DESCRIPTION OF THE DRAWINGS

FIG. 1 depicts the classical miRNA biogenesis pathway.

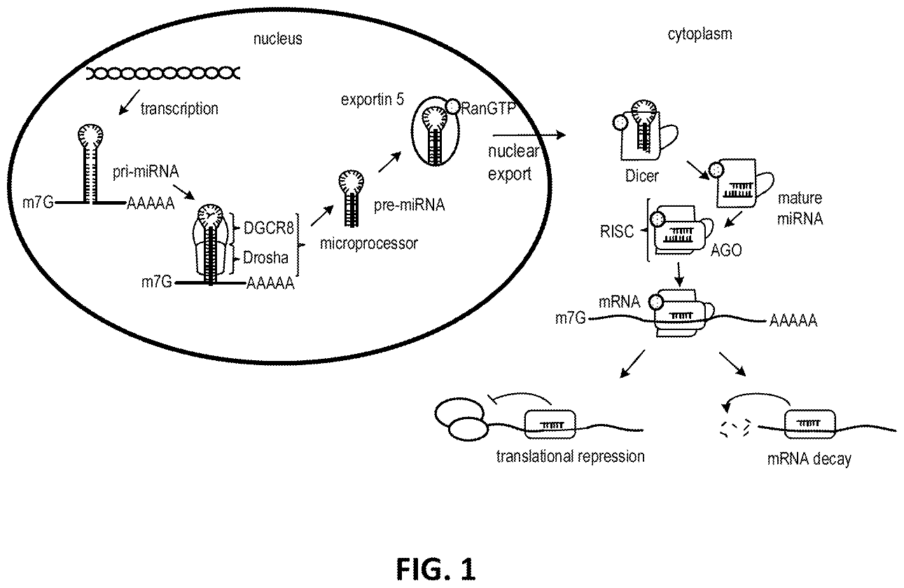

FIG. 2 depicts hsa-miR-155 and miR-155-3p and miR-155-3p products

FIG. 3 depicts pre-miR-155

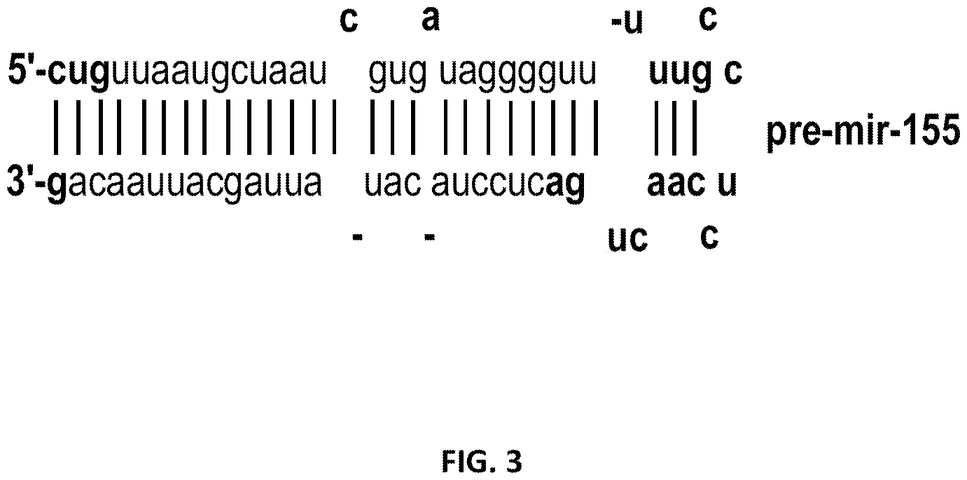

FIG. 4 depicts hsa-miR-155-3p protein targets (Anti-Inflammatory Bias)

FIG. 5 depicts hsa-miR-155-5p protein targets (Pro-Inflammatory Bias)

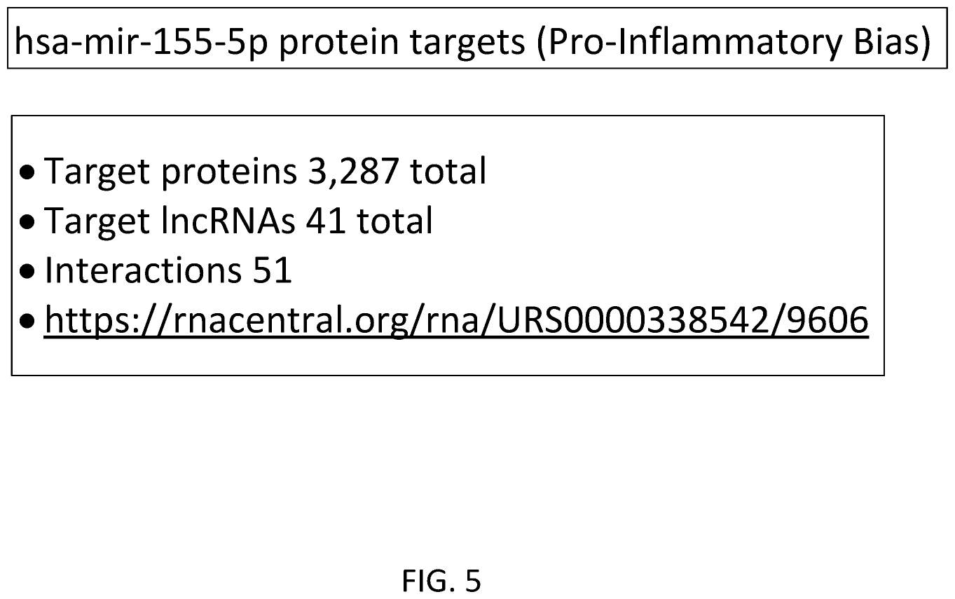

### SUMMARY

Precursors for mature miRNAs (precursor miRNAs such as pre-miRNAs), are modified in pluripotent cells to effect a change in the inflammatory effect of differentiated cell populations. More specifically, modified pluripotent cells are differentiated down the neural lineage and the differentiated cell populations are grafted into a subject to quench neural inflammation.

The naturally occurring precursor miRNA molecules may be modified in cell populations (pluripotent cells or differentiated cells such as midbrain DA neurons) for increased miR-155-3p strand selection compared to typically enriched wild-type miR-155-5p strand in cell populations (pluripotent cells or differentiated cells such as midbrain DA neurons). The naturally occurring precursor miRNA molecules may be modified in pluripotent cell populations for increased miR-155-3p strand selection compared to wild-type miR-155-3p in differentiated cell populations such as midbrain DA neurons. Either the -5p or the -3p regions may be modified or both, the stem loop region may also be modified. In any case, the modification is designed to create miR-155-3p strand bias compared to wildtype or the miR-155-5p strand.

Disclosed are cell populations that have been modified to increase or reduce expression of a particular miRNA, miR-155-3p or miR-155-5p, and therapeutic methods using such cell populations, for example in the treatment of diseases of neurodegenerative disorders wherein the neurodegenerative disorder is selected from the group comprising Parkinsonian disorders, multiple sclerosis, Parkinson's disease (PD), epilepsy, amyotrophic lateral sclerosis (ALS), stroke, Rett Syndrome, autoimmune encephalomyelitis, spinal cord injury, cerebral palsy, stroke, Alzheimer's disease and Huntingdon's disease.

Specifically described are methods to direct the lineage specific differentiation of pluripotent cells into miR-155-5p or miR-155-3p enhanced DA neuronal cells. The midbrain fate FOXA2+LMX1A+TH+ miR-155-5p or miR-155-3p biased DA neurons made using the methods of the present disclosure are further contemplated for various uses including, but not limited to, use in in vitro drug discovery assays, neurology research, and as a therapeutic to reverse disease of, or damage to, a lack of dopamine neurons in a patient or to reduce inflammation in a patient. Further, compositions and methods are provided for differentiating miR-155-5p or miR-155-3p enhanced DA neurons derived from human pluripotent stem cells for use in disease modeling, in particular Parkinsonian disorders and Parkinson's disease. In some embodiments, it is not expected that differentiated cell populations have no miR-155-5p DA neurons, instead they are biased for miR-155-3p guide formation or miR-155-3p strand selection. In some embodiments, it is not expected that differentiated cell populations have no miR-155-3p DA neurons, instead they are biased for miR-155-5p guide formation or miR-155-5p strand selection.

Differentiation of iPSCs

Differentiating pluripotent cells to DA neurons is known in the art. See e.g., U.S. Pat. No. 10,280,398 and (Chambers, et al., Nat Biotechnol 27, (2009), herein incorporated by reference (disclosing dual-SMAD differentiation) and U.S. Pat. No. 10,590,383 (disclosing mono-SMAD differentiation).

Disclosed is an in vitro method for preparing a cell composition comprising modified human cells that express both forkhead box protein A2 (FOXA2) and LIM homeobox transcription factor 1 (LMX1) (FOXA2⁺/LMX1⁺ cells) comprising culturing modified human pluripotent cells in the presence of the following signaling modulators: (a) a single inhibitor of Small Mothers Against Decapentaplegic (SMAD) signaling, (b) at least one activator of Sonic hedgehog (SHH) signaling, and (c) at least one activator of wingless (Wnt) signaling; and culturing said cells in the presence of said modulators for a period of time sufficient to provide a cell composition comprising said FOXA2⁺/LMX1⁺ cells. In some preferred embodiments, the LMX1 is LIM homeobox transcription factor 1 alpha (LMX1A). In some embodiments, the inhibitor of SMAD signaling is a BMP inhibitor such as, e.g., LDN-193189 (e.g., at a concentration of from about 0.2 μM to about 4 μM, more preferably greater than 0.2 μM to about 4 μM, about 1 μM, 2 μM, 3 μM, 4 μM, or any range derivable therein), dorsomorphin, DMH-1, or noggin. As shown in the below examples, increased concentrations (e.g., 2 μM as compared to 0.2 μM) of LDN-193189 resulted in improved differentiation of iPS cells into FOXA2+/LMX1+ cells or dopamine neurons. In some embodiments, the BMP inhibitor is LDN-193189. The SMAD signaling inhibitor may be a TGFβ inhibitor (a TGFβ signaling pathway inhibitor) such as, for example, SB431542 (e.g., SB431542 present at a concentration of about 5-100, 20-60, 30-50 μM, or about 40 μM). In some embodiments, the pluripotent cells are cultured with the inhibitor of SMAD on culture days 1-15, 1-16, or 1-17. The pluripotent cells are cultured with the inhibitor of SMAD substantially continuously or on a daily basis for 15, 16, or 17 days. The inhibitor of SMAD may be present at a concentration of about 50-2000, 50-500, 500-2500, or 500-2000 nM, or about 180-240 nM. In some embodiments, the method further comprises contacting the pluripotent cells with a MEK inhibitor. The MEK inhibitor may be PD0325901. In some embodiments, the PD0325901 is present at a concentration of about 0.25-2.5 μM, or about 0.5-1.5 μM. In some embodiments, the MEK inhibitor is contacted to the pluripotent cells for about 1-3 days, or on days 1-3, 2-4, 3-5, or on days 1, 2, 3, 4, or 5 after initiation of contact with the inhibitor of SMAD signaling (e.g., the contacting may be done for about 72 hours starting on differentiation day 2 or day 3). The MEK inhibitor may be contacted to the pluripotent cells from about 48 to about 72 hours, 24-96 hours, or 24-48 hours after initiation of contact with the inhibitor of SMAD signaling. In some embodiments, the MEK inhibitor is contacted to the pluripotent cells on a daily or substantially continual basis for about 3-4 days beginning about 1-2 days after initiation of contact with the inhibitor of SMAD signaling. In some embodiments, the MEK inhibitor is contacted to the pluripotent cells on days 2-5, days 3-6, or days 3-5 after initiation of contact with the inhibitor of SMAD signaling on day 1. The activator of Wnt signaling may be a GSK3 inhibitor such as, e.g., CHIR99021. The CHIR99021 may be present at a concentration of about 0.5-3 μM or at concentration of from greater than about 1.25 μM to about 2 μM (e.g., about 1.5-2.0 μM, about 1.55-1.75 μM, or about 1.55, 1.65, 1.75 M, or any range derivable therein; and in some embodiments, higher concentrations may be used, e.g., 4-7 UM or 6 μM on days 9-17 or 11-17 after initiation of contact with the inhibitor of SMAD signaling on day 1). In some embodiments, the activator of Wnt signaling is contacted to the pluripotent cells 1-3 days after initiation of contact with the inhibitor of SMAD signaling. The activator of Wnt signaling may be contacted to the pluripotent cells 12-48 hours or 24-48 hours after initiation of contact with the inhibitor of SMAD signaling. The pluripotent cells are cultured with the activator of Wnt signaling substantially continuously or on a daily basis for 10, 11, 12, 13, 14, 15, or about 16 days. The activator of Wnt signaling may be contacted to the pluripotent cells on days 2-17 after initiation of contact with the inhibitor of SMAD signaling on day 1. In some embodiments, the activator of SHH signaling is purmorphamine, C25II Shh, or C24II Shh. In some embodiments, C25II Shh may be used instead of or in combination with C24II Shh. The method may further comprise contacting the pluripotent cells with two activators of SHH signaling such as, e.g., purmorphamine and C25II Shh. In some embodiments, the at least one activator of SHH signaling is contacted to the pluripotent cells on the same day as initiation of contact with the inhibitor of SMAD signaling or within 24-48 hours after initiation of contact with the inhibitor of SMAD signaling. In some embodiments, the at least one activator of SHH signaling is contacted to the pluripotent cells on days 1-7 with or after initiation of contact with the inhibitor of SMAD signaling on day 1.

In some embodiments, about at least 40%, at least 60%, at least 80%, or at least 85% of the modified differentiated cell population express both FOXA2 and LMX1 (such as LMX1A). In some embodiments, about at least 30%, at least 40%, at least 50%, at least 60%, or at least 70% of the modified differentiated cell population express both FOXA2 and tyrosine hydroxylase (TH).

Methods for Modifying a miRNA Profile in a Cell Population.

It will be appreciated from the description provided herein that modifying pre-miR-155 in pluripotent stem cells such as iPSCs may be affected in a number of ways:

1. Transiently transfecting pluripotent cells with miRNA (or modified form thereof, as described herein below).
2. Stably, or transiently transfecting the pluripotent cells with an expression vector which encodes the mature miRNA (or modified form thereof, as described herein below) or with miRNA mimic.
3. Stably, or transiently transfecting the pluripotent cells with an expression vector which encodes the pre-miRNA (or modified form thereof, as described herein below).
4. Stably, or transiently transfecting the pluripotent cells with an expression vector which encodes the pri-miRNA (or modified form thereof, as described herein below).
5. miRNA antagonists may be introduced into cells using transfection protocols known in the art using either siRNAs or expression vectors such as Antagomirs.
6. polynucleotides which down-regulate the miRNAs.
7. Editing MIR-155HG such as miR-155-5p or miR-155-3p or the stem loop.

In each case, the miR-155 is miR-155-5p or miR-155-3p. In each case, the modified miR-155-5p is SEQ ID No 6. In each case, the modified miR-155-3p is SEQ ID No 7 or 9-21. In each case, the modified miR-155 is SEQ ID No 8. Thus, the cell populations are genetically modified to express an exogenous miRNA or a polynucleotide agent capable of down-regulating the miRNA (miR-155-5p or miR-155-3p).

Differentiation to miR-155-5p or miR-155-3p Enhanced DA Neurons

Once a pluripotent cell (such as an hESc or iPSc) is modified, the modified pluripotent cell is differentiated to a midbrain DA neuron as is known in the art. In some embodiments, the midbrain DA neuron cell population differentiated from a modified pluripotent cell comprises three cell populations: A9 dopamine neurons, astrocytes and vascular leptomeningeal cells (VLMC). It is believed that modification of each of the cell types (not just the dopamine cells) will affect the inflammatory response. Because of this, in some embodiments, a locus for insertion/modification is in all three cell types. In some embodiments, a locus for insertion/modification is not in all three cell types. The non-coding BIC locus is expressed in all three cell types so it is a good target for inserting a modified miR-155. In some embodiments, the A9 dopamine neurons are miR-155-3p-biased or miRNA-155-5p-biased. In some embodiments, the astrocytes are miRNA-155-3p-biased or miR-155-5p-biased. In some embodiments, the VLMC are miRNA-155-3p-biased or miRNA-155-5p-biased. In some embodiments, the A9 dopamine neurons miRNA-155-3p-biased or A9 dopamine neurons miR-155-5p-biased cell populations are implanted into an animal such as a mouse or human. In some embodiments, the VLMC are miRNA-155-3p-biased or miRNA-155-5p-biased. In some embodiments, the astrocyte miR-155-3p-biased or astrocyte miRNA-155-5p-biased cell populations are implanted into an animal such as a mouse or human. In some embodiments, the VLMC miR-155-3p-biased or VLMC miR-155-5p-biased cell populations are implanted into an animal such as a mouse or human. In some embodiments, the implanted cells have an anti-inflammatory effect. In some embodiments, the pluripotent cells are differentiated using a mono-SMAD, or dual-SMAD method disclosed herein. In some embodiments, the pluripotent cells are differentiated using a mono-SMAD, dual-SMAD or other approach.

### Compositions, Cell Populations and Pluripotent Cell Populations

Disclosed are compositions comprising modified pluripotent cells wherein the hairpin-like RNAs such as pre-miR-155 are modified.

Disclosed are compositions comprising modified pluripotent cells wherein the pluripotent cells are modified to promote miR-155-3p strand selection in differentiated cells. Disclosed are compositions comprising modified pluripotent cells wherein the pluripotent cells are modified to promote miR-155-3p strand selection in differentiated cells. Disclosed are compositions comprising modified pluripotent cells wherein the pluripotent cells are modified to promote miR-155-5p strand selection in differentiated cells. In some embodiments, the differentiated cells are DA neurons.

Provided herein are genetically engineered mammalian stem and progenitor cells with increased potential to differentiate into DA neural cells wherein the DA neural cells have enhanced miR-155-3p strand selection compared to wildtype. Provided herein are genetically engineered mammalian stem and progenitor cells with increased potential to differentiate into DA neural cells wherein the DA neural cells have enhanced miR-155-3p strand selection compared to miR-155-5p strand selection. Provided herein are genetically engineered mammalian stem and progenitor cells with increased potential to differentiate into DA neural cells wherein the DA neural cells have enhanced miR-155-5p strand selection compared to wildtype. Provided herein are genetically engineered mammalian stem and progenitor cells with increased potential to differentiate into DA neural cells wherein the DA neural cells have enhanced miR-155-5p strand selection compared to miR-155-3p strand selection.

Provided herein is an isolated modified pluripotent cell comprising a disrupted pre-miR-155-3p, a disrupted pre-miR-155-5p, or both a disrupted pre-miR-155-3p and pre-miR-155-5p. An isolated modified pluripotent cell comprising a disrupted pre-miR-155-stem loop. An isolated modified pluripotent cell comprising a disrupted pre-miR-155-3p, pre-miR-155-5p and pre-miR-155-stem loop.

Provided herein is an isolated modified pluripotent cell comprising a disrupted pre-miR-155-3p, a disrupted pre-miR-155-5p, or both a disrupted pre-miR-155-3p and pre-miR-155-5p, wherein miR-155-3p strand selection is increased over wildtype miR-155-3p strand selection. An isolated modified pluripotent cell comprising a disrupted pre-miR-155-3p, a disrupted pre-miR-155-5p, or both a disrupted pre-miR-155-3p and pre-miR-155-5p, wherein miR-155-5p strand selection is increased over wildtype miR-155-5p strand selection.

Provided herein is a genetically engineered mammalian cell comprising a heterologous sequence in the genome, wherein the heterologous sequence comprises a transgene encoding a miR-155-3p strand selection agent, and wherein the miR-155-3p strand selection agent promotes the differentiation of the cell to a miR-155-3p enriched DA cell population or promotes the maintenance of the cell as a miR-155-3p enriched DA cell population. In some embodiments, the miR-155-3p strand selection agent is SEQ ID NO 7 or 8. In some embodiments, the miR-155-3p strand selection agent is SEQ ID NO 9-21. In some embodiments, the miR-155-3p strand selection agent is SEQ ID NO 6. In some embodiments, the engineered mammalian cell are differentiated using a mono-SMAD, or dual-SMAD method disclosed herein.

Provided herein is a genetically engineered mammalian cell comprising a heterologous sequence in the genome, wherein the heterologous sequence comprises a transgene encoding a miR-155-5p strand selection agent, and wherein the miR-155-5p strand selection agent promotes the differentiation of the cell to a miR-155-5p enriched DA cell population or promotes the maintenance of the cell as a miR-155-5p enriched DA cell population. In some embodiments, the miR-155-5p strand selection agent is SEQ ID Nos 6 or 8. In some embodiments, the miR-155-5p strand selection agent is SEQ ID NO 7. In some embodiments, the engineered mammalian cell are differentiated using a mono-SMAD, or dual-SMAD method disclosed herein.

Provided is a method of predisposing pluripotent cells to differentiate into miR-155-3p biased or miR-155-5p biased DA neurons, the method comprising up-regulating a level of at least one miRNA selected from the group comprising or consisting of miR-155-3p or miR-155-5p in the pluripotent cells, thereby predisposing the pluripotent cells to differentiate into the miR-155-3p biased or miR-155-5p biased DA neurons.

Provided is a method of predisposing pluripotent cells to differentiate into miR-155-3p biased DA neurons, the method comprising up-regulating a level of exogenous miR-155-3p in the pluripotent cells and down-regulating a level of miR-155-5p in the pluripotent cells, thereby predisposing the pluripotent cells to differentiate into miR-155-3p biased DA neurons.

Provided is a method of predisposing pluripotent cells to differentiate into miR-155-5p biased DA neurons, the method comprising up-regulating a level of exogenous miR-155-5p in the pluripotent cells and down-regulating a level of miR-155-3p in the pluripotent cells, thereby predisposing the pluripotent cells to differentiate into miR-155-5p biased DA neurons.

Provided is a method of predisposing pluripotent stem cells to differentiate into of miR-155-3p-biased DA neurons or of miR-155-5p DA neurons, the method comprising up-regulating a level of at least one exogenous miRNA selected from the group comprising or consisting of miR-155-3p or miR-155-5p in pluripotent stem cells, thereby predisposing the pluripotent stem cells to differentiate into the miR-155-3p-biased or miR-155-5p-biased DA neurons.

Provided is a genetically modified isolated population of cells which comprise at least one modified miRNA selected from the group comprising or consisting of miR-155-5p and miR-155-3p wherein the genetically modified cells have a pluripotent cell phenotype. Provided is a genetically modified isolated population of cells which comprise at least one modified miRNA selected from the group comprising or consisting of miR-155-5p and miR-155-3p and/or which comprise at least one polynucleotide agent that hybridizes and inhibits a function of at least one miRNA selected from the group comprising or consisting of miR-155-5p and miR-155-3p, wherein the genetically modified cells have a pluripotent cell phenotype.

Provided is a genetically modified isolated population of cells which comprise at least one modified miRNA selected from the group comprising or consisting of miR-155-5p and miR-155-3p wherein the genetically modified cells have a DA neural cell phenotype. Provided is a genetically modified isolated population of cells which comprise at least one modified miRNA selected from the group comprising or consisting of miR-155-5p and miR-155-3p and/or which comprise at least one polynucleotide agent that hybridizes and inhibits a function of at least one miRNA selected from the group comprising or consisting of miR-155-5p and miR-155-3p, wherein the genetically modified cells have a DA neural cell phenotype.

Provided is a genetically modified isolated population of cells which comprise at least one modified miRNA selected from the group comprising or consisting of miR-155-3p or miR-155-5p and/or which comprise at least one polynucleotide agent that hybridizes and inhibits a function of at least one miRNA selected from the group comprising or consisting of miR-155-3p or miR-155-5p. In some embodiments, genetically modified isolated population of cells are pluripotent cells. In some embodiments, genetically modified isolated population of cells are neural cells. In some embodiments, genetically modified isolated population of cells are DA neural cells.

Provided is a method of predisposing pluripotent cells to differentiate into miR-155-3p biased or miR-155-5p biased DA neurons, the method comprising up-regulating a level of at least one exogenous miRNA selected from the group comprising or consisting of miR-155-3p or miR-155-5p.

Provided a method of predisposing pluripotent cells to differentiate into miR-155-3p biased or miR-155-5p biased DA neurons, the method comprising down-regulating an expression of at least one miRNA selected from the group consisting of miR-155-3p or miR-155-5p.

Provided is a method of predisposing pluripotent cells to differentiate into miR-155-3p biased or miR-155-5p biased DA neurons, the method comprising contacting the iPSCs with an agent that down-regulates an amount and/or activity of miR-155-3p, thereby predisposing iPSC to differentiate into miR-155-3p biased or miR-155-5p biased DA neurons.

Provided is a method of predisposing pluripotent cells to differentiate into miR-155-3p biased or miR-155-5p biased DA neurons, the method comprising treating the iPSCs with an agent that down-regulates the amount and/or alters the activity of miR-155-5p, thereby predisposing iPSC to differentiate into miR-155-3p biased or miR-155-5p biased DA neurons.

As used herein, the phrase “predisposing pluripotent cells to differentiate into miR-155-3p biased or miR-155-5p biased DA neurons” refers to causing the pluripotent cells to differentiate along the neuronal lineage. The generated cells may be fully differentiated into DA neurons, or partially differentiated into DA neurons.

According to some embodiments, the pluripotent cells are isolated from a tissue selected from the group comprising bone marrow, adipose tissue, placenta, cord blood and umbilical cord.

According to some embodiments, the pluripotent cells are autologous to the subject. According to some embodiments, the pluripotent cells are non-autologous to the subject. According to some embodiments, the pluripotent cells are semi-allogeneic to the subject.

According to some embodiments, miR-155-3p biased or miR-155-5p biased DA neurons cells are produced by differentiating pluripotent cells. The miR-155-3p biased or miR-155-5p biased DA neurons cells are produced by introducing into the pluripotent the at least one modified miRNA. According to some embodiments, introduction into the pluripotent cells the at least one miRNA is affected by transfecting the pluripotent cell with an expression vector which comprises a polynucleotide sequence which encodes a pre-miRNA of the at least one miRNA. In some embodiments, the at least one miRNA is miR-155-3p or miR-155-5p and/or pre-miR-155-stem loop species.

According to some embodiments, introduction into the pluripotent the at least one miRNA is affected by transfecting the pluripotent cell with an expression vector which comprises a polynucleotide sequence which encodes the at least one miRNA. In some embodiments, the at least one miRNA is miR-155-3p or miR-155-5p and/or pre-miR-155-stem loop. In some embodiments, the at least one miRNA is selected from SEQ ID Nos 6-21.

According to some embodiments, the method further comprises analyzing an expression of at least one marker selected from the group consisting of FOXA2, LMX1A and TH following the generation of DA neurons.

### DA Neuronal Cell Populations

A DA neuronal cell population in which the level of miR-155-3p (5′CUCCUACAUAUUAGCAUUAACA3′) or a variant thereof is increased or decreased compared to wildtype. A DA neuronal cell population in which the level of miR-155-3p (5′CUCCUACAUAUUAGCAUUAACA3′) or a variant thereof is increased or decreased compared to miR-155-5p (5′UUAAUGCUAAUCGUGAUAGGGGUU3′).

A DA neuronal cell population in which the level of miR-155-3p (5′UUCCUACAUAUUAGCAUUAACA3′) or a variant thereof is increased or decreased compared to wildtype. A DA neuronal cell population in which the level of miR-155-3p (5′UUCCUACAUAUUAGCAUUAACA3′) or a variant thereof is increased or decreased compared to miR-155-5p (5′GGAAUGCUAAUCGUGAUAGGGGUU3′).

A DA neuronal cell population in which the level of miR-155-5p (5′UUAAUGCUAAUCGUGAUAGGGGUU3′) or a variant thereof is increased or decreased compared to wildtype. A DA neuronal cell population in which the level of miR-155-5p (5′UUAAUGCUAAUCGUGAUAGGGGUU3′) or a variant thereof is increased or decreased compared to miR-155-3p (5′UUCCUACAUAUUAGCAUUAACA3′).

A DA neuronal cell population in which the level of miR-155-5p (5′UUAAUGCUAAUCGUGAUAGGGGUU3′) or a variant thereof is increased or decreased compared to wildtype. A DA neuronal cell population in which the level of miR-155-5p (5′UUAAUGCUAAUCGUGAUAGGGGUU3′) or a variant thereof is increased or decreased compared to miR-155-3p (5′UUCCUACAUAUUAGCAUUAACA3′).

A DA neuronal cell population in which the level of miR-155-5p (5′GGAAUGCUAAUCGUGAUAGGGGUU3′) or a variant thereof is increased or decreased compared to miR-155-3p (5′CUCCUACAUAUUAGCAUUAACA3′).

A DA neuronal cell population in which the level of miR-155-3p (5′UUCCUACAUAUUAGCAUUAACA3′) or a variant thereof is increased or decreased compared to miR-155-5p (5′ UUAAUGCUAAUCGUGAUAGGGGUU3′).

A DA neuronal cell population in which the level of miR-155-5p (5′UUAAUGCUAAUCGUGAUAGGGGUU3′) or a variant thereof is increased or decreased compared to miR-155-3p (5′CUCCUACAUAUUAGCAUUAACA3′).

A DA neuronal cell population in which the level of miR-155-3p (5′CUCCUACAUAUUAGCAUUAACA3′) or a variant thereof is increased or decreased compared to miR-155-5p (5′UUAAUGCUAAUCGUGAUAGGGGUU3′).

A composition, comprising, an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-5p enhanced midbrain dopamine (DA) neurons. A composition, comprising, an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-5p modified midbrain dopamine (DA) neurons.

A composition, comprising, an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-3p modified midbrain dopamine (DA) neurons.

Disclosed is a composition comprising, an in vitro cell population comprising modified miR-155-3p pluripotent cells, and differentiation thereof to DA neurons to produce a miR-155-3p biased DA neuron cell population having an anti-inflammatory phenotype. In some embodiments, the cell population is differentiated using a mono-SMAD, or dual-SMAD method disclosed herein.

Disclosed is a composition comprising, an in vitro cell population comprising modified pre-miR-155 pluripotent cells, and differentiation thereof to DA neurons to produce a miR-155-5p biased DA neuron cell population having an pro-inflammatory phenotype. In some embodiments, the cell population is differentiated using a mono-SMAD, or dual-SMAD method disclosed herein.

In some embodiments, the DA neural cell population comprises three distinct cell populations: (1) A9 dopamine neurons (2) Astrocytes and (3) vascular leptomeningeal cells (VLMC).

In some embodiments, transfection of pluripotent cells at miR-155-3p and differentiation down the neuronal pathway produces miR-155-3p-biased dopamine neurons. In some embodiments, transfection of pluripotent cells at miR-155-3p and differentiation down the neuronal pathway produces miR-155-3p-biased Astrocytes. In some embodiments, transfection of pluripotent cells at miR-155-3p and differentiation down the neuronal pathway produces miR-155-3p-biased VLMC. In some embodiments, transfection of pluripotent cells at miR-155-3p and differentiation down the neuronal pathway (using a mono-SMAD, dual-SMAD or other approach) produces miR-155-3p-biased dopamine neurons, Astrocytes and VLMC. In some embodiments, transfection of pluripotent cells at miR-155-3p and differentiation down the neuronal pathway produces miR-155-3p-biased dopamine neurons, Astrocytes and VLMC wherein expression of miR-155-3p is greater in the dopamine neurons compared to the miR-155-3p-based Astrocytes and miR-155-3p-baised VLMC. In some embodiments, transfection of pluripotent cells at miR-155-3p and differentiation down the neuronal pathway produces miR-155-3p-biased dopamine neurons, Astrocytes and VLMC wherein expression of 155-3p is greater in the Astrocytes compared to the miR-155-3p-baised dopamine neurons and miR-155-3p-baised VLMC. In some embodiments, transfection of pluripotent cells at miR-155-3p and differentiation down the neuronal pathway produces miR-155-3p-biased dopamine neurons, Astrocytes and VLMC wherein expression of miR_155-3p is greater in the VLMC compared to the miR-155-3p-baised dopamine neurons and miR_155-3p-baised dopamine neurons.

In some embodiments, transfection of pluripotent cells at miR-155-5p and differentiation down the neuronal pathway produces miR-155-5p-biased dopamine neurons. In some embodiments, transfection of pluripotent cells at miR-155-5p and differentiation down the neuronal pathway produces miR-155-5p-biased Astrocytes. In some embodiments, transfection of pluripotent cells at miR-155-5p and differentiation down (using a mono-SMAD, dual-SMAD or other approach) the neuronal pathway produces miR-155-5p-biased VLMC. In some embodiments, transfection of pluripotent cells at miR-155-5p and differentiation down the neuronal pathway produces miR-155-5p-biased dopamine neurons, Astrocytes and VLMC. In some embodiments, transfection of pluripotent cells at miR-155-5p and differentiation down the neuronal pathway produces miR-155-5p-biased dopamine neurons, Astrocytes and VLMC wherein expression of miR-155-5p is greater in the dopamine neurons compared to the miR-155-5p-baised Astrocytes and miR-155-5p-biased VLMC. In some embodiments, transfection of pluripotent cells at miR-155-5p and differentiation down the neuronal pathway produces miR-155-5p-biased dopamine neurons, Astrocytes and VLMC wherein expression of miR-155-5p is greater in the Astrocytes compared to the miR-155-5p-baised dopamine neurons and miR-155-5p-biased VLMC. In some embodiments, transfection of pluripotent cells at miR-155-5p and differentiation down the neuronal pathway produces miR-155-5p-biased dopamine neurons, Astrocytes and VLMC wherein expression of miR-155-5p is greater in the VLMC compared to the miR-155-5p-biased dopamine neurons and miR-155-5p-biased dopamine neurons.

In some embodiments, transfection of pluripotent cells is at a genomic safe harbor or a transcriptional cassette whereby a pol II promoter is driving expression of a mini-gene and whose primary transcript contains a pre-mir-155 of native sequence, changed sequence to yield miR-155-3p-biased or to yield a miR-155-5p-biased cell population.

### Cell Culture

Another aspect of the present disclosure relates to a culture of modified pluripotent cells modified by a method as described above. The culture may be comprised in a container means. The pluripotent cells may be comprised in a pharmaceutical preparation such as, e.g., a pharmaceutical preparation formulated for injection.

Another aspect of the present disclosure relates to a culture of modified midbrain dopaminergic (DA) neurons generated by a method described above. The culture may be comprised in a container means. The neurons may be comprised in a pharmaceutical preparation such as, e.g., a pharmaceutical preparation formulated for injection.

### Diagnostic Methods

In some embodiments, disclosed is a method of screening a test compound comprising: (a) contacting the test compound with miR-155-5p or miR-155-3p enhanced DA neural cells, and (b) measuring the function, physiology, or viability of the cells. In some embodiments, said measuring comprises testing for a toxicological response or an altered electrophysiological response of the cells.

### Therapeutic Methods

Provided is a method of treating a brain disease or disorder in a subject in need thereof, the method comprising administering to the subject a therapeutically effective amount of the isolated population of cells comprising miR-155-3p biased or miR-155-5p biased cells, thereby treating the brain disease or disorder. In some embodiments, the miR-155-3p biased or miR-155-5p biased isolated population of cells are pluripotent cells. In some embodiments, the miR-155-3p biased or miR-155-5p biased isolated population of cells are neural cells. In some embodiments, the miR-155-3p biased or miR-155-5p biased isolated population of cells are DA neural cells. In some embodiments, the miR-155-3p biased or miR-155-5p biased isolated population of cells are A9 dopamine neurons. In some embodiments, the miR-155-3p biased or miR-155-5p biased isolated population of cells are astrocytes. In some embodiments, the miR-155-3p biased or miR-155-5p biased isolated population of cells are VLMC.

In some embodiments, disclosed is a method of treating a disease in a mammalian subject comprising administering to the subject a therapeutically effective amount of miR-155-5p or miR-155-3p biased DA neural cells. The mammalian subject may be a human. The disease may be a disease of the central nervous system (CNS). In some embodiments, the disease is a Parkinsonian disorder, Parkinson's disease (PD) or a Parkinson-plus syndrome (PPS). In some embodiments, the disease is multiple sclerosis, epilepsy, amyotrophic lateral sclerosis (ALS), stroke, Rett Syndrome, autoimmune encephalomyelitis, spinal cord injury, cerebral palsy, stroke, Alzheimer's disease or Huntingdon's disease. According to some embodiments, the method of treating a disease is for use in treating a neurological disease.

In one embodiment, provided is a method of engrafting cells in vivo for therapeutic treatment, comprising, a) providing: i) a population of midbrain dopamine (DA) neurons wherein miR-155-5p is expressed more than miR-155-3p; and ii) a subject, wherein said subject shows at least one neurological symptom; and b) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons wherein miR-155-3p is expressed more than miR-155-5p); and ii) a subject, wherein said subject shows at least one neurological symptom; and b) transplanting said midbrain dopamine (DA) neurons into said subject under conditions for allowing in vivo engraftment and for providing dopamine (DA) neuronal function. In one embodiment, said neurological symptoms are selected from the group consisting of tremor, bradykinesia (extreme slowness of movement), flexed posture, postural instability and rigidity. In one embodiment, said subject shows reduction of said neurological symptom. In one embodiment, said population of midbrain dopamine (DA) neurons are derived from a cell population selected from the group comprising primates and humans. In one embodiment, said human cells are cells from a patient with a symptom of Parkinson's disease (PD).

In one aspect disclosed is an in vitro cell population having a unique molecular profile in that the microRNA (miR or miRNA) and/or the miRNA profile of the cell population comprises more miR-155-3p compared to miR-155-5p or more modified miR-155-3p compared to un-modified miR-155-3p or more modified miR-155-5p compared to un-modified miR-155-5p. In one aspect disclosed is an in vitro cell population of having a unique molecular profile in that the microRNA (miR or miRNA) and/or the miRNA profile of the cell population comprises more miR-155-5p compared to miR-155-3p. In one aspect disclosed is an in vitro cell population of having a unique molecular profile in that the microRNA (miR or miRNA) and/or the miRNA profile of the cell population comprises more miR-155-3p compared to miR-155-5p. In one aspect the in vitro cell compositions comprise, or alternatively consist essentially of, or yet further consist of, one or more, two or more, three or more, four or more, five or more, six or more, seven or more, eight or more, nine or more, ten or more, eleven or more, twelve or more, thirteen or more, fourteen or more, fifteen or more, sixteen or more, twenty or more, thirty or more, forty or more, fifty or more, sixty or more, seventy or more, eighty or more, ninety or more, ninety-five or more or ninety-nine or more times more miR-155-3p than miR-155-5p. In one aspect the in vitro cell compositions comprise, or alternatively consist essentially of, or yet further consist of, one or more, two or more, three or more, four or more, five or more, six or more, seven or more, eight or more, nine or more, ten or more, eleven or more, twelve or more, thirteen or more, fourteen or more, fifteen or more, sixteen or more, twenty or more, thirty or more, forty or more, fifty or more, sixty or more, seventy or more, eighty or more, ninety or more, ninety-five or more or ninety-nine or more times more miR-155-5p than miR-155-3p. In some embodiments, in vitro cell population is pluripotent cells. In some embodiments, the in vitro cell population is DA neurons. In some embodiments, the in vitro cell population is A9 dopamine neurons, astrocytes or vascular leptomeningeal cells (VLMC). These compositions are useful for the treatment of disease, such as neurologic disease, Parkinson's disease and associated disorders, including but not limited to Parkinsonian disorders.

This disclosure also provides a pharmaceutical compositions comprising, or alternatively consisting essentially of, or yet further consisting of, a pharmaceutically acceptable carrier and an effective amount of cells differentiated from pluripotent cells. In one aspect, the differentiated cell population has a unique molecular profile in that the microRNA (miR) comprises more miR-155-3p compared to miR-155-5p or more modified miR-155-3p compared to un-modified miR-155-3p or more modified miR-155-5p compared to un-modified miR-155-5p. In one aspect, the differentiated cell population has a unique molecular profile in that the microRNA (miR) comprises more miR-155-3p compared to wildtype. In one aspect, the differentiated cell population has a unique molecular profile in that the microRNA (miR) comprises more miR-155-5p compared to miR-155-3p. In one aspect, the differentiated cell population has a unique molecular profile in that the microRNA (miR) comprises more miR-155-5p compared to wildtype. In one aspect, the differentiated cell population has a unique molecular profile in that the microRNA (miR) comprises more miR-155-3p compared to miR-155-5p. In one aspect, the differentiated cell population has a unique molecular profile in that the microRNA (miR) comprises more miR-155-5p compared to wildtype. In some embodiments, the cells differentiated from pluripotent cells are DA neurons. In some embodiments, the cells differentiated from pluripotent cells are A9 dopamine neurons, astrocytes or vascular leptomeningeal cells (VLMC).

In further aspect, the cell populations of the above-noted compositions are identified by the microRNA (miR) profile by the lack of up-regulation of miR-155-5p compared to miR-155-3p. In further aspect, the cell populations of the above-noted compositions are identified by the microRNA (miR) profile by the lack of up-regulation of miR-155-3p compared to miR-155-5p.

For each of the above compositions, the compositions can be administered to subjects identified as likely to have a neurologic disease such as Parkinson's disease.

The compositions are useful for the preparation of a medicament and/or to perform methods for one or more of: a) inhibiting the progression of, b) preventing or c) treating, Parkinson's disease or an associated disorder in a subject in need thereof. The methods comprise, or alternatively consist essentially of, or yet further consist of, administering to the subject an effective amount of the pharmaceutical composition described above. Non-limiting examples of an associated disorder is selected from the group of: Huntington's, Alzheimer's, and multiple sclerosis. These conditions are well known in the art and can be diagnosed by a treating physician.

The therapy and patient's health can be monitored by determining the level of inflammatory response, during and after the therapy.

Also provided herein are isolated or purified cell populations isolated from a body fluid (e.g., urine, saliva, lymphatic fluid, breast milk, blood, serum and/or plasma) of a non-diseased subject or differentiated from an iPSC. In one aspect, the cell populations have a unique molecular profile in that the microRNA (miR) profile in the cell populations comprise the up-regulation of miR-155-3p or the up-regulation of miR-155-3p compared to miR-155-5p, or the up-regulation of miR-155-3p compared to wildtype.

Also provided herein are isolated or purified cell populations isolated from a body fluid (e.g., urine, saliva, lymphatic fluid, breast milk, blood, serum and/or plasma) of a non-diseased subject or differentiated from an iPSC. In one aspect, the cell populations have a unique molecular profile in that the microRNA (miR) profile in that the cell populations comprise the up-regulation of miR-155-5p or the up-regulation of miR-155-5p compared to miR-155-3p, or the up-regulation of miR-155-5p compared to wildtype.

Provided is a pharmaceutical composition comprising the isolated population of cells described herein and a pharmaceutically acceptable carrier. In some embodiments, the isolated population of cells comprise miR-155-3p biased or miR-155-5p biased cell population. In some embodiments, the miR-155-3p biased or miR-155-5p biased isolated population of cells are pluripotent cells. In some embodiments, the miR-155-3p biased or miR-155-5p biased isolated population of cells are neural cells. In some embodiments, the miR-155-3p biased or miR-155-5p biased isolated population of cells are DA neural cells. In some embodiments, the miR-155-3p biased or miR-155-5p biased isolated population of cells are A9 dopamine neurons, astrocytes or vascular leptomeningeal cells (VLMC).

Provided is a method of treating a neurological disease in a subject in need thereof, the method comprising administering to the subject a therapeutically effective amount of the isolated population, thereby treating the brain disease or disorder. In some embodiments, the isolated population of cells comprise miR-155-3p biased or miR-155-5p biased cell population. In some embodiments, the miR-155-3p biased or miR-155-5p biased isolated population of cells are pluripotent cells. In some embodiments, the miR-155-3p biased or miR-155-5p biased isolated population of cells are neural cells. In some embodiments, the miR-155-3p biased or miR-155-5p biased isolated population of cells are DA neural cells.

According to some embodiments, the isolated population of cells is for use in treating a brain disease or disorder. According to some embodiments, the brain disease or disorder is a neurodegenerative disorder. According to some embodiments, the neurodegenerative disorder is selected from the group consisting of Parkinsonian disorders, multiple sclerosis, Parkinson's disease, epilepsy, amyotrophic lateral sclerosis (ALS), stroke, Rett Syndrome, autoimmune encephalomyelitis, spinal cord injury, cerebral palsy, stroke, Alzheimer's disease and Huntingdon's disease. According to some embodiments, the isolated population is for use in treating a neurological disease.

### Kits

A kit comprising compounds necessary to modify pre-miR-155-5p or pre-miR-155-3p DA neurons. A kit comprising compounds necessary to differentiate pluripotent cells to miR-155-5p or miR-155-3p DA neurons. A kit comprising compounds necessary to differentiate pluripotent cells to miR-155-5p biased or miR-155-3p biased DA A9 dopamine neurons, astrocytes and vascular leptomeningeal cells (VLMC).

A kit also is provided for one or more of: a) inhibiting the progression of, b) preventing or c) treating, Parkinson's disease or an associated disorder in a subject in need thereof, comprising an effective amount of the isolated or purified cell populations and/or the pharmaceutical composition as described above and/or reagents and/or instructions for use.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein the term “about” refers to +/−10%.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “activator” or “activating” refers to small molecules, peptides, proteins and compounds for activating molecules resulting in directed differentiation of modified pluripotent cells herein described. Exemplary activators include but are not limited to: CHIR, Sonic hedgehog (SHH) C25II, a small molecule Smoothened agonist purmorphamine, fibroblast growth factor (FGF), etc.

As used herein, the term “activator of Sonic hedgehog (SHH) signaling” refers to any molecule or compound that activates a SHH signaling pathway including a molecule or compound that binds to PCT or a Smoothened agonist and the like. Examples of such compounds are a protein Sonic hedgehog (SHH) C25II and a small molecule Smoothened agonist purmorphamine.

The term “Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, the disease being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application.

An agent of the present disclosure can be administered for therapy by any suitable route of administration. It will also be appreciated that the preferred route will vary with the condition and age of the recipient, and the disease being treated.

The term “aggregates,” i.e., embryoid bodies, refers to homogeneous or heterogeneous clusters of cells comprising differentiated cells, partly differentiated cells and/or pluripotent stem cells cultured in suspension.

As used herein, the term “cell” refers to a single cell as well as to a population of (i.e., more than one) cells. The population may be a pure population comprising one cell type, such as a population of neuronal cells or a population of undifferentiated embryonic cells. Alternatively, the population may comprise more than one cell type, for example a mixed cell population. It is not meant to limit the number of cells in a population; for example, in one embodiment, a mixed population of cells may comprise at least one differentiated cell. In some embodiments, there is no limit on the number of cell types that a cell population may comprise.

As used herein, the term “CHIR99021” or “CHIR” or “aminopyrimidine” or “3-[3-(2-Carboxyethyl)-4-methylpyrrol-2-methylidenyl]-2-indolinone” refers to IUPAC name 6-(2-(4-(2,4-dichlorophenyl)-5-(4-methyl-1H-imidazol-2-yl)pyrimidin-2-ylamino)ethylamino) nicotinonitrile. CHIR99021 is one example of a small-molecule chemical inhibitor of glycogen synthase kinase 3β (GSK3β) that activates a WNT signaling pathway, and is highly selective, showing nearly thousand fold selectivity against a panel of related and unrelated kinases, with an IC₅₀=6.7 nM against human GSK3β and nanomolar IC₅₀ values against rodent GSK3β homologs.

“DA neuronal cells” or “DA neurons” are cells of the midbrain fate that express FOXA2+, LMX1A+, and TH+.

As used herein, the term “differentiation” refers to a process whereby an unspecialized embryonic cell acquires the features of a specialized cell such as a specific type of neuron, brain cell, heart, liver, or muscle cell. Differentiation is controlled by the interaction of a cell's genes with the physical and chemical conditions outside the cell, usually through signaling pathways involving proteins embedded in the cell surface.

As used herein, the term “cell differentiation” refers to a pathway by which a less specialized cell (i.e. stem cell) develops or matures to possess a more distinct form and function (for example, an iPSC progressing into a neural crest progenitor to a cell of neuronal lineage to a floor plate midbrain progenitor cells to a midbrain fate FOXA2/LMX1A+ dopamine (DA) neurons).

The term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vivo, treatment and/or mutagenesis. A cell may be derived from another cell, using for example chemical treatment, radiation, inducing new protein expression, for example, by infection with virus, transfection with DNA/RNA sequences, contacting (treating) with a morphogen, etc., and selection (such as by serial culture) of any cell type that is contained in cultured parent cells). A derived cell can be selected from a mixed population by virtue of response to a growth factor, cytokine, selected progression of cytokine treatments, adhesiveness, lack of adhesiveness, sorting procedure, and the like.

As used herein, the term “directed differentiation” refers to a manipulation of stem cell culture conditions to induce differentiation into a particular (for example, desired) cell type, such as floor plate midbrain progenitor cells and midbrain fate FOXA2/LMX1A+ dopamine (DA) neurons. In one embodiment, the tam “directed differentiation” in reference to a cell refers to the use of small molecules, growth factor proteins, and other growth conditions to promote the transition of a cell from a pluripotent state into a more mature or specialized cell fate (e.g. central nervous system cell, neural cell, floor plate midbrain progenitor cell and midbrain fate FOXA2/LMX1A+ dopamine (DA) neuron, etc.). In one preferred embodiment, the beginning of directed differentiation is the contacting of a cell at day 0 with LDN/SB. A cell undergoing directed differentiation as described herein results in the formation of a non-default cell type of floor plate midbrain progenitor cells and midbrain fate FOXA2/LMX1A+ dopamine (DA) neurons.

As used herein, the term “dopamine neuron” or “dopaminergic neuron” in general refers to a cell capable of expressing dopamine. “Midbrain dopamine neurons” or “mDA” refer to presumptive dopamine expressing cells in forebrain structures and dopamine expressing cells in forebrain structures.

The terms “Complementary DNA” or “cDNA” means a single-stranded or double-stranded DNA that contains a sequence complementary to an mRNA sequence and does not contain any intronic sequence.

The term “Antisense” means a nucleic acid molecule complementary to the respective mRNA molecule. The antisense conformation is indicated as a “−” or “*” symbol or with an “a” or “antisense” in front of the DNA or RNA, e.g., “aDNA” or “aRNA”.

The terms “Base Pair” or “bp” means a partnership of adenine (A) with thymine (T), or of cytosine (C) with guanine (G) in a double stranded DNA molecule. In RNA, uracil (U) is substituted for thymine. Generally the partnership is achieved through hydrogen bonding. For example, a sense nucleotide sequence “5′-A-T-C-G-U-3” can form complete base pairing with its antisense sequence “5′-A-C-G-A-T-3”. Also, G and U may form non-Watson-and-Crick pairing, such as “5′-T-G-C-3” pairing with “5′-G-U-A-3”.

The term “Conserved” means a nucleotide sequence is conserved with respect to a pre-selected (referenced) sequence if it non-randomly hybridizes to an exact complement of the pre-selected sequence.

The terms “Complementary” or “Complementarity” or “Complementation” is a term used in reference to matched base pairing between two polynucleotides (i.e. sequences of an mRNA and a cDNA) related by the aforementioned “base pair (bp” rules. For example, the sequence “5′-A-G-T-3′” is complementary to the sequence “5′-A-C-T-3”, and also to “5′-A-C-U-3”. Also, G and U may be complementary to each other in an RNA duplex or RNA-DNA pairing sequence. For example, the sequence “5′-U-G-C-3” is complementary to the sequence “5′-G-U-A-3”, and also to “5′-G-U-G-3′” as well as to “5′-G-C-G-3” and “5′-G-C-A-3”. Complementation can be between two DNA strands, a DNA and an RNA strand, or between two RNA strands. Complementarity may be “partial” or “complete” or “total”. Partial complementarity or complementation occurs when only some of the nucleic acid bases are matched according to the base pairing rules. Complete or total complementarity or complementation occurs when the bases are completely or perfectly matched between the nucleic acid strands. The degree of complementarity between nucleic acid strands has significant effects on the efficiency and strength of hybridization between nucleic acid strands. This is of particular importance in amplification reactions, as well as in detection methods that depend on binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

The term “Complementary Bases” means nucleotides that normally pair up when DNA or RNA adopts a double stranded configuration, such as DNA-DNA, DNA-RNA, and RNA-RNA duplexes as well as any duplex formed by pairing between partial DNA and partial RNA hybrid sequences.

The term “Complementary Nucleotide Sequence” means a sequence of nucleotides in a single-stranded molecule of DNA or RNA that is sufficiently complementary to that on another single strand to specifically hybridize between the two strands with consequent hydrogen bonding.

As used herein, the term “derivative” refers to a chemical compound with a similar core structure.

The term “Differentiation” means a process by which a less specialized cell forms progeny of at least a new cell type which is more specialized.’

The term “differentiation day” refers to the day of incubation of cells in a media, wherein initiation of exposure of pluripotent cells to a differentiation media on day 1. In some preferred embodiments, the differentiation media on day 1 includes a single SMAD inhibitor. Prior to incubation or culture in a differentiation media, cells may be incubated, e.g., for 1, 2, or 3 days prior to incubation in the differentiation media (i.e., on day 0, day −1, and/or day −2) in a medium comprising or consisting of Essential 8™ Basal Medium and Essential 8™ Supplement (Thermo Fisher Scientific; Waltham, Mass.), optionally with the addition of a ROCK inhibitor (e.g., inclusion of about 0.25-5 μM, 0.5, 0.75, 1, 1.25, 1.5, 2, 3, 4, or any range derivable therein of H1152, e.g., on day-2), and/or blebbestatin (e.g., at a concentration of about 0.1-20 μM, more preferably about 1.25-5 μM, or about 2.5 μM).

The term “An effective amount” intends to indicate the amount of a composition, compound or agent (cell populations) administered or delivered to the subject that is most likely to result in the desired response to treatment. The amount is empirically determined by the patient's clinical parameters including, but not limited to the stage of disease, age, gender and histology.

The term “Embryonic stem (ES) cells” means pluripotent stem cells derived from early embryos. The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

The term “Essentially free” refers to a medium that does not have, or that have essentially none of, the specified component from a source other than the cells in the medium. “Essentially free” of externally added growth factors or signaling inhibitors, such as TGFβ, bFGF, TGFβ superfamily signaling inhibitors, etc., may mean a minimal amount or an undetectable amount of the externally added component. For example, a medium or environment essentially free of TGFβ or bFGF can contain less than 5, 4, 3, 2, 1, 0.9, 0.8, 0.7, 0.6, 0.5, 0.4, 0.3, 0.2, 0.1, 0.01, 0.001 ng/mL or any range derivable therein. For example, a medium or environment essentially free of signaling inhibitors can contain less than 0.2, 0.1, 0.09, 0.08, 0.07, 0.06, 0.05, 0.04, 0.03, 0.02, 0.01, 0.005, 0.001 μM, or any range derivable therein.

The term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “Exon” means a part or parts of a gene transcript sequence encoding protein-reading frames (cDNA), such as cDNA for cellular genes, growth factors, insulin, antibodies and their analogs/homologs as well as derivatives.

The term “Expression” as applied to a gene, refers to the differential production of the miR or mRNA transcribed from the gene or the protein product encoded by the gene. A differentially expressed gene may be over expressed (high expression) or under expressed (low expression) as compared to the expression level of a normal or control cell, a given patient population or with an internal control gene (housekeeping gene). In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample. If the expression level of a gene of interest for the single patient is determined to be above the median expression level of the patient population, that patient is determined to have high expression (up-regulated) of the gene of interest. Alternatively, if the expression level of a gene of interest for the single patient is determined to be below the median expression level (down-regulated) of the patient population, that patient is determined to have low expression of the gene of interest.

As used herein, the term “floor plate midbrain progenitor cell” in reference to an in vivo cell located in a midbrain, including during embryonic development of midbrain neurons, refers to a cell that may differentiate into a dopamine producing cell. In some embodiments, a “floor plate midbrain progenitor cell” refers to a cell in culture that is used to artificially produce a cultured cell in vitro that expresses overlapping or identical sets of markers when compared to markers expressed by in vivo cells, i.e. co-expression of the floor plate marker FOXA2 and the roof plate marker LMX1A, OTX2, NGN2, and DDC, such as in cultured cells disclosed herein around day 11 after initiation of directed differentiation as described herein. Preferably, a floor plate midbrain progenitor cell is “FOXA2+LMX1A+” or “FOXA2/LMX1A+”. In some embodiments, low numbers of cells in a differentiated progenitor population are FOXA2/LMX1A/TH+.

As used herein, the term “floor-plate derived DA neurons” or “authentic midbrain DA neurons” or “midbrain fate FOXA2+LMX1A+ dopamine (DA) neurons” or “floor plate midbrain dopamine (DA) neuron” or “engraftable midbrain DA neuron” or “mDA neuron” or “FOXA2+LMX1A+TH+” or “FOXA2/LMX1A/TH” or “FOXA2+LMX1A+NURR1+TH+” or “FOXA2/LMX1A/NURR1/TH” refers to an engraftable midbrain DA neuron population obtained by methods described herein, typically around or by day 25 after initiating directed differentiation. In a preferred embodiment, “authentic midbrain DA neurons” are FOXA2+/LMX1A+/NURR1+/TH+. These neurons were labeled “engraftable” after transplantation experiments in mice and primates showing the capability of these neurons to reverse Parkinson-like neurological conditions with less interference from neural overgrowth and teratoma formation. The midbrain fate FOXA2/LMX1A+ dopamine (DA) neurons can be maintained in vitro for several months while retaining engrafting capability.

As used herein, cells used for obtaining floor plate midbrain progenitor cells and midbrain fate FOXA2/LMX1A+ dopamine (DA) neurons are obtained from a variety of sources including embryonic and nonembryonic sources, for example, hESCs and nonembryonic hiPSCs (Human induced pluripotent stem cells), somatic stem cells, disease stem cells, i.e. isolated pluripotent cells and engineered derived stem cells isolated from Parkinson disease patients, cancer stem cells, human or mammalian pluripotent cells, etc.

The term “Gene” means a nucleic acid composition whose oligonucleotide or polynucleotide sequence codes for an RNA and/or a polypeptide (protein). A gene can be either RNA or DNA. A gene may encode a non-coding RNA, such as small hairpin RNA (shRNA), microRNA (miRNA), rRNA, tRNA, snoRNA, snRNA, and their RNA precursors as well as derivatives. Alternatively, a gene may encode a protein-coding RNA essential for protein/peptide synthesis, such as messenger RNA (mRNA) and its RNA precursors as well as derivatives. In some cases, a gene may encode a protein-coding RNA that also contains at least a microRNA or shRNA sequence.

The term “Gene Delivery” means a genetic engineering method selected from the group comprising polysomal transfection, liposomal transfection, chemical transfection, electroporation, viral infection, DNA/RNA recombination, transposon insertion, jumping gene insertion, microinjection, gene-gun penetration, and a combination thereof.

As used herein Gene Locus (MIR-155HG or BIC) which be native, i.e, not edited or edited. When edited the transcription of the pre-MIR-155 could result in processing to enhance miR-155-3p or miRNA-155-5p.

The term “Genetic Engineering” means a DNA/RNA recombination method selected from the group comprising DNA/RNA restriction and ligation, homologous recombination, transgene incorporation, transposon insertion, jumping gene integration, retroviral infection, and a combination thereof.

As used herein, the term “glycogen synthase kinase 3β inhibitor” or “GSK3β inhibitor” refers to a compound that inhibits a glycogen synthase kinase 3β enzyme, for example, see, Doble, et al., J Cell Sci. 2003; 116:1175-1186, herein incorporated by reference. A GSK3β inhibitor is capable of activating a WNT signaling pathway, see, for example, Cadigan, et al., J Cell Sci. 2006; 119:395-402; Kikuchi, et al., Cell Signaling. 2007; 19:659-671, herein incorporated by reference.

As used herein, the term “highly enriched population” refers to a population of cells, such as a population of cells in a culture dish, expressing a marker at a higher percentage or amount than a comparison population, for example, treating a LSB contacted cell culture on day 1 with purmorphamine and on day 3 with CHIR results in a highly enriched population of floor plate midbrain progenitor cells compare to treatment with LSB alone. In other examples, an enriched population is a population resulting from sorting or separating cells expressing one or more markers from cells not expressing the desired marker, such as a CD142 enriched population, an A9 enriched population, and the like.

The terms “Homologous” or “Homology” are terms indicating the similarity between a polynucleotide and a gene or mRNA sequence. A nucleic acid sequence may be partially or completely homologous to a particular gene or mRNA sequence, for example. Homology may be expressed as a percentage determined by the number of similar nucleotides over the total number of nucleotides. Also, thymine (T) and uracil (U) are homologous to each other. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

The terms “Hybridize” or “Hybridization” means the formation of duplexes between nucleotide sequences which are sufficiently complementary to form complexes via base pairing. Where a primer (or splice template) “hybridizes” with target (template), such complexes (or hybrids) are sufficiently stable to serve the priming function required by a DNA/RNA polymerase to initiate DNA/RNA synthesis. There is a specific, i.e. non-random, interaction between two complementary polynucleotides that can be competitively inhibited.

The term “Induced pluripotent stem cells” commonly abbreviated as “iPS cells” or “iPSCs,” refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting with reprogramming factors. Induced pluripotent stem cells are endowed with pluripotency (i.e., being capable of differentiating into the three embryonic germ cell layers, i.e., endoderm, ectoderm and mesoderm). According to some embodiments, the induced pluripotent stem cells are formed by inducing the expression of Oct-4.

As used herein, the term “inhibitor” or “signaling inhibitor” is in reference to inhibiting a signaling molecule or a signaling molecule's pathway, such as an inhibitor of SMAD signaling, inhibitor of glycogen synthase kinase 3β (GSK3β), refers to a compound or molecule (e.g., small molecule, peptide, peptidomimetic, natural compound, protein, siRNA, anti-sense nucleic acid, aptamer, or antibody) that interferes with (i.e. reduces or suppresses or eliminates or blocks) the signaling function of the molecule or pathway. In other words, an inhibitor is any compound or molecule that changes any activity of a named protein (signaling molecule, any molecule involved with the named signaling molecule, a named associated molecule, such as a glycogen synthase kinase 3β (GSK3β)) (e.g., including, but not limited to, the signaling molecules described herein), for one example, via directly contacting SMAD signaling, contacting SMAD mRNA, causing conformational changes of SMAD, decreasing SMAD protein levels, or interfering with SMAD interactions with signaling partners (e.g., including those described herein), and affecting the expression of SMAD target genes (e.g. those described herein). Inhibitors also include molecules that indirectly regulate SMAD biological activity by intercepting upstream signaling molecules. Thus in one embodiment, an inhibitor induces (changes) or alters differentiation from a default to a non-default cell type, for example, one of the methods disclosed herein comprising LDN/SB, CHIR and a SHH activator (which may inhibit glycogen synthase kinase 3β) differentiated progenitor cells into non-default neural progenitor cells. An inhibitor “alters” or “lowers” or “blocks” default signaling in order to direct cellular differentiation towards a nondefault cell type, such as described herein for differentiating floor plate midbrain progenitor cells and midbrain fate FOXA2/LMX1A+ dopamine (DA) neurons. Thus, an inhibitor is a natural compound or small molecule which changes signal molecule activity in a manner that contributes to differentiation of a starting cell population (day 0) into floor plate midbrain progenitor cells. When progenitor cells are contacted with inhibitors these small molecules may contribute to further differentiation into midbrain fate FOXA2/LMX1A+ dopamine (DA) neurons. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. In some cases, an inhibitor is referred to as a “direct inhibitor” which refers to inhibiting a signaling target or a signaling target pathway by actually contacting the signaling target; for example, a direct inhibitor of a gamma secretase is a DAPT molecule that binds to the gamma secretase protein.

The term “Intron” means a part or parts of a gene transcript sequence encoding non-protein-reading frames, such as in-frame intron, 5′-UTR and 3′-UTR.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA/RNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “kit” refers to any delivery system for delivering materials. In the context of cell differentiation, a kit may refer to a combination of materials for contacting stem cells, such delivery systems include systems that allow for the storage, transport, or delivery of reaction reagents from one location to another in the appropriate containers (such as tubes, etc.) and/or supporting materials (e.g., buffers, written instructions for performing cell differentiation, etc.) (e.g., compounds, proteins, detection agents (such as antibodies that bind to tyrosine hydroxylase (TH), forkhead box protein A2 (FOXA2), LIM homeobox transcription factor 1, alpha (LMX1A), etc.), etc. For example, kits include one or more enclosures (e.g., boxes, or bags, test tubes, Eppendorf tubes, capillary tubes, multiwell plates, and the like) containing relevant reaction reagents for inhibiting signaling pathways, for example, an inhibitor for lowering transforming growth factor beta (TGFβ)/Activin-Nodal signaling, such as SB431542 (or SB431542 replacement), and the like, an inhibitor for lowering SMAD signaling, LDN-193189 (or LDN-193189 replacement), and the like, an inhibitor for lowering glycogen synthase kinase 3β (GSK3β), for one example, for activation of wingless (Wnt or Wnts) signaling otherwise known as a WNT signaling activator (WNT agonist), such as CHIR99021 (or CHIR99021 replacement), etc.), and the like, an activator of Sonic hedgehog (SHH) signaling (such as a smoothened (SMO) receptor small molecule agonist), for example, a Sonic hedgehog (SHH) C25II molecule, purmorphamine, and the like, a molecule with Fibroblast growth factor 8 (FGF8) activity, such as Fibroblast growth factor 8 (FGF8), etc., and neuronal maturation molecules, for example, brain-derived neurotrophic factor (BDNF), ascorbic acid (AA), glial cell line-derived neurotrophic factor, dibutyryl cAMP and transforming growth factor type β3, including molecules capable of replacing these components, and/or supporting materials. The reagents in the kit in one embodiment may be in solution, may be frozen, or may be lyophilized. The reagents in the kit in one embodiment may be in individual containers or provided as specific combinations, such as a combination of LSB (LDN-193189 with SB431542), Sonic hedgehog (SHH) C25II molecule with purmorphamine, Sonic hedgehog (SHH) C25II molecule with purmorphamine with CHIR99021 or purmorphamine with CHIR99021, neuronal maturation molecules and the like.

The term “lack of up-regulation” intends and lack of “down-regulation” intends that the microRNA marker was not determined to be over- or under-expressed as compared to a predetermined value. In one aspect, the predetermined value is a preliminary value from the subject prior to the subsequent measurement (as in prior to therapy) or is a value from a population of subjects that do or do not have clinical manifestation of the related disorder. For example, when the disclosure relates to the treatment of Parkinson's disease, a predetermined value can be the average or median exosome miRNA value as measured from a population of subjects that do or do not have a fibrotic or hepatic disease or an associated disorder.

As used herein, the term “LDN-193189” refers to a small molecule DM-3189, IUPAC name 4-(6-(4-(piperazin-1-yl)phenyl) pyrazolo[1,5-a]pyrimidin-3-yl) quinoline, with a chemical formula of C₂₅H₂₂N₆. LDN-193189 is capable of functioning as a SMAD signaling inhibitor. LDN-193189 is also a highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18:4388-4392, herein incorporated by reference).

As used herein, the term “LSB” refers to a combination of two compounds LDN-193189 and SB431542 capable of lowering or blocking signaling consisting of transforming growth factor beta (TGFβ)/Activin-Nodal signaling and Small Mothers Against Decapentaplegic (SMAD) signaling in a cell.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The terms “Messenger RNA” or “mRNA” means an assembly of pre-mRNA exons, which is formed after intron removal by intracellular RNA splicing machineries (spliceosomes) and served as a protein-coding RNA for peptide/protein synthesis. The peptides/proteins encoded by mRNAs include, but not limited, enzymes, growth factors, insulin, antibodies and their analogs/homologs as well as derivatives.

The terms “MicroRNA” or “miRNA” means a single-stranded RNA capable of binding to targeted gene transcripts (mRNAs) that have partial complementarity to the sequence of microRNA. Mature microRNA is usually sized about 17-27 oligonucleotides in length and is able to either directly degrade its intracellular mRNA target(s) or suppress the protein translation of its targeted mRNA(s), depending on the complementarity between the microRNA and its target mRNA(s). Native microRNAs are found in almost all eukaryotes, functioning as a defense against viral infections and allowing regulation of specific gene expression during development of plants and animals. In principle, one microRNA often targeted multiple target mRNAs to fulfill its full functionality while on the other hand multiple miRNAs may target the same gene transcripts to enhance the effect of gene silencing.

The terms “MicroRNA Precursor” or “pre-miRNA” means hairpin-like single-stranded RNA containing stem-arm and stem-loop regions for interacting with intracellular RNase III Dicer endoribonucleases to produce one or multiple mature microRNAs (miRNAs) capable of silencing a targeted gene or a specific group of targeted genes that contain full or partial complementarity to the mature microRNA sequence(s). The stem-arm of a pre-miRNA can form either a perfectly (100%) or a partially (mis-matched) hybrid duplexes, while the stem-loop connects one end of the stem-arm duplex to form a circle or hairpin-loop conformation required for being assembled into the RNA-induced silencing complex (RISC) with some argonaute proteins (AGO).

As used herein, the term “neurologic disease or condition” intends a pathological condition having symptoms and clinical markers of fibrotic tissue, e.g., systemic sclerosis, renal, pulmonary, or cardiac hypertension, myocardial infarction, and chronic liver disease (e.g., hepatitis, alcoholic liver disease, or non-alcoholic steatohepatitis) and/or in various organs or tissues, e.g., liver, heart, kidney, lung, pancreas, the joints and the eye. Non-limiting examples of fibrotic conditions and associated disorders are provided in FIG. 9 and include without limitation, scleroderma, keloids and rheumatoid arthritis.

The terms “Neurons” or “neural cells” or “neural cell types” or “neural lineage” may include any neuron lineage cells, and can be taken to refer to cells at any stage of neuronal ontogeny without any restriction, unless otherwise specified. For example, neurons may include both neuron precursor cells, mature neurons and neural cell types such as astrocytes.

As used herein, the term “neural lineage cell” refers to a cell that contributes to the nervous system (both central and peripheral) or neural crest cell fates during development or in the adult. The nervous system includes the brain, spinal cord, and peripheral nervous system. Neural crest cell fates include cranial, trunk, vagal, sacral, and cardiac, giving rise to mesectoderm, cranial cartilage, cranial bone, thymus, teeth, melanocytes, iris pigment cells, cranial ganglia, dorsal root ganglia, sympathetic/parasympathetic ganglia, endocrine cells, enteric nervous system, and portions of the heart.

The terms “Non-coding RNA” or “ncRNA” means an RNA transcript that cannot be used to synthesize peptides or proteins through intracellular translation machineries. Non-coding RNA includes long and short regulatory RNA molecules such as microRNA (miRNA), small hairpin RNA (shRNA), small interfering RNA (siRNA) and double strand RNA (dsRNA). These regulatory RNA molecules usually function as gene silencers, interfering with expression of intracellular genes containing either completely or partially complementarity to the non-coding RNAs.

The term “Nucleic Acid Composition” refers to an oligonucleotide or polynucleotide such as a DNA or RNA sequence, or a mixed DNA/RNA sequence, in either a single-stranded or a double-stranded molecular structure.

The term “Nucleic Acid Template” means a double-stranded DNA molecule, double stranded RNA molecule, hybrid molecules such as DNA-RNA or RNA-DNA hybrid, or single-stranded DNA or RNA molecule.

The term “Nucleotide” means a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. A nucleoside containing at least one phosphate group bonded to the 3′ or 5′ position of the pentose is a nucleotide. DNA and RNA are consisted of different types of nucleotide units called deoxyribonucleotide and ribonucleotide, respectively

The term “Nucleotide Analog” means a purine or pyrimidine nucleotide that differs structurally from adenine (A), thymine (T), guanine (G), cytosine (C), or uracil (U), but is sufficiently similar to substitute for the normal nucleotide in a nucleic acid molecule.

The term “Oligonucleotide” means a molecule comprised of two or more monomeric units of DNA and/or RNA, preferably more than three, and usually more than ten. An oligonucleotide longer than 13 nucleotide monomers is also called polynucleotide. The exact size will depend on many factors, which in turn depends on the ultimate function or use of the oligonucleotide. The oligonucleotide may be generated in any manner, including chemical synthesis, DNA replication, RNA transcription, reverse transcription, or a combination thereof.

The terms “Overexpression” or “under expression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

The term “parkinsonism” refers to a group of diseases that are all linked to an insufficiency of dopamine in the basal ganglia which is a part of the brain that controls movement. Symptoms include tremor, bradykinesia (extreme slowness of movement), flexed posture, postural instability, and rigidity. A diagnosis of parkinsonism requires the presence of at least two of these symptoms, one of which must be tremor or bradykinesia. The most common form of parkinsonism is idiopathic, or classic, Parkinson's disease (PD), but for a significant minority of diagnoses, about 15 percent of the total, one of the Parkinson's plus syndromes (PPS) may be present. These syndromes also known as atypical parkinsonism, include cortico-basal degeneration, Lewy body dementia, multiple system atrophy, and progressive supranuclear palsy. In general, Parkinson's disease involves the malfunction and death of vital nerve cells in the brain primarily in an area of the brain called the substantia nigra. Many of these vital nerve cells make dopamine, that as these neurons die off, the amount of dopamine resulting from differentiation in the brain decreases, leaving a person unable to control movement normally. The intestines also have dopamine cells that degenerate in Parkinson's disease patients, and this may be an important causative factor in the gastrointestinal symptoms that are part of the disease. A group of symptoms that an individual experiences varies from person to person. Primary motor signs of Parkinson's disease include the following: tremor of the hands, arms, legs, jaw and face, bradykinesia or slowness of movement, rigidity or stiffness of the limbs and trunk and postural instability or impaired balance and coordination.

The term a “pharmaceutical composition” is intended to include the combination of an active exosome or population of cell populations with a carrier, inert or active such as a solid support, making the composition suitable for diagnostic or therapeutic use in vitro, in vivo or ex vivo.

The term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The terms “Pluripotency” or “pluripotent” refers to a stem cell or undifferentiated cell that has the potential to differentiate into all cells constituting one or more tissues or organs, for example, any of the three germ layers: endoderm (interior stomach lining, gastrointestinal tract, the lungs), mesoderm (muscle, bone, blood, urogenital), or ectoderm (epidermal tissues and nervous system).

The term “Posttranscriptional Gene Silencing” means a targeted gene knockout or knockdown effect at the level of mRNA degradation or translational suppression, which is usually triggered by either foreign/viral DNA or RNA transgenes or small inhibitory RNAs.

The terms “protein,” “polypeptide” and “peptide” are used interchangeably herein when referring to a gene product.

In one aspect of the disclosure, a “predetermined threshold level” or “threshold value” is used to categorize expression as high or low. As a non-limiting example of the disclosure, the threshold level of the miR of the exosome is a level of miR expression found in subjects that have been diagnosed with a fibrotic or hepatic disease or an associate disorder. Alternatively or in addition, the predetermined threshold level is the measured miRNA expression level for that individual subject prior to a subsequent measurement, e.g., prior to therapy or prior to an additional dose of the therapy.

In one aspect of the disclosure, miR expression can be provided as a ratio above the threshold level and therefore can be categorized as high expression or up-regulated, whereas a ratio below the threshold level is categorized as down-regulated or low expression.

The term “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present disclosure, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

The term “Primary RNA Transcript” means an RNA sequence that is directly transcribed from a gene without any RNA processing or modification, which may be selected from the group consisting of mRNA, hnRNA, rRNA, tRNA, snoRNA, snRNA, pre-microRNA, viral RNA and their RNA precursors as well as derivatives.

The terms “Precursor messenger RNA” or “pre-mRNA” means primary RNA transcripts of a protein-coding gene, which are produced by eukaryotic type-II RNA polymerase (Pol-II) machineries in eukaryotes through an intracellular mechanism termed transcription. A pre-mRNA sequence contains a 5′-untranslated region (UTR), a 3′-UTR, exons and introns.

The term “promoter” is used herein in its ordinary sense to refer to a nucleotide region comprising a DNA or RNA regulatory sequence, wherein the regulatory sequence is derived from a gene which is capable of binding RNA polymerase and initiating transcription of a downstream (3′ direction) coding sequence.

The terms “purified,” “to purify,” “purification,” “isolated,” “to isolate,” “isolation,” and grammatical equivalents thereof as used herein, refer to the reduction in the amount of at least one contaminant from a sample. For example, a desired cell type is purified by at least a 10%, preferably by at least 30%, more preferably by at least 50%, yet more preferably by at least 75%, and most preferably by at least 90%, with a corresponding reduction in the amount of undesirable cell types, for example, directed differentiation of the pluripotent cells resulted in the desired increase in purity of differentiated floor plate midbrain progenitor cells or midbrain fate FOXA2/LMX1A+ dopamine (DA) neurons. In other words “purify” and its equivalents, refers to the removal of certain cells (e.g., undesirable cells) from a sample either mechanically, such as by flow cytometer cell sorting or through directed differentiation. For example, for differentiating a purified population of forkhead box protein A2 (FOXA2)+ LIM homeobox transcription factor 1, alpha (LMX1A)+ progenitor cells, progenitor cells are purified by removal of contaminating PAX6 neuronal cells by sorting a mixed cell population into double positive forkhead box protein A2 (FOXA2)+ LIM homeobox transcription factor 1, alpha (LMX1A)+ cells by flow cytometry; midbrain fate FOXA2/LMX1A+ dopamine (DA) neurons are also purified or “selected” from non-dopamine (DA) (default cells) by using a specified method of cell culture comprising compositions and methods disclosed herein. The removal or selection of non-midbrain fate FOXA2/LMX1A+ dopamine (DA) neuronal cells results in an increase in the percent of desired midbrain fate FOXA2/LMX1A+ dopamine (DA) neurons in the sample. Thus, purification of a cell type results in an “enrichment,” i.e., an increase in the amount, of the desired cell, i.e. midbrain fate FOXA2/LMX1A+ dopamine (DA) neurons in the sample.

The terms “RNA Interference” or “RNAi” mean a posttranscriptional gene silencing mechanism in eukaryotes, which can be triggered by small inhibitory RNA molecules such as microRNA (miRNA), small hairpin RNA (shRNA) and small interfering RNA (siRNA). These small RNA molecules usually function as gene silencers, interfering with expression of intracellular genes containing either completely or partially complementarity to the small RNAs.

The term “RNA Processing” means a cellular mechanism responsible for RNA maturation, modification and degradation, including RNA splicing, intron excision, exosome digestion, nonsense-mediated decay (NMD), RNA editing, RNA processing, and a combination thereof.

As used herein, the term “SB431542” refers to a molecule capable of lowering or blocking transforming growth factor beta (TGFβ)/Activin-Nodal signaling with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide.

The term “Sense” means a nucleic acid molecule in the same sequence order and composition as the homologous mRNA. The sense conformation is indicated with a “+”, “s” or “sense” symbol.

As used herein, the term “Sma Mothers Against Decapentaplegic” or “Small Mothers Against Decapentaplegic” or “SMAD” refers to a signaling molecule.

The terms “Small or short hairpin RNA” or “shRNA” mean single-stranded RNA that contains a pair of partially or completely matched stem-arm nucleotide sequences divided by an unmatched loop oligonucleotide to form a hairpin-like structure. Many natural miRNAs are preserved as a form of shRNA in cells, such as precursor microRNA (pre-miRNA).

The terms “Small interfering RNA” or “siRNA” mean short double-stranded RNA sized about 18-27 perfectly base-paired ribonucleotide duplexes and capable of degrading target gene transcripts with almost perfect complementarity.

As used herein, the teen “Sonic hedgehog (SHH or Shh” refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). Shh typically binds to PCT which then allows the activation of SMO as a signal transducer. In the absence of SHH, PTC typically inhibits SMO, which in turn activates a transcriptional repressor so transcription of certain genes does not occur. When Shh is present and binds to PTC, PTC cannot interfere with the functioning of SMO. With SMO uninhibited, certain proteins are able to enter the nucleus and act as transcription factors allowing certain genes to be activated (see, Gilbert, 2000 Developmental Biology (Sunderland, Mass.: Sinauer Associates, Inc., Publishers).

As used herein, the term “stem cell” refers to a cell with the ability to divide for indefinite periods in culture and to give rise to specialized cells. A stem cell may be obtained from animals and patients, including humans; for example, a human stem cell refers to a stem cell that is human. A stem cell may be obtained from a variety of sources including embryonic and nonembryonic, such as umbilical cord cells, cells from children and cells from adults. Adult stem cells in general refer to cells that were not originally obtained from a fetus, in other words, cells from babies, cast off umbilical cords, cast off placental cells, cells from children, cells from adults, etc.

The terms “subject,” “individual” or “patient” are used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Suspension culture,” refers to a culture in which cells, or aggregates of cells, multiply while suspended in liquid medium.

The terms “suitable for a therapy” or “suitably treated with a therapy” mean that the patient is likely to exhibit one or more desirable clinical outcome as compared to patients having the same disease and receiving the same therapy but possessing a different characteristic that is under consideration for the purpose of the comparison.

The term “Template” means a nucleic acid molecule being copied by a nucleic acid polymerase. A template can be single-stranded, double-stranded or partially double-stranded, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

The term “transgene,” refers to a gene, nucleic acid, or polynucleotide which has been introduced into the cell or organism by artificial or natural means, such as an exogenous nucleic acid. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature.

“Transfection” refers to a method of gene delivery that introduces a foreign nucleotide sequences (e.g. DNA or RNA molecules) into a cell preferably by a non-viral method. In some embodiments, foreign DNA or RNA is introduced to a cell by transient transfection of an expression vector encoding a polypeptide of interest, whereby the foreign DNA or RNA is introduced but eliminated over time by the cell and during mitosis. By “transient transfection” is meant a method where the introduced expression vectors and the polypeptide encoded by the vector, are not permanently integrated into the genome of the host cell, or anywhere in the cell, and therefore may be eliminated from the host cell or its progeny over time.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For example, in the case of Parkinson's disease, the term “treatment” intends a more favorable clinical assessment by a treating physician or assistant and/or reduced expression of fibrosis markers, e.g., αSMA, CTGF, collagen, matrix molecules and/or a shift toward normal read-outs in tests that diagnose liver function and/or Parkinson's disease.

As used herein, the term “undifferentiated” refers to a cell that has not yet developed into a specialized cell type.

The term “Vector” mans a recombinant nucleic acid composition such as recombinant DNA (rDNA) or RNA capable of movement and residence in different genetic environments. Generally, another nucleic acid is operatively linked therein. The vector can be capable of autonomous replication in a cell in which case the vector and the attached segment is replicated. One type of preferred vector is an episome, i.e., a nucleic acid molecule capable of extrachromosomal replication. Preferred vectors are those capable of autonomous replication and expression of nucleic acids. Vectors capable of directing the expression of genes encoding for one or more polypeptides and/or non-coding RNAs are referred to herein as “expression vectors” or “expression-competent vectors”. Particularly important vectors allow cloning of cDNA from mRNAs produced using a reverse transcriptase. A vector may contain components consisting of a viral or a type-II RNA polymerase (Pol-II or pol-2) promoter, or both, a Kozak consensus translation initiation site, polyadenylation signals, a plurality of restriction/cloning sites, a pUC origin of replication, a SV40 early promoter for expressing at least an antibiotic resistance gene in replication-competent prokaryotic cells, an optional SV40 origin for replication in mammalian cells, and/or a tetracycline responsive element. The structure of a vector can be a linear or circular form of single- or double-stranded DNA or RNA selected form the group consisting of plasmid, viral vector, transposon, retrotransposon, DNA or RNA transgene, jumping gene, and a combination thereof.

As used herein, the term “WNT” or “wingless” in reference to a ligand refers to a group of secreted proteins (i.e. Int1 (integration 1) in humans) capable of interacting with a WNT receptor, such as a receptor in the Frizzled and LRPDerailed/RYK receptor family.

The term “5′-end” means a terminus lacking a nucleotide at the 5′ position of successive nucleotides in which the 5′-hydroxyl group of one nucleotide is joined to the 3′-hydroyl group of the next nucleotide by a phosphodiester linkage. Other groups, such as one or more phosphates, may be present on the terminus.

The term “3′-end” means a terminus lacking a nucleotide at the 3′ position of successive nucleotides in which the 5′-hydroxyl group of one nucleotide is joined to the 3′-hydroyl group of the next nucleotide by a phosphodiester linkage. Other groups, most often a hydroxyl group, may be present on the terminus.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is noted that for each miR described herein the corresponding sequence (mature and precursor) is provided in the sequence listing which should be regarded as part of the specification.

Unless otherwise defined, all technical and/or scientific terms used herein have the same meaning as commonly understood by one of ordinary skill in the art. Although methods and materials similar or equivalent to those described herein can be used in the practice or testing of embodiments disclosed herein, exemplary methods and/or materials are described below. In case of conflict, the patent specification, including definitions, will control. In addition, the materials, methods, and examples are illustrative only and are not intended to be necessarily limiting.

Before explaining at least one embodiment in detail, it is to be understood that the disclosure is not necessarily limited in its application to the details set forth in the following description or exemplified by the Examples. The disclosure is capable of other embodiments or of being practiced or carried out in various ways.

### Compositions

### Sequence IDs

SEQ ID No 1: >human hsa-miR-155-5p MIMAT0000646
UUAAUGCUAAUCGUGAUAGGGGUU
SEQ ID No 2: >mouse miR-155-5p MIMAT0000646
UUAAUGCUAAUUGUGAUAGGGGUU
SEQ ID No 3: >human hsa-miR-155-3p MIMAT0004658
CUCCUACAUAUUAGCAUUAACA
SEQ ID No 4: >mouse -miR-155-3p MIMAT0004658
CUCCUACCUGUUAGCAUUAACA
SEQ ID No 5: >hsa-miR-155 stem loop MI0000681
CUGUUAAUGCUAAUCGUGAUAGGGGUUUUUGCCUCCAACUGACUC
CUACAUAUUAGCAUUAACAG
SEQ ID No 6: >modified hsa-miR-155-5p
GGAAUGCUAAUCGUGAUAGGGGUU
SEQ ID No 7: >modified hsa-miR-155-3p
UUCCUACAUAUUAGCAUUAACA
SEQ ID No 8: >modified hsa-miR-155 stem loop
CUGUUAAUGCUAAUCGUGAUAGGGAUUUUUGCCUCCAACUGAUU
CCUACAUAUUAGCAUUAACAG
SEQ ID No 9: >miR-155-3p isomiR
3′-ACC**UACGAUUAUACAUCCUC
SEQ ID No 10: >miR-155-3p isomiR
3′-*CAAUUACGAUUAUACAUCCU*
SEQ ID No 11: >miR-155-3p isomiR
3′-ACAAUUACGAUUAUACAUCCU*
SEQ ID No 12: >miR-155-3p isomiR
3′-***ACUACGAUUAUACAUCCUC
SEQ ID No 13: >miR-155-3p isomiR
3′-**AAUUACGAUUAUACAUCCU*
SEQ ID No 14: >miR-155-3p isomiR
3′-***AUUACGAUUAUACAUCCUC
SEQ ID No 15: >miR-155-3p isomiR
3′-******ACGAUUAUACAUCCUCA
SEQ ID No 16: >miR-155-3p isomiR
3′-******ACGAUUAUACAUCCUCAG
SEQ ID No 17: >miR-155-3p isomiR
3′-*CAA*UACGAUUAUACAUCCUC
SEQ ID No 18: >miR-155-3p isomiR
3′-*** AUUACGAUUAUACAUCCUCA
SEQ ID No 19: >miR-155-3p isomiR
3′-*** CUUACGAUUAUACAUCCUC
SEQ ID No 20: >miR-155-3p isomiR
3′-ACAAUUACGAUUAUACAU****
SEQ ID No 21: >miR-155-3p isomiR
3′-****UUACGAUUAUACAUCCUC

### Pre-miR-155

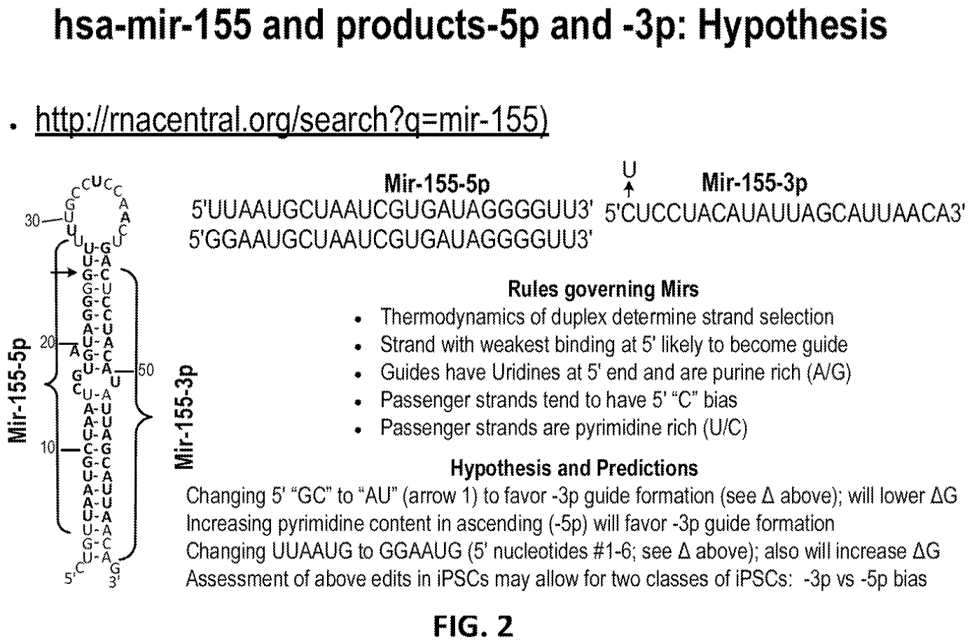

MIR-155 (MicroRNA 155) is an RNA Gene, and is affiliated with the miRNA class. Diseases associated with MIR-155 include Diffuse Large B-Cell Lymphoma and Pancreatic Ductal Adenocarcinoma. Among its related pathways are Cell differentiation-expanded index and Toll-like receptor signaling pathway. FIG. 2 shows the sequence of the pre-miR-155 stem loop that is matured from the pri-miRNA transcript. The mature miR-155 (miR-155-5p) sequence is on the left and mature miR-155* (miR-155-3p) sequence is shown on the right.

MiR-155 is processed from an exon of a noncoding RNA transcribed from the B-cell integration cluster (BIC) located on chromosome 21

miRNA-155 sequence is 24 nucleotides long and is found in Homo sapiens. Homo sapiens (human) hsa-miR-155-5p sequence is a product of miR-155-5p, hsa-miR-155, MIR-155, miR-155, hsa-miR-155-5p genes.

MicroRNA-155 (miR-155) exists as two mature isoforms-miR-155-3p and miR-155-5p, generated by ribonuclease processing of the pre-miR-155 stem loop. Stated another way the miR-155-3p/5p products are a result of loading into the RISC complex where one strand is preferentially loaded (as described in the intro).

In certain aspects, disclosed is a composition comprising, consisting essentially of, or consisting of one or more modified pre-miR-155. In some embodiments, the miRNA has complete complementarity with the wildtype miRNA sequence except for 1, 2, or 3 nucleotide substitutions, terminal additions, and/or truncations.

Disclosed is a modified pre-miR-155 having SEQ ID No: 6. Disclosed is a modified pre-miR-155 having SEQ ID No: 7. Disclosed is a modified pre-miR-155 stem loop having SEQ ID No: 8. Disclosed is a modified pre-miR-155 having SEQ ID No: 6 and SEQ ID No: 7. Disclosed is a modified pre-miR-155 having SEQ ID No: 6, and SEQ ID No: 7, and a pre-miR-155 stem loop having SEQ ID No: 8. Disclosed is a modified pre-miR-155 having SEQ ID No: 6 and a pre-miR-155 stem loop having SEQ ID No: 8. Disclosed is a modified pre-miR-155 having SEQ ID No: 7 and a pre-miR-155 stem loop having SEQ ID No: 8.

In some embodiments, the miRNA-155 is a pre-miRNA. In some embodiments, the miRNA-155 has Seq ID No 1. In some embodiments, the miRNA-155 has Seq ID No 2. In some embodiments, the miRNA-155 has Seq ID No 3. In some embodiments, the miRNA-155 has Seq ID No 4. In some embodiments, the miRNA-155 has Seq ID No 5. In some embodiments, the miRNA-155 has Seq ID No 6. In some embodiments, the miRNA-155 has Seq ID No 7. In some embodiments, the miRNA-155 stem loop has Seq ID No 8. In some embodiments, the miRNA-155 has Seq ID No 6 and SEQ ID No 7. In some embodiments, the miRNA-155 has Seq ID No 6, SEQ ID No 7 and the miRNA-155 stem loop has SEQ ID No 8. In some embodiments, the miRNA-155 has Seq ID No 6 and the miRNA-155 stem loop has SEQ ID No 8. In some embodiments, the miRNA-155 has SEQ ID No 7 and the miRNA-155 stem loop has SEQ ID No 8. In some embodiments, the miRNA-155 has SEQ ID No 9. In some embodiments, the miRNA-155 has SEQ ID No 10. In some embodiments, the miRNA-155 has SEQ ID No 11. In some embodiments, the miRNA-155 has SEQ ID No 12. In some embodiments, the miRNA-155 has SEQ ID No 13. In some embodiments, the miRNA-155 has SEQ ID No 14. In some embodiments, the miRNA-155 has SEQ ID No 15. In some embodiments, the miRNA-155 has SEQ ID No 16. In some embodiments, the miRNA-155 has SEQ ID No 17. In some embodiments, the miRNA-155 has SEQ ID No 18. In some embodiments, the miRNA-155 has SEQ ID No 19. In some embodiments, the miRNA-155 has SEQ ID No 20. In some embodiments, the miRNA-155 has SEQ ID No 21.

Disclosed is a pluripotent cell having a modified pre-miR-155, which favors miR-155-5p guide formation or strand selection in DA neural cells. Disclosed is a pluripotent cell having a modified pre-miR-155, which favors miR-155-3p guide formation or strand selection in DA neural cells. Disclosed is a pluripotent cell having a modified pre-miR-155, which decreases miR-155-5p guide formation or strand selection in DA neural cells. Disclosed is a pluripotent cell having a modified pre-miR-155, which decreases miR-155-3p guide formation or strand selection in DA neural cells.

In some embodiments, disclosed is a pluripotent cell having a modified pre-miR-155 comprises SEQ ID NO 6, which favors miR-155-3p strand selection in DA neural cells. In some embodiments, disclosed is a pluripotent cell having a modified pre-miR-155 comprises SEQ ID NO 7, which favors miR-155-3p strand selection. In some embodiments, the modified pluripotent cell having a pre-miR-155 stem loop comprises SEQ ID NO 8, which favors miR-155-3p strand selection in DA neural cells. In some embodiments, the pluripotent cell having a modified pre-miR-155 comprises SEQ ID NO 6 and SEQ ID NO 7, which favors miR-155-3p strand selection. In some embodiments, disclosed is a pluripotent cell having a modified pre-miR-155 comprises SEQ ID NO 6 and SEQ ID NO 7 and the modified pre-miR-155 stem loop comprises SEQ ID NO 8, which favors miR-155-3p strand selection in DA neural cells. In some embodiments, disclosed is a pluripotent cell having a modified pre-miR-155 comprises SEQ ID NO 6 and the modified pre-miR-155 stem loop comprises SEQ ID NO 8, which favors miR-155-3p strand selection in DA neural cells. In some embodiments, disclosed is a pluripotent cell having a modified pre-miR-155 comprises SEQ ID NO 7 and the modified pre-miR-155 stem loop comprises SEQ ID NO 8, which favors miR-155-3p strand selection in DA neural cells.

In some embodiments, disclosed is a pluripotent cell having a modified pre-miR-155 comprises increasing pyrimidine content to favor miR-155-3p strand selection in DA neural cells. In some embodiments, disclosed is a pluripotent cell having a modified pre-miR-155 comprises increasing pyrimidine content to decreases miR-155-3p strand selection in DA neural cells.

In certain aspects, disclosed is a pre-miRNA-155 sequence, wherein the pre-miRNA-155 incorporated into a DA neuronal cell has an anti-inflammatory effect on human subjects. In certain aspects, disclosed is a pre-miRNA-155 sequence, wherein the pre-miRNA-155 incorporated into a DA neuronal cell has a pro-inflammatory effect on human subjects.

### MicroRNA

### Methods

Disclosed are methods of ex vivo differentiation of pluripotent cells towards neural progenitor cells and DA neurons using modified microRNAs. In some embodiments, the cell population is differentiated using a mono-SMAD, or dual-SMAD method disclosed herein. In some embodiments, the cell population is differentiated using a mono-SMAD, or dual-SMAD method disclosed herein or another approach. In some embodiments, the miR-155-5p is modified. In some embodiments, the miR-155-3p is modified. In some embodiments, the miR-155-5p stem loop is modified.

Modified miRNA, modified miR-155, modified miR-155-5p, modified miR-155-3p, modified miR-155-stem loop can be prepared by any appropriate method, e.g., by isolation form natural products such as cell populations or recombinantly produced, for example, by a chemical synthetic method or a method using genetic recombination technique. When the production is carried out by a method using genetic recombination technique, miRNA can, for example, be produced through a transcription reaction with use of a DNA template and a RNA polymerase obtained by means of gene recombination. Examples of suitable RNA polymerase include a T7 RNA polymerase, a T3 RNA polymerase, and a SP6 RNA polymerase. They can be produced in a eukaryotic or prokaryotic cells, e.g., E. coli or other bacteria, yeast, mammalian, human, murine or simian for example.

In some instances, the miRNAs or nucleic acids encoding the miRNA are produced synthetically using well-known methods or are isolated from cells or tissues. Typically, the miRNAs or nucleic acid molecules containing or encoding the miRNAs are obtained using genetic engineering techniques to produce a recombinant nucleic acid molecule, which can then be isolated or purified by techniques well known to one of ordinary skill in the art. In these recombinant methods, nucleic acid encoding the miRNA is cloned into an appropriate expression vector. It is well within the skill of a skilled artisan to design DNA that encodes a miRNA provided herein.

Any suitable host/vector system can be used to express one or more of the miRNAs described herein. It is well with the skill of those in the art to select an appropriate system based on, for example, whether the miRNA or nucleic acid molecule encoding the miRNA is being isolated and purified for subsequent use, and/or whether the miRNA will be expressed in vivo following administration to a subject.

In particular examples, the miRNAs described herein (including precursor miRNAs) are encoded by vectors for expression of the miRNA in vivo following administration of the vector to a subject. The choice of vector, including the particular regulatory elements contained in the vector for expression of heterologous nucleic acid, can be influenced by the cell type to which the vector is being targeted, and such selection is well within the level of skill of the skilled artisan. For example, the nucleic acid encoding the miRNA can be under the control of a tissue- or cell-specific promoter, such that the miRNA is only expressed in that particular tissue or cell type. Tissue- or cell-specific promoters are well known in the art.

In further examples, the nucleic acid encoding the miRNA is cloned into a viral vector, including, but not limited to, retroviral, adenoviral, lentiviral and adeno-associated viral vectors. Although viral vectors can be replication incompetent or replication competent, for subsequent use in therapeutic applications, typically replication incompetent vectors are selected.

The activity of the miRNAs can be assessed using in vitro assays and animal models well known to those skilled in the art. The miRNAs also can be assessed in human clinical trials under appropriate supervision.

Disclosed is a method for affecting miRNA strand selection. Disclosed is a method for affecting pre-miR-155 strand selection. Disclosed is a method for affecting miR-155-3p strand selection. Disclosed is a method for affecting miR-155-5p strand selection. In some embodiments, the strand selection is in pluripotent cells.

Disclosed is a method for affecting miR-155-3p and miR-155-5p strand ratio in pluripotent cells. Disclosed is a method for affecting miR-155-3p and miR-155-5p strand ratio in pluripotent cells. In some embodiments, the ratio of miR-155-3p to miR-155-5p is greater. In some embodiments, the ratio of miR-155-5p to miR-155-3p is greater. In some embodiments, miR-155-3p is 5% greater than miR-155-3p. In some embodiments, miR-155-3p is 5%, 6%, 7%, 8%, 9%, 10%, 20%, 25%, 30%, 40%, 50%, 60%, 70%, 80%, 90%, 95% or 99% greater than miR-155-5p. In some embodiments, miR-155-5p is 5%, 6%, 7%, 8%, 9%, 10%, 20%, 25%, 30%, 40%, 50%, 60%, 70%, 80%, 90%, 95% or 99% greater than miR-155-3p.

Whether affecting miRNA strand selection or miR-155-3p and miR-155-5p strand ratio in pluripotent cells, in some embodiments, the method comprises modifying miR-155-3p. In some embodiments, the method comprises modifying miR-155-5p. In some embodiments, the method comprises modifying pre-miR-155-stem loop. In some embodiments, the method comprises modifying the miR-155-3p, and the modifying miR-155-5p. In some embodiments, the method comprises modifying the miR-155-3p, modifying the miR-155-5p and modifying the pre-miR-155-stem loop. In some embodiments, the method comprises modifying the miR-155-3p and modifying the pre-miR-155-stem loop. In some embodiments, the method comprises modifying the miR-155-5p and modifying the pre-miR-155-stem loop. In some embodiments, the modification comprises SEQ ID No 6, SEQ ID No 7, or SEQ ID No 8. In some embodiments, the modification comprises SEQ ID Nos 9-21.

Gene Editing iPSC

### Methods

In some embodiments, the method comprises gene editing the miR-155-3p in a pluripotent cell or pluripotent cell population. In some embodiments, the method comprises gene editing the miR-155-5p in a pluripotent cell or pluripotent cell population. In some embodiments, the method comprises gene editing the pre-miR-155-stem loop in a pluripotent cell or pluripotent cell population. In some embodiments, the gene editing comprises changing the miR-155-3p 5′ nucleotides 1-2 in a pluripotent cell or pluripotent cell population. In some embodiments, the gene editing comprises changing the miR-155-3p 5′ nucleotide 1 in a pluripotent cell or pluripotent cell population. In some embodiments, the gene editing comprises changing the miR-155-3p 5′ nucleotide 1 from a C to a U in a pluripotent cell or pluripotent cell population. In some embodiments, the gene editing comprises changing miR-155-5p 5′ nucleotides 1-2 in a pluripotent cell or pluripotent cell population. In some embodiments, the gene editing comprises changing miR-155-5p 5′ nucleotides 1-6 in a pluripotent cell or pluripotent cell population. In some embodiments, the gene editing comprises changing miR-155-5p 5′ nucleotides 1-6 from UUAAUG to GGAAUG in a pluripotent cell or pluripotent cell population. In some embodiments, the gene editing comprises changing one nucleotide base pair in the stem loop in a pluripotent cell or pluripotent cell population. In some embodiments, the gene editing comprises changing nucleotide 25 from a G to an A and changing nucleotide 43 from a C to a U in the stem loop in a pluripotent cell or pluripotent cell population.

Disclosed is a method for affecting miRNA-155miR-155-3p and miR-155-3p strand ratio in DA neural cells. In some embodiments, the method comprises affecting miRNA strand selection in pluripotent cells as discussed above and differentiating the modified miRNA pluripotent cells to DA neural cells. In some embodiments, the method comprises modifying miR-155-3p and/or miR-155-5p and/or pre-miR-155-stem loop in pluripotent cells as discussed above and differentiating the modified miRNA pluripotent cells to DA neural cells.

In some embodiments, a method of gene editing pluripotent stem cells is disclosed.

### Gene Editing Techniques

### Transiently Transfecting the Pluripotent Cells

Transfection is the introduction of DNA, RNA, or proteins into eukaryotic cells and is used in research to study and modulate gene expression. Thus, transfection techniques serve as an analytical tool that facilitates the characterization of genetic functions, protein synthesis, cell growth and development. Transfection assays not only enable the advancement of cellular research, but also enhance drug discovery strategies. Similar strategies, such as viral transfection or viral transduction, utilize lentiviral particles to insert foreign material into eukaryotic cells. Whereas bacterial transformation is the process of horizontal gene transfer where bacteria uptake foreign genetic material.

There is a wide range of transfection methods that include physical, chemical and biological techniques. These techniques generally involve the use of transient or stable transfection methods to incorporate nucleic acids into cells.

Transient transfection techniques involve the introduction of DNA or RNA into cells, but in this method, the DNA or RNA does not integrate with the cellular chromosomes. This technique facilitates high transfection efficiencies and the gene transcripts can be analyzed after a period of 1-4 days. For large-scale transient gene expression (TGE) in mammalian cell cultures, transfection vehicles such as polyethylenimine (PEI) and calcium phosphate (CaPi) can be used. Furthermore, large-scale TGE methods have also been developed using Chinese hamster ovary (CHO) cells in the absence of serum¹.

Stable transfection techniques involve the integration of the transfected DNA or RNA into cellular chromosomes or the formation of episomes. The stably transfected cell can be subsequently identified using selectable markers such as dihydrofolate reductase (DHFR), hygromycin B phosphotransferase (HPH) and adenosine deaminase (ADA) among several others.

Some of the commonly used transfection techniques include calcium phosphate precipitation, lipofection, electroporation, and viral delivery. Additionally, these methods can be used in co-transfections. These techniques involve the simultaneous delivery of two distinct nucleic acids into the same cell and are often used to achieve stable transfections. Transfections methods have evolved to include several new methods such as the biolistic delivery systems that use high velocity microparticles to deliver nucleic acids into cells, and in vivo transfection protocols that facilitate systemic delivery of siRNA molecules.

### Calcium Phosphate Transfection

The calcium phosphate transfection technique involves the precipitation of DNA or RNA and calcium phosphate. The precipitation is facilitated by mixing a HEPES-buffered saline solution, having sodium phosphate, with calcium chloride solution and DNA or RNA. Glycerol shock is often used to enhance the DNA OR RNA uptake in certain cells.

While this technique is cost-efficient and can be used for transient or stable transfections in a wide range of cells, relatively small changes in pH (±0.1) can affect the efficiency of transformation. Furthermore, it is essential to maintain reagent consistency for reproducing the assay results. However, this transfection method does not work in RPMI, or other media with high phosphate concentrations.

### Liposome-Mediated Transfection

Liposome-mediated transfection (lipofection) techniques involve the use of liposome forming cationic lipids, or non-lipid polymers. Examples of lipofection transfection reagents may include DOTMA (N-[1-(2,3,-dioleyloxy)propyl]-N,N,N-trimethylammonium chloride) and X-tremeGENE™ transfection reagents, suitable for transfecting a variety of DNA or RNA, small RNA, CRISPR/Cas7-11 and CRISPR/Cas13 components into a diverse range of cell lines. Lipid transfections can also be adapted for cost-effective, as well as high-throughput systems; however, these transfections are usually cell-type specific.

### Electroporation

This technique involves the exposure of cell membranes to high-intensity electric pulses which causes a temporary destabilization in certain areas of the cell. During this transient destabilization event, the cell membrane becomes highly permeable and allows the entry of various exogenous molecules including DNA or RNA.

Electroporation is an easy, non-chemical technique that can yield high transformation efficiencies in various cell types. Although this technique does not alter target cell morphology and functions, the method can cause cell death if transfection is not performed under optimum conditions.

### Viral Transfection (Viral Transduction)

This method involves the use of viral vectors to deliver nucleic acids into cells. Viral delivery systems such as lentiviral, adenoviral and oncoretroviral vectors can be used for transferring nucleic acids, even in hard-to-transfect cells.

Although viral delivery methods are highly efficient, they can be quite laborious. Moreover, most viruses require containment and careful monitoring of biosafety levels. Before performing viral transfections, it is also important to consider several limiting factors such as the lytic nature of viral vectors, cell line packaging and host-cell specificity.

In some embodiments, modifying pluripotent cells may be affected in a number of ways:
8. Transiently transfecting the pluripotent cells with the mature miRNA (or modified form thereof, as described herein below). The miRNAs designed according to the teachings herein can be generated according to any oligonucleotide synthesis method known in the art, including both enzymatic syntheses and solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. 5 (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.
9. Stably, or transiently transfecting the pluripotent cells with an expression vector which encodes the mature miRNA or with miRNA mimic.
10. Stably, or transiently transfecting the pluripotent cells with an expression vector which encodes the pre-miRNA. The pre-miRNA sequence may comprise from 1-17, 1-50, 19-25, 45-90, 60-80 or 60-70 nucleotides. The sequence of the pre-miRNA may comprise a miR-155-5p and/or a miR-155-3p as set forth herein. The sequence of the pre-miRNA may also be that of a modified pri-miRNA such as SEQ ID No 6-8. The sequence of the pre-miRNA may comprise the sequence of the miRNA, or variants thereof.
11. Stably, or transiently transfecting the pluripotent cells with an expression vector which encodes the pri-miRNA. The sequence of the pri-miRNA may comprise a pre-miRNA, miR-155-5p and/or miR-155-3p, as set forth herein, and variants thereof. Preparation of miRNAs mimics can be affected by chemical synthesis methods or by recombinant methods.

miRNA antagonists may be introduced into cells using transfection protocols known in the art using either siRNAs or expression vectors such as Anatgomirs.

Altering Strand Selection without Gene Editing

In another alternative, it is possible to add an agent to the pluripotent cells (either during incubation or maturation) which alters the strand selection (-3p or miR-155-3p) without altering the genotype of the cell (i.e. without performing gene editing). Mechanisms by which transcription may be so altered include activating, deactivating or mimicking signaling pathways in a cell, and up-regulating, down-regulating, activating or inactivating transcription factors (at the level of transcription, translation or post-translationally). Transcription factor activity may be modulated at the post-transcriptional level by e.g. stimulating their phosphorylation/dephosphorylation, or targeting them for degradation by modulating the ubiquitination activity of the cell. Such a compound may be a small molecule, such as a pharmaceutical, or a small biological molecule such as a peptide or a signaling molecule such as CAMP or cGMP. Such a compound may alternatively be a macromolecule such as a protein.

Disclosed are methods for differentiation of pluripotent cells towards miR-155-3p or miR-155-5p biased DA neurons by down-regulation of particular miRNAs-namely miR-155-3p or miR-155-5p. Disclosed are methods for differentiation of pluripotent cells towards miR-155-3p biased DA neurons and miR-155-5p un-biased DA neurons by down-regulation of particular miRNAs-namely miR-155-3p or miR-155-5p. Down-regulating such miRNAs can be affected using a polynucleotide which is hybridizable in cells under physiological conditions to the miRNA molecule or gene editing techniques. In some embodiments, the pluripotent cells are differentiated using a mono-SMAD, or dual-SMAD method disclosed herein to produce DA neurons.

Polynucleotides which Down-Regulate the miRNAs

As mentioned herein above, the polynucleotides which down-regulate the miRNAs described herein above may be provided as modified polynucleotides using various methods known in the art.

For example, the oligonucleotides (e.g. miRNAs) or polynucleotides may comprise heterocylic nucleosides consisting of purines and the pyrimidines bases, bonded in a 3′-to-5′ phosphodiester linkage. Preferably used oligonucleotides or polynucleotides are those modified either in backbone, inter-nucleoside linkages, or bases, as is broadly described herein under.

Specific examples of preferred oligonucleotides or polynucleotides useful according to this aspect include oligonucleotides or polynucleotides containing modified backbones or non-natural inter-nucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677, 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used are those modified in both sugar and the intemucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used are disclosed in U.S. Pat. No. 6,303,374.

Oligonucleotides or polynucleotides may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of oligomeric compounds. These include 5-substituted pyrimidines, 6-azapyrimidines, and N-2, N-6, and 0-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-0-methoxyethyl sugar modifications.

### Promoter Sequence for Directing Transcription

To express miRNAs or polynucleotide agents which regulate miRNAs in pluripotent cells or DA neural cells, a polynucleotide sequence encoding the miRNA (or pre-miRNA, or pri-miRNA, or polynucleotide which down-regulates the miRNAs) is preferably ligated into a nucleic acid construct suitable for pluripotent cell (or DA neural cell) expression. Such a nucleic acid construct includes a promoter sequence for directing transcription of the polynucleotide sequence in the cell in a constitutive or inducible manner.

It will be appreciated that the nucleic acid construct of some embodiments disclosed herein can also utilize miRNA homologues which exhibit the desired activity (e.g. DA cell differentiating ability). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any of the sequences described herein above, as determined using the BestFit software of the Wisconsin sequence analysis package, utilizing the Smith and Waterman algorithm, where gap weight equals 50, length weight equals 3, average match equals 10 and average mismatch equals −9.

In addition, the homologues can be, for example, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the sequences described herein above, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments disclosed herein are promoter sequences which are active under most environmental conditions and most types of cells such as the cytomegalovirus (CMV) and Rous sarcoma virus (RSV). Inducible promoters suitable for use with some embodiments disclosed herein include for example tetracycline-inducible promoter (Zabala M, et al., Cancer Res. 2004, 64 (8): 2799-804).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments disclosed herein is active in the specific cell population transformed—i.e. pluripotent cells.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

In addition to the elements already described, the expression vector of some embodiments may typically contain other specialized elements intended to increase the level of expression of cloned nucleic acids or to facilitate the identification of cells that carry the recombinant DNA or RNA. For example, a number of animal viruses contain DNA or RNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA or RNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives. Other expression vectors are available from SBI or Sigma.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMTO1O/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-1) and kidney cells may be targeted using the heterologous promoter present in the baculovirus Autographa californica nucleopolyhedrovirus (AcMNPV) as described in Liang C Y et al., 2004 (Arch Virol. 149:51-60).

According to one embodiment, a lentiviral vector is used to transfect the pluripotent cells.

Various methods can be used to introduce the expression vector of some embodiments into pluripotent cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. Biotechniques 4 (6): 504-512, 1986 and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation since higher transfection efficiency can be obtained due to the infectious nature of viruses.

Other vectors can be used that are non-viral, such as cationic lipids, polylysine, and dendrimers.

### Transfection Using Nanoparticles or Minicircle DNA/RNA Vectors

The miRNAs, miRNA mimics and pre-miR can be transfected into cells also using nanoparticles such as gold nanoparticles and by ferric oxide magnetic NP—see for example Ghosh et al., Biomaterials. 2013 January; 34 (3): 807-16; Crew E, et al., Anal Chem. 2012 Jan. 3; 84 (1): 26-9.

Other modes of transfection that do not involved integration include the use of minicircle DNA/RNA vectors or the use of PiggyBac transposon that allows the transfection of genes that can be later removed from the genome.

Host-Expression Systems to Express the miRNAs

As mentioned hereinabove, a variety of prokaryotic or eukaryotic cells can be used as host-expression systems to express the miRNAs or polynucleotide agent capable of down-regulating the miRNA of some embodiments. These include, but are not limited to, microorganisms, such as bacteria transformed with a recombinant bacteriophage DNA/RNA, plasmid DNA/RNA or cosmid DNA/RNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the miRNAs of some embodiments.

Examples of bacterial constructs include the pET series of E. coli expression vectors Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA/RNA sequences into the yeast chromosome.

The conditions used for contacting the pluripotent cells are selected for a time period/concentration of cells/concentration of miRNA/ratio between cells and miRNA which enable the miRNA (or inhibitors thereof) to induce differentiation thereof. The present disclosure further contemplates incubation of the stem cells with a differentiation factor which promotes differentiation towards a DA neural cell population. The incubation with such differentiation factors may be affected prior to, concomitant with or following the contacting with the miRNA. Examples of such agents are provided in the Examples section herein below.

### CRISPR/Cas13 Systems for Gene Editing

Alternatively, or additionally, the pluripotent cells may be genetically modified so as to express such differentiation factors, using expression constructs such as those described herein above. Further, the pluripotent cells can be genetically modified using the CRISPR/Cas13 system, CRISPR/Cas7-11 system or an equivalent system, to no longer express a target which is being silenced/down-regulated, such as for example pre-miR-155, miR-155-5p or miR-155-3p.

Several gene editing techniques are known in the art, but most preferably the CRISPR/Cas13 technique may be used to generate the modified pluripotent cells. This process is well known to those skilled in the art. The CRISPR/Cas13 technique employs the use of a single guide RNA (sgRNA) and a Cas13 nuclease.

### Certain Rules Govern Strand Selection of MirRNA:

Thermodynamics of duplex determine strand selection
Strand with weakest binding at 5′ likely to become guide
Guides have Uridines at 5′ end and are purine rich (A/G)
Passenger strands tend to have 5′ “C” bias
Passenger strands are pyrimidine rich (U/C)

Applicant discovered that changing 5′ “GC” to “AU” (arrow 1 in FIG. 2) in pluripotent cells will favor miR-155-3p strand selection in DA neural cells. Applicant discovered that changing 5′ “GC” to “AU” (arrow 1 in FIG. 2) in pluripotent cells will reduce wildtype miR-155-3p strand selection in DA neural cells. Thus, Applicant discovered that changing 5′ “GC” to “AU” (arrow 1 in FIG. 2) in pluripotent cells will favor modified miR-155-3p strand selection in DA neural cells and will reduce wildtype miR-155-3p strand selection in DA neural cells. This is shown in SEQ ID No 8 which is reproduced here with the modified bases underlined: SEQ ID No 8:

CUGUUAAUGCUAAUCGUGAUAGGGAUUUUUGCCUCCAACUGAUUCCUACAU
AUUAGCAUUAACAG

Applicant also discovered that changing 5′ “GC” to “AU” (arrow 1 in FIG. 2) in pluripotent cells will reduce wildtype miR-155-5p strand selection in DA neural cells. Thus, Applicant also discovered that changing 5′ “GC” to “AU” (arrow 1 in FIG. 2) in pluripotent cells will favor miR-155-3p strand selection in DA neural cells and will reduce wildtype miR-155-5p strand selection in DA neural cells. This is shown in SEQ ID No 8 which is reproduced here with the modified bases underlined: SEQ ID No 8:

CUGUUAAUGCUAAUCGUGAUAGGGAUUUUUGCCUCCAACUGAUUCCUACAU
AUUAGCAUUAACAG

Applicant also discovered that changing 5′ “GC” to “AU” (arrow 1 in FIG. 2) in pluripotent cells will favor miR-155-3p strand selection in DA neural cells and will reduce wildtype miR-155-3p strand selection and reduce wildtype miR-155-5p strand selection in DA neural cells. This is shown in SEQ ID No 8 which is reproduced here with the modified bases underlined: SEQ ID No 8:

CUGUUAAUGCUAAUCGUGAUAGGGAUUUUUGCCUCCAACUGAUUCCUACAU
AUUAGCAUUAACAG

Applicant discovered that increasing pyrimidine content in ascending (-5p) in pluripotent cells will favor miR-155-3p strand selection in DA neural cells and will reduce wildtype miR-155-3p strand selection in DA neural cells.

Applicant discovered that increasing pyrimidine content in ascending (-5p) in pluripotent cells will favor miR-155-3p strand selection in DA neural cells and will reduce wildtype miR-155-5p strand selection in DA neural cells.

Applicant discovered that increasing pyrimidine content in ascending (-5p) in pluripotent cells will favor miR-155-3p strand selection in DA neural cells and will reduce wildtype miR-155-3p and reduce wildtype miR-155-5p strand selection in DA neural cells.

Applicant discovered that changing UUAAUG to GGAAUG (5′ nucleotides #1-6) in pluripotent cells will favor miR-155-3p strand selection in DA neural cells and will reduce wildtype miR-155-3p strand selection in DA neural cells.

Applicant discovered that changing UUAAUG to GGAAUG (5′ nucleotides #1-6) in pluripotent cells will favor miR-155-3p strand selection in DA neural cells and will reduce wildtype miR-155-5p strand selection in DA neural cells.

Applicant discovered that changing UUAAUG to GGAAUG (5′ nucleotides #1-6) in pluripotent cells will favor miR-155-3p strand selection in DA neural cells and will reduce wildtype miR-155-3p and reduce wildtype miR-155-5p strand selection in DA neural cells.

The above edits in pluripotent cells may allow for miR-155-3p biased pluripotent cells pluripotent cells. The above edits in pluripotent cells may allow for miR-155-5p un-biased pluripotent cells.

The above edits in pluripotent cells may allow for two classes of pluripotent cells: miR-155-3p biased pluripotent cells and miR-155-5p un-biased pluripotent cells.

Methods of disrupting a nucleotide or nucleotide-editing techniques are known in the art and include, but are not limited to, CRISPR-CAS13 (that recognizes and cuts RNA rather than DNA), TALENS, Zinc-fingers, CRISPR-Cas13 based systems, and CRISPR-Cas7-11. Any method of disrupting a gene sequence known in the art may be used to make the above modifications to miR-155 in pluripotent cells to create miR-155-3p or miR-155-5p biased DA neural cells.

Compositions of Modified iPSCs

Disclosed are compositions comprising modified pluripotent stem cells. In some embodiments, the modified pluripotent stem cells comprise a modification to miR-155-5p. In some embodiments, the modified pluripotent stem cells comprise a modification to miR-155-5p wherein the modification to the miR-155-5p comprises changing 5′ nucleotides 1-2. In some embodiments, the modified pluripotent stem cells comprise a modification to miR-155-5p wherein the modification to the miR-155-5p comprises changing 5′ nucleotides 1-6. In some embodiments, the modified pluripotent stem cells comprise a modification to miR-155-5p wherein the modification to the miR-155-5p comprises changing 5′ nucleotides UUAAUG to GGAAUG.

In some embodiments, the modified pluripotent stem cells comprise a modification to miR-155-3p. In some embodiments, the modified pluripotent stem cells comprise a modification to miR-155-3p wherein the modification to the miR-155-3p comprises changing 5′ nucleotides 1-2. In some embodiments, the modified pluripotent stem cells comprise a modification to miR-155-3p wherein the modification to the miR-155-3p comprises changing 5′ nucleotide 1. In some embodiments, the modified pluripotent stem cells comprise a modification to miR-155-3p wherein the modification to the miR-155-3p comprises changing 5′ nucleotides C to U.

In some embodiments, the modified pluripotent stem cells comprise a modification to miR-155-3p and miR-155-5p. In some embodiments, the modified pluripotent stem cells comprise a modification to miR-155-3p wherein the modification to the miR-155-3p comprises changing 5′ nucleotides 1-2 and changing miR-155-5p 5′ nucleotides 1-2. In some embodiments, the modified pluripotent stem cells comprise a modification to miR-155-3p wherein the modification to the miR-155-3p comprises changing 5′ nucleotide 1 and changing miR-155-5p 5′ nucleotides 1-6. In some embodiments, the modified pluripotent stem cells comprise a modification to miR-155-3p wherein the modification to the miR-155-3p comprises changing 5′ nucleotides C to U and wherein the modification to the miR-155-5p comprises changing 5′ nucleotides UUAAUG to GGAAUG.

In some embodiments, the modified pluripotent stem cells comprise a modification to miR-155 stem loop. In some embodiments, the modified pluripotent stem cells comprise a modification to miR-155 stem loop wherein the modification to the miR-155 stem loop comprises changing nucleotide 25 from a G to an A. In some embodiments, the modified pluripotent stem cells comprise a modification to miR-155 stem loop wherein the modification to the miR-155 stem loop comprises changing nucleotide 43 from a C to a U. In some embodiments, the modified pluripotent stem cells comprise a modification to miR-155 stem loop wherein the modification to the miR-155 stem loop comprises changing nucleotide 25 from a G to an A and changing nucleotide 43 from a C to a U.

In some embodiments, the modified pluripotent stem cells comprise a modification to miR-155-3p, miR-155-5p and miR-155 stem loop. In some embodiments, the modified pluripotent stem cells comprise a modification to miR-155-3p wherein the modification to the miR-155-3p comprises changing 5′ nucleotides 1-2 and changing miR-155-5p 5′ nucleotides 1-2 and changing one nucleotide base pair in the stem loop. In some embodiments, the modified pluripotent stem cells comprise a modification to miR-155-3p wherein the modification to the miR-155-3p comprises changing 5′ nucleotide 1 and changing miR-155-5p 5′ nucleotides 1-6 changing one nucleotide base pair in the stem loop. In some embodiments, the modified pluripotent stem cells comprise a modification to miR-155-3p wherein the modification to the miR-155-3p comprises changing 5′ nucleotides C to U, wherein the modification to the miR-155-5p comprises changing 5′ nucleotides UUAAUG to GGAAUG and wherein the modification to the pre-miR-155 stem loop comprises changing nucleotide 25 from a G to an A and changing nucleotide 43 from a C to a U.

Disclosed is the up-regulation of at least miR-155-3p in pluripotent cells, which induces an anti-inflammatory phenotype in DA neural cells. Disclosed is the up-regulation of at least miR-155-3p in pluripotent cells, and differentiation of the modified pluripotent cells to miR-155-3p biased DA neurons with an anti-inflammatory phenotype, and differentiation of the modified pluripotent cells to miR-155-3p biased DA neurons with an anti-inflammatory phenotype.

Disclosed is the down-regulation of at least miR-155-5p in pluripotent cells, which induces an anti-inflammatory phenotype in DA neural cells. Disclosed is the downregulation of at least miR-155-5p in pluripotent cells, and differentiation of the modified pluripotent cells to miR-155-5p un-biased DA neurons with an anti-inflammatory phenotype.

Disclosed is the up-regulation of at least miR-155-3p in pluripotent cells, which induces an anti-inflammatory phenotype in DA neural cells, whilst down-regulation of at least miR-155-5p in pluripotent cells which also induces an anti-inflammatory phenotype in DA neural cells. Disclosed is the up-regulation of at least miR-155-3p in pluripotent cells, and differentiation of the modified pluripotent cells to miR-155-3p biased DA neurons with an anti-inflammatory phenotype, whilst down-regulation of at least miR-155-5p and differentiation of the modified pluripotent cells to miR-155-3p biased DA neurons with an anti-inflammatory phenotype.

Provided is a method of predisposing pluripotent cells to differentiate into miR-155-3p-biased and/or miR-155-5p-un-biased DA neural cells, the method comprising up-regulating a level of at least one exogenous miRNA selected from the group comprising or consisting of miR-155-3p and miR-155-5p in pluripotent cells, thereby predisposing the pluripotent cells to differentiate into cells with an anti-inflammatory or inflammatory phenotype.

In some embodiments, the pluripotent cells are human or rodent. In some embodiments, the pluripotent cells are human. In some embodiments, the pluripotent cells are isolated from placenta and umbilical cord of newborn humans. In some embodiments, the pluripotent cells are isolated from bone marrow of humans. In some embodiments, the pluripotent cells are at least 50% purified, more preferably at least 75% purified and even more preferably at least 90% purified.

Differentiation of iPSCs to DA Neurons

### Isolation of Pluripotent Cells

The embryonic stem cells of some embodiments can be obtained using well-known cell-culture methods. For example, human embryonic stem cells can be isolated from human blastocysts. Human blastocysts are typically obtained from human in vivo pre-implantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., U.S. Pat. No. 5,843,780; Science 282:1145, 1998; Curr. Top. Dev. Biol. 38:133, 1998; Proc. Natl. Acad. Sci. USA 92:7844, 1995; Bongso et al., Hum Reprod 4:706, 30 1989]; and Gardner et al., Fertil. Steril. 69:84, 1998].

It will be appreciated that commercially available stem cells can also be used. Human ES cells can be purchased from the NIH human embryonic stem cells registry (www.escr.nih.gov). Non-limiting examples of commercially available embryonic stem cell lines are BGO1, BG02, BG03, BG04, CY12, CY30, CY92, CY1O, TE03 and TE32.

In addition, ES cells can be obtained from other species as well, including mouse (Mills and Bradley, 2001), golden hamster Doetschman et al., 1988, Dev Biol. 127:224-7], rat Iannaccone et al., 1994, Dev Biol. 163:288-92] rabbit Giles et al. 1993, Mol Reprod Dev. 36:130-8; Graves & Moreadith, 1993, Mol Reprod Dev. 1993, 36:424-33], several domestic animal species Notarianni et al., 1991, J Reprod Fertil Suppl. 43:255-60; Wheeler 1994, Reprod Fertil Dev. 6:563-8; Mitalipova et al., 2001, Cloning. 3:59-67] and non-human primate species (Rhesus monkey and marmoset) Thomson et al., 1995, Proc Natl Acad Sci USA. 92:7844-8; Thomson et al., 1996, Biol Reprod. 55:254-9].

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 Yamanaka S, Cell Stem Cell. 2007, 1 (1): 39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872. Other embryonic-like stem cells can be generated by nuclear transfer to oocytes, fusion with embryonic stem cells or nuclear transfer into zygotes if the recipient cells are arrested in mitosis.

The pluripotent cells (hES or iPSC) used herein may be of autologous, syngeneic or allogeneic related (matched siblings or haploidentical family members) or unrelated fully mismatched source.

### Culturing Pluripotent Cells

Culturing of pluripotent cells can be performed in any media that supports pluripotent cells which are known in the art. Methods for preparing and culturing pluripotent stem cells such as ES cells can be found in standard textbooks and reviews in cell biology, tissue culture, and embryology, including teratocarcinomas and embryonic stem cells: Guide to Techniques in Mouse Development (1993); Embryonic Stem Cell Differentiation in vitro (1993); Properties and uses of Embryonic Stem Cells: Prospects for Application to Human Biology and Gene Therapy (1998), all incorporated herein by reference. Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995).

As mentioned, the cultured pluripotent cells can be modified. The cultured modified pluripotent cells have at least one modified miRNAs such as pre-mir155-5p or pre-mir155-3p. The cultured modified pluripotent cells have at least one modified miRNAs such as pre-mir155-5p or pre-mir155-3p in order to induce differentiation towards miR-155-3p biased or miR-155-5p biased DA neurons. The cultured modified pluripotent cells have at least one modified miRNAs such as pre-mir155-5p or pre-mir155-3p in order to induce differentiation towards miR-155-3p biased and/or miR-155-5p un-biased DA neurons.

### Differentiation Methods

During or following the differentiation step the pluripotent cells may be monitored for their differentiation state. Cell differentiation can be determined upon examination of cell or tissue-specific markers which are known to be indicative of differentiation.

Below is a list of markers that may be used to confirm differentiation into DA neurons: FOXA2 or LMX1. Additional markers include TH, rthodenticle homeobox 2 (OTX2), nuclear receptor related 1 protein (NURR1), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), G-protein coupled, inwardly rectifying potassium channel (Kir3.2/GIRK2), CD 142, DCSM1, CD63, and CD99.

Tissue/cell specific markers can be detected using immunological techniques well known in the art Thomson J A et al., (1998). Science 282:1145-7. Examples include, but are not limited to, flow cytometry for membrane-bound markers, immunohistochemistry for extracellular and intracellular markers and enzymatic immunoassay, for secreted molecular markers.

It will be appreciated that the cells obtained according to the methods described herein may be enriched for a particular cell type—e.g. progenitor cell type or mature cell type. Thus for example, the time of differentiation may be selected to obtain an earlier progenitor type or a later mature cell type.

In some embodiments, the DA neurons produced from differentiating iPCs comprise three distinct cell populations: A9 dopamine neurons, astrocytes and vascular leptomeningeal cells (VLMC). In some embodiments, the A9 dopamine neurons are miR-155-3p-biased. In some embodiments, the A9 dopamine neurons are miR-155-5p-biased. In some embodiments, the A9 dopamine neurons are miR-155-5p-un-biased. In some embodiments, the astrocytes are miR-155-3p-biased. In some embodiments, the astrocytes are miR-155-5p-biased. In some embodiments, the astrocytes are miR-155-5p-un-biased. In some embodiments, the VLMC are miR-155-3p-biased. In some embodiments, the VLMC are miR-155-5p-biased. In some embodiments, the VLMC are miR-155-5p-un-biased.

In some embodiments, the A9 dopamine neurons are miR-155-3p-biased and are isolated from the astrocytes and vascular leptomeningeal cells. In some embodiments, the A9 dopamine neurons are miR-155-5p-biased and are isolated from the astrocytes and vascular leptomeningeal cells. In some embodiments, the A9 dopamine neurons are miR-155-5p-unbiased and are isolated from the astrocytes and vascular leptomeningeal cells. In some embodiments, the astrocytes are miR-155-3p-biased and are isolated from the A9 dopamine neurons and vascular leptomeningeal cells. In some embodiments, the astrocytes are miR-155-5p-biased and are isolated from the A9 dopamine neurons and vascular leptomeningeal cells. In some embodiments, the astrocytes are miR-155-5p-unbiased and are isolated from the A9 dopamine neurons and vascular leptomeningeal cells. In some embodiments, the VLMC are miR-155-3p-biased and are isolated from the A9 dopamine neurons and astrocytes. In some embodiments, the VLMC are miR-155-5p-biased and are isolated from the A9 dopamine neurons and astrocytes. In some embodiments, the VLMC are miR-155-5p-unbiased and are isolated from the A9 dopamine neurons and astrocytes.

Further enrichment of a particular cell type may be affected using cell sorting techniques such as FACS and magnetic sorting.

In addition, cell differentiation can be also followed by specific reporters that are tagged with GFP or RFP and exhibit increased fluorescence upon differentiation.

### Down-Regulation of the Targets

By determining the targets of the miRNAs that are proposed for up-regulation, it will be appreciated that the scope of the disclosure may be broadened to include down-regulation of the targets by means other than contacting with miRNA. Correspondingly, by determining the targets of the miRNAs that are proposed for down-regulation, it will be appreciated that the scope of the disclosure may be broadened to include up-regulation of the targets.

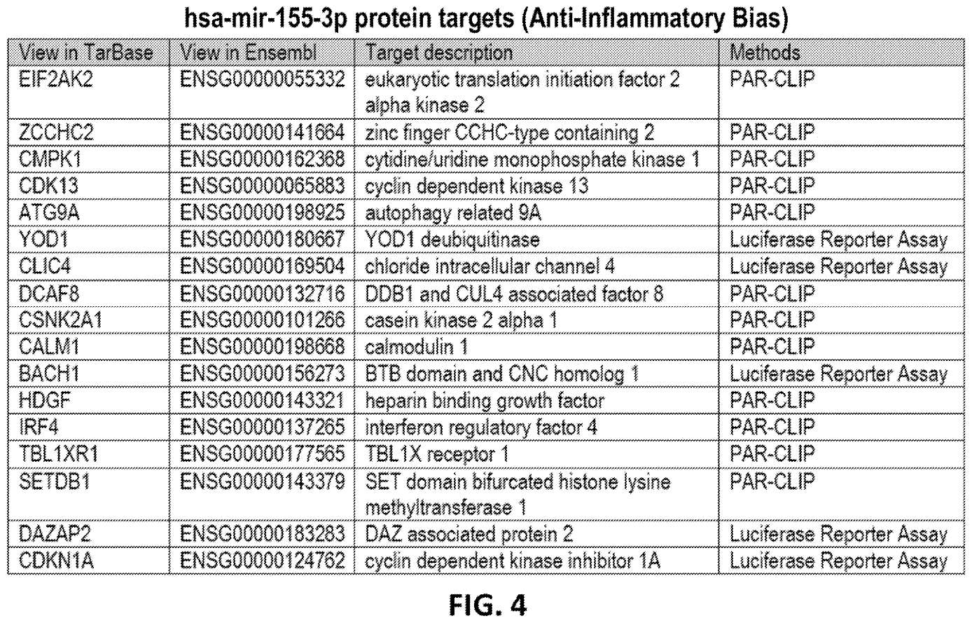

For example, the present inventors contemplate that one of the targets of miR-155-3p is related to the protein targets in FIG. 4. Thus contemplated is that differentiation towards the miR-155-3p biased DA neuron may be affected by down-regulation of these proteins. Contemplates is that differentiation towards the miR-155-3p biased and/or miR-155-5p un-biased DA neuron may be affected by up-regulation of these proteins.

Thus, there is provided a method of generating DA neural cells, the method comprising contacting pluripotent cells with an agent that down-regulates an amount and/or activity of the following protein targets EIF2AK2, ZCCHC2, CMPK1, CDK13, ATG9A, YOD1, CLIC4, DCAF8, CSNK2A1, CALM1, BACH1, HDGF, IRF4, TBL1XR1, SETDB1, DAZAP2, and/or CDKN1A, thereby generating miR-155-3p biased DA neuron cells. Also provided is a method of generating DA neural cells, the method comprising contacting pluripotent cells with an agent that up-regulates an amount and/or activity of the proteins in FIG. 4, thereby generating miR-155-3p biased DA neuron cells.

Thus, there is provided a method of generating DA neural cells, the method comprising contacting pluripotent cells with an agent that down-regulates an amount and/or activity of the following protein targets EIF2AK2, ZCCHC2, CMPK1, CDK13, ATG9A, YOD1, CLIC4, DCAF8, CSNK2A1, CALM1, BACH1, HDGF, IRF4, TBL1XR1, SETDB1, DAZAP2, and/or CDKN1A, thereby generating miR-155-5p unbiased DA neuron cells. Also provided is a method of generating DA neural cells, the method comprising contacting pluripotent cells with an agent that up-regulates an amount and/or activity of the proteins in FIG. 4, thereby generating miR-155-5p unbiased DA neuron cells.

Down-regulation of at least one protein in FIG. 4 (or any of the other miRNA targets) can be obtained at the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide and the like.

One example of an agent capable of down-regulating at least one protein in FIG. 4. is an antibody or antibody fragment capable of specifically binding thereto. Preferably, the antibody is capable of being internalized by the cell and entering the nucleus. The term “antibody” as used herein includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Down-regulation of hsa-miR-155-3p or hsa-miR-155-5p can be also achieved by RNA silencing. As used herein, the phrase “RNA silencing” refers to a group of regulatory mechanisms e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, use of dsRNA to down-regulate protein expression from the mRNA is disclosed.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134.

In particular, disclosed is the introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433, and Diallo et al, Oligonucleotides, Oct. 1, 2003, 13 (5): 381-392. doi: 10.1089/154545703322617069.

Disclosed is the introduction of long dsRNA specifically designed not to induce the interferon and PKR pathways for down-regulating gene expression. For example, Shinagwa and Ishii Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate dsRNA export to the cytoplasm, long ds-RNA from pDECAP does not induce the interferon response.

Introduction of Small Inhibitory RNAs (siRNAs)

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3; (Brummelkamp, T. R. et al. (2002) Science 296:550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

### RNA Silencing Agent

According to another embodiment the RNA silencing agent may be a miRNA, as further described herein above.

Synthesis of RNA silencing agents suitable for use herein can be affected as follows. First, the miRNA target mRNA sequence (e.g. CTGF sequence) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

The RNA silencing agents may comprise nucleic acid analogs that may have at least one different linkage, e.g., phosphoramidate, phosphorothioate, phosphorodithioate, or 0-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-0H-group may be replaced by a group selected from H, OR, R, halo, SH, SR, NH2, NHR, NR2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005), Soutschek et al., Nature 432:173-178 (2004), and U.S. Patent Publication No. 20050107325, which are incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver and kidney. Mixtures of naturally occurring nucleic acids and analogs may be made; alternatively, mixtures of different nucleic acid analogs, and mixtures of naturally occurring nucleic acids and analogs may be made.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides include, but are not limited to, penetratin, transportan, plsl, TAT (48-60), pVEC, MTS, and MAP.

DNAzyme Molecule Capable of Specifically Cleaving an mRNA Transcript

Another agent capable of down-regulating proteins in FIG. 4 is a DNAzyme molecule capable of specifically cleaving an mRNA transcript or DNA sequence of CTGF. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine: pyrimidine junctions (Santoro, S. W. & Joyce, G. F. Proc. Natl, Acad. Sci. USA 20 199; for rev of DNAzymes see Khachigian, L M Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al.

Antisense Polynucleotide Capable of Specifically Hybridizing with an mRNA Transcript Encoding Proteins in FIG. 4

Down-regulation of hsa-miR-155-3p or hsa-miR-155-5p can also be obtained by using an antisense polynucleotide capable of specifically hybridizing with an mRNA transcript encoding hsa-miR-155-3p or hsa-miR-155-5p.

Design of antisense molecules which can be used to efficiently down-regulate hsa-miR-155-3p or hsa-miR-155-5p should take into consideration two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76:75-6 (1998); Kronenwett et al. Blood 91:852-62 (1998); Rajur et al. Bioconjug Chem 8:935-40 (1997); Lavigne et al. Biochem Biophys Res Commun 237:566-71 (1997) and Aoki et al. (1997) Biochem Biophys Res Commun 231:540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65:1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp 130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16:1374-1375 (1998)].

Specifically Cleaving an mRNA Transcript Encoding Hsa-miR-155-3p or Hsa-miR-155-5p

Another agent capable of down-regulating hsa-miR-155-3p or hsa-miR-155-5p is a ribozyme molecule capable of specifically cleaving an mRNA transcript encoding hsa-miR-155-3p or hsa-miR-155-5p. Ribozymes are being increasingly used for the sequence-specific inhibition of gene expression by the cleavage of mRNAs encoding proteins of interest Welch et al., Curr Opin Biotechnol. 9:486-96 (1998)]. The possibility of designing ribozymes to cleave any specific target RNA has rendered them valuable tools in both basic research and therapeutic applications.

### Triplex Forming Oligonuclotides (TFOs).

An additional method of regulating the expression of a hsa-miR-155-3p or hsa-miR-155-5p gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408.

Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

oligo3′--AGGT
duplex5′--AGCT [symbol]
duplex3′--TCGA [symbol]
indicates data missing or illegible when filed

However, it has been shown that the A-AT and G-GC triplets have the greatest triple helical stability (Reither and Jeltsch, BMC Biochem, 2002 Sep. 12, Epub). The same authors have demonstrated that TFOs designed according to the A-AT and G-GC rule do not form non-specific triplexes, indicating that the triplex formation is indeed sequence specific.

Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFG 1 and endogenous HPRT genes in mammalian cells (Vasquez et al., Nucl Acids Res. 1999; 27:1176-81, and Puri, et al, J Biol Chem, 2001; 276:28991-98), and the sequence- and target specific down-regulation of expression of the Ets2 transcription factor, important in prostate cancer etiology (Carbone, et al, Nucl Acid Res. 2003; 31:833-43) and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

### Modification of the Hsa-miR-155-3p or Hsa-miR-155-5p Locus Using CRISPR

In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

Also contemplated is silencing hsa-miR-155-3p or hsa-miR-155-5p by genetic modification of the hsa-miR-155-3p or hsa-miR-155-5p locus. Also contemplated is silencing hsa-miR-155-3p or hsa-miR-155-5p by genetic modification of the hsa-miR-155-BIC locus. This modification can include complete deletion of part or all of the coding region such that no functional protein is produced. Modification can also include mutation or deletion of the part or all of the promotor, such that the coding region is not transcribed. In some embodiments, silencing of hsa-miR-155-3p or hsa-miR-155-5p comprises introduction of CRISPR/Cas13 or CRISPR/Cas7-11 reagents to genetically delete and/or modify the hsa-miR-155-3p or hsa-miR-155-5p genomic locus.

The conditions used for contacting the mesenchymal stem cells are selected for a time period/concentration of cells/concentration of hsa-miR-155-3p or hsa-miR-155-5p down-regulatory agent/ratio between cells and hsa-miR-155-3p or hsa-miR-155-5p down-regulatory agent which enable the hsa-miR-155-3p or hsa-miR-155-5p down-regulatory agent to induce differentiation thereof.

Isolated cell populations obtained according to the methods describe herein are typically non-homogeneous, although homogeneous cell populations are also contemplated.

According to a particular embodiment, the cell populations are genetically modified to express an exogenous miRNA or a polynucleotide agent capable of down-regulating the miRNA.

As mentioned, one of the following miRNAs in pluripotent cells are modified in order to induce differentiation into miR-155-3p biased or miR-155-5p unbiased or miR-155-5p biased DA neurons, miR-155-3p or miR-155-5p. In some embodiments, the modification causes a down-regulation of the particular miRNAs—namely, miR-155-3p or miR-155-5p. In some embodiments, the modification causes an up-regulation of the particular miRNAs—namely, miR-155-3p or miR-155-5p. In some embodiments, the modification causes an up-regulation of the particular miRNAs—namely, miR-155-3p and a down-regulation of the particular miRNAs—namely, miR-155-5p. In some embodiments, the modification causes an up-regulation of the particular miRNAs—namely, miR-155-5p and a down-regulation of the particular miRNAs—namely, miR-155-3p. Down-regulating such miRNAs can be affected using a polynucleotide which is hybridizable in cells under physiological conditions to the miRNA.

In some embodiments, manipulation of particular miRNAs in pluripotent cells, cells expressing miR-155-3p-biased, or miR-155-3p-un-biased or miR-155-5p-biased or miR-155-5p-unbiased DA neural cells may be generated. The miR-155-3p-biased DA neural cells may have an anti-inflammatory effect. The miR-155-5p-biased DA neural cells may have an inflammatory effect. The miR-155-3p-un-biased DA neural cells may have an anti-inflammatory effect. The miR-155-5p-un-biased DA neural cells may have an inflammatory effect.

Provided a method of producing miR-155-3p-biased, or miR-155-3p-unbiased, miR-155-5p-un-biased or miR-155-5p-biased DA neural cells comprising up-regulating a level of at least one exogenous miRNA selected from the group consisting of miR-155-3p or miR-155-5p in pluripotent cells. According to a particular embodiment, the DA neural cells express at least FOXA2 and LMX1.

Provided a method of producing miR-155-3p-biased, miR-155-5p-un-biased DA neural cells comprising up-regulating a level of at least one exogenous miRNA selected from the group consisting of miR-155-3p or miR-155-5p in pluripotent cells. According to a particular embodiment, the DA neural cells express at least FOXA2 and LMX1.

A miRNA array analysis on the differentiated and non-differentiated cells should reveal a number of miRNAs that are overexpressed in a statistically significant manner (more than 3 fold) and a number of miRNAs that were downregulated in a statistically significant manner (more than 3 fold).

Disclosed are methods of ex vivo differentiating pluripotent cells towards neural progenitor cells and DA neurons using microRNAs.

Numerous methods are known in the art for differentiating pluripotent cells to DA neuronal cells including genetic modification and/or culturing in a medium which promotes differentiation towards that fate. The medium typically comprises at least one activator of Sonic hedgehog (SHH) signaling, and at least one activator of wingless (Wnt) signaling. Typically, the differentiation is affected in serum free medium, or serum replacements.

The present disclosure relates to the field of stem cell biology, in particular the lineage specific differentiation of pluripotent or multipotent stem cells, which can include, but is not limited to, human embryonic stem cells (hESC) in addition to nonembryonic induced pluripotent stem cells (iPSC), somatic stem cells, stem cells from patients with a disease, or any other cell capable of lineage specific differentiation. Specifically described are methods to direct the lineage specific differentiation of hESC and/or iPSC into floor plate midbrain progenitor cells and then further into large populations of midbrain fate FOXA2+LMX1A+TH+ dopamine (DA) neurons using novel culture conditions. The midbrain fate FOXA2+LMX1A+TH+ dopamine (DA) neurons made using the methods disclosed herein are further contemplated for various uses including, but not limited to, use in in vitro drug discovery assays, neurology research, and as a therapeutic to reverse disease of, or damage to, a lack of dopamine neurons in a patient. Further, compositions and methods are provided for differentiating midbrain fate FOXA2+LMX1A+TH+ dopamine (DA) neurons from human pluripotent stem cells for use in disease modeling, in particular Parkinson's disease.

As discussed above, differentiation of pluripotent stem cells to DA Neurons is known to those of skill in the art. See U.S. Pat. Nos. 10,280,398 and 10,590,383. Table 1 below is a single representative example of differentiation of pluripotent stem cells to DA Neurons. Table 1 below is a reproduction of Table 6, condition 9 from U.S. Pat. No. 10,590,383. The cell population at day 17 (D17) results in FoxA2+/Lmx1+DA progenitor cells. When the iPSC cells are modified as described herein, the cell population at day 17 (D17) results in FoxA2+/Lmx1+ modified DA progenitor cells.

TABLE 1
1. Condition 9 (200 nM LDN)

| | | | | |
|---|---|---|---|---|
| Component | Vendor | Cat # | Stock | Final Conc. |

E8 Plating Medium (Day −2)

| | | | | | | |
|---|---|---|---|---|---|---|
| Essential 8 Basal Medium | Life Technologies | A14666SA | 1 | x | 98 | % |
| Essential 8 Supplement | Life Technologies | A146665A | 50 | x | 2 | % |
| H1152 | CDI | H024 | 100 | μM | 1 | μM |

E8 Medium (Days −1 and 0)

| | | | | | | |
|---|---|---|---|---|---|---|
| Essential 8 Basal Medium | Life Technologies | A146665A | 1 | x | 98 | % |
| Essential 8 Supplement | Life Technologies | A146665A | 50 | x | 2 | % |

DI DA Induction Medium (Day 1)

| | | | | | | |
|---|---|---|---|---|---|---|
| DMEM/F12 | Life Technologies | 11330-032 | 1 | x | 98 | % |
| B-27 Supplement (+VitA) | Life Technologies | 17504-044 | 2 | x | 2 | % |
| LDN-193189 | Stemgent | 04-0074 | 10 | mM | 200 | nM |
| Purmorphamine | Cayman | 10009634 | 10 | mM | 2 | μM |
| C25II SHH | R&D Systems | 464-SH | 100 | μg/mL | 100 | ng/mL |

D2 DA Induction Medium (Day 2)

| | | | | | | |
|---|---|---|---|---|---|---|
| DMEM/F12 | Life Technologies | 11330-032 | 1 | x | 98 | % |
| B-27 Supplement (+VitA) | Life Technologies | 17504-044 | 2 | x | 2 | % |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |

D3-4 DA Induction Medium (Days 3 and 4)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |
| PD0325901 | Stemgent | 04-0006 | 10 | mM | 1.0 | μM |

DA Quench Medium 1 (Days 5 and 17)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| Blebbistatin | Sigma | B0560 | 10,000 | x | 2.5 | μM |

DS DA Aggregate Formation Medium (Day 5)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |
| PD0325901 | Stemgent | 04-0006 | 10 | mM | 1.0 | μM |
| Blebbistatin | Sigma | B0560 | 2,500 | x | 10 | μM |

D6-7 DA Induction Medium (Day 6-7)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/m | 100 | ng/mL |
| CHIR99021 | Stemgent | 04-0004 | 20 | mM | 1.75 | μM |

D8-10 DA Aggregate Maintenance Medium (Days 8-10)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| CHIR99021 | Stemgent | 04-0004 | 20 | mM | 1.75 | μM |

D11-16 DA Aggregate Maintenance Medium (Days 11-16)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| CHIR99021 | Stemgent | 04-0004 | 20 | mM | 3.0 | μM |
| FGF8 | R&D Systems | 423-F8 | 50 | μg/mL | 100 | ng/mL |

D17 DA Aggregate Plating Medium (Days 17)

| | | | | | | |
|---|---|---|---|---|---|---|
| DMEM/F12 | Life Technologies | 11330-032 | 1 | x | 98 | % |
| B-27 Supplement (−VitA) | Life Technologies | 12587-010 | 2 | x | 2 | % |
| LDN-193189 | Stemgent | 04-0074 | 10 | mM | 200 | nM |
| CHIR99021 | Stemgent | 04-0004 | 20 | mM | 3.0 | μM |
| FGF8 | R&D Systems | 423-F8 | 50 | μg/mL | 100 | ng/ml |

Condition 9 (200 nM LDN)

| | | | | | | |
|---|---|---|---|---|---|---|
| Blebbistatin | Sigma | B0560 | 10,000 | x | 2.5 | μM |
| Neurobasal | Life Technologies | 21103-049 | 1 | x | 98 | % |
| Glutamax | Life Technologies | 35050-061 | 200 | mM | 2 | mM |
| B-27 Supplement (−VitA) | Life Technologies | 12587-010 | 2 | x | 2 | % |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |
| Neurobasal | Life Technologies | 21103-049 | 1 | x | 98 | % |
| Glutamax | Life Technologies | 35050-061 | 200 | mM | 2 | mM |
| B-27 Supplement (−VitA) | Life Technologies | 12587-010 | 2 | x | 2 | % |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |
| Mitomycin C | Sigma | M4287 | 1 | mg/mL | 100 | ng/mL |

DA Quench Medium 2 (Days 18+)

| | | | | | | |
|---|---|---|---|---|---|---|
| Neurobasal | Life Technologies | 21103-049 | 1 | x | 98 | % |
| Glutamax | Life Technologies | 35050-061 | 200 | mM | 2 | mM |
| B-27 Supplement (−VitA) | Life Technologies | 12587-010 | 2 | x | 2 | % |
| Blebbistatin | Sigma | B0560 | 10,000 | x | 2.5 | μM |

DA Maturation Plating Medium (Days 18+)

| | | | | | | |
|---|---|---|---|---|---|---|
| Neurobasal | Life Technologies | 21103-049 | 1 | x | 98 | % |
| Glutamax | Life Technologies | 35050-061 | 200 | mM | 2 | mM |
| B-27 Supplement (−VitA) | Life Technologies | 12587-010 | 2 | x | 2 | % |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/ml |
| dbcAMP | Sigma | D0627 | 100 | mM | 500 | μM |
| DAPT | Sigma | D5942 | 20 | mM | 5 | μM |
| Blebbistatin | Sigma | B0560 | 10,000 | x | 2.5 | μM |

The D17 cells consist of three distinct cell populations: A9 DN (majority), astrocytes and VLMC.

When miR-155 is modified in the pluripotent cells, it produces a D17 cell population with miRNA having an anti-inflammatory effect. When miR-155 is modified in the pluripotent cells, it produces a D17 cell population with miRNA having an pro-inflammatory effect. When miR-155 is modified in the pluripotent cells, it produces a D17 cell population with miRNA having an miR-155-3p bias. When miR-155 is modified in the pluripotent cells, it produces a D17 cell population with miRNA having an miR-155-5p bias.

The disclosure can be further understood by the following numbered paragraphs

Paragraph 1. A method of promoting pluripotent cell differentiation toward a miR-155-3p-biased or miR-155-5p-biased DA neural cell, the method comprising:
(i) introducing into said pluripotent cells the following exogenous microRNAs (miR): miR-155-3p or miR-155-5p; and
(ii) confirming increased expression of at least a miR-155-3p or a miR-155-5p, thereby promoting differentiation of the pluripotent cell differentiation toward a miR-155-3p-biased or miR-155-5p-biased DA neural cell.

Paragraph 2. The method of Paragraph 1, wherein said introducing comprises any one of:
(iii) transfecting said pluripotent cell with an expression vector which comprises a polynucleotide sequence which encodes a pre-miRNA of said miR; or
(iv) transfecting said pluripotent cell with an expression vector which comprises a polynucleotide sequence which encodes said miR.

Paragraph 3. The method of Paragraph 1, further comprising introducing into said pluripotent cell a miR-155-3p antagomir or a miR-15505p antagomir, before said confirming.

Paragraph 4. A method of promoting pluripotent cell differentiation toward a miR-155-3p-biased or miR-155-5p-biased DA neuron cell, the method comprising:
(v) introducing into said pluripotent cell the following exogenous microRNAs (miR): miR-155-3p, or miR-155-5p; and
(vi) confirming expression of at least one DA neuron marker selected from the group comprising FOXA2 or LMX1, wherein said expressing results in at least 50% of the DA neuron cells having increased expression of miR-155-3p, or miR-155-5p compared to wildtype.

Paragraph 5. The method of Paragraph 4, wherein said introducing comprises any one of:
(vii) transfecting said pluripotent cell with an expression vector which comprises a polynucleotide sequence which encodes a pre-miRNA of said miR; or
(viii) transfecting said pluripotent cell with an expression vector which comprises a polynucleotide sequence which encodes said miR.

Paragraph 6. The method of Paragraph 4, further comprising introducing into said pluripotent cell a miR-155-3p antagomir or a miR-155-5p antagomir, before said confirming.

### DA Neural Cell Populations

Provided herein is a purified or isolated population of cells differentiated from pluripotent cells.

Disclosed is a composition comprising DA neurons, the DA neurons having modified pre-miR-155. Disclosed is a composition comprising DA neurons, the DA neurons having complete complementarity with the wildtype miRNA sequence except for 1, 2, or 3 nucleotide substitutions, terminal additions, and/or truncations.

Disclosed is a composition comprising DA neurons, the DA neurons having a modified pre-miR-155 having SEQ ID No: 6. Disclosed is a composition comprising DA neurons, the DA neurons having a modified pre-miR-155 having SEQ ID No: 7. Disclosed is a composition comprising DA neurons, the DA neurons having a modified pre-miR-155 stem loop having SEQ ID No: 8. Disclosed is a composition comprising DA neurons, the DA neurons having a modified pre-miR-155 having SEQ ID No: 6 and SEQ ID No: 7. Disclosed is a composition comprising DA neurons, the DA neurons having a modified pre-miR-155 having SEQ ID No: 6, and SEQ ID No: 7, and a pre-miR-155 stem loop having SEQ ID No: 8. Disclosed is a composition comprising DA neurons, the DA neurons having a modified pre-miR-155 having SEQ ID No: 6 and a pre-miR-155 stem loop having SEQ ID No: 8. Disclosed is a composition comprising DA neurons, the DA neurons having a modified pre-miR-155 having SEQ ID No: 7 and a pre-miR-155 stem loop having SEQ ID No: 8. SEQ ID Nos: 6-8 can be used to generate miR-155-3p biased or miR-155-5p biased DA neurons. Disclosed is a composition comprising DA neurons, the DA neurons having a modified pre-miR-155 having SEQ ID No: 7 and a pre-miR-155 stem loop having SEQ ID No: 8. SEQ ID Nos: 6-8 can be used to generate miR-155-3p biased and miR-155-5p un-biased DA neurons.

Disclosed is a composition comprising DA neurons, the DA neurons having a modified pre-miR-155, which favors miR-155-3p strand selection. Disclosed is a composition comprising DA neurons, the DA neurons having a modified pre-miR-155, which favors miR-155-5p strand selection. Disclosed is a composition comprising DA neurons, the DA neurons having a modified pre-miR-155, which decreases miR-155-3p strand selection. Disclosed is a composition comprising DA neurons, the DA neurons having a modified pre-miR-155, which decreases miR-155-5p strand selection. Disclosed is a composition comprising DA neurons, the DA neurons having a modified pre-miR-155, which favors miR-155-3p strand selection and decreasesmiR-155-5p strand selection.

In certain aspects, disclosed is a pre-miRNA-155 sequence, wherein the pre-miRNA-155 incorporated into a DA neuronal cell has an anti-inflammatory effect on human subjects. In certain aspects, disclosed is a pre-miRNA-155 sequence, wherein the pre-miRNA-155 incorporated into a DA neuronal cell has an anti-inflammatory effect on human subjects. In certain aspects, disclosed is a pre-miRNA-155 sequence, wherein the pre-miRNA-155 incorporated into a DA neuronal cell has a pro-inflammatory effect on human subjects.

These compositions are useful for the treatment of disease, such as neurologic disease, PD and associated disorders.

This disclosure also provides pharmaceutical compositions comprising, or consisting essentially of, or yet further consisting of, purified or isolated miR-155-3p biased DA cell populations or miR-155-5p biased DA cell populations. In one aspect, the pharmaceutical composition comprises, or alternatively consists essentially of, or yet further consists of, a pharmaceutically acceptable carrier and an effective amount of these miR-155-3p biased DA cell populations or miR-155-5p biased DA cell populations.

This disclosure also provides pharmaceutical compositions comprising, or consisting essentially of, or yet further consisting of, purified or isolated miR-155-3p un-biased DA cell populations or miR-155-5p un-biased DA cell populations. In one aspect, the pharmaceutical composition comprises, or alternatively consists essentially of, or yet further consists of, a pharmaceutically acceptable carrier and an effective amount of these miR-155-3p biased DA cell populations or miR-155-5p biased DA cell populations.

Non-limiting examples of carriers include phosphate buffered saline (PBS), saline or a biocompatible matrix material such as a collagen matrix. The compositions can optionally contain a protease inhibitor, glycerol and/or dimethyl sulfoxide (DMSO). The pharmaceutically acceptable carrier comprises one or more of a biocompatible matrix or a liquid carrier. The pharmaceutical compositions of this disclosure can be formulated for freeze-drying or lyophilization using methods known in the art.

The pharmaceutical composition are intended for in vitro and in vivo use. The compositions can comprise a concentration of miR-155-3p biased DA cell populations or miR-155-5p biased DA cell populations from about 1 mg/ml to about 10 mg/ml, or alternatively from about 1 to about 8 mg/ml, or alternatively from 2 to about 8 mg/ml, or alternatively from 2 to about 5 mg/ml, or about 2 to 4 mg/ml, or alternatively from 3 mg/ml to 20 mg/ml. When administered to the subject, an effective amount of the miR-155-3p biased DA cell populations or miR-155-5p biased DA cell populations are administered to the subject, to cause at least about 5%, or alternatively at least about 10%, or alternatively at least about 20%, or alternatively at least about 30%, or alternatively at least about 40%, or alternatively at least about 50%, or alternatively at least about 60%, or alternatively at least about 70%, or alternatively at least about 80%, or alternatively at least about 85%, or alternatively at least about 90%, or alternatively at least about 95%, or alternatively at least about 99% effectiveness in the methods provided herein as compared to a control that does not receive the composition. Comparative effectiveness can be determined by suitable in vitro or in vivo methods as known in the art and briefly exemplified herein.

In one aspect, the compositions are pharmaceutical formulations for use in the therapeutic methods of this disclosure and for the treatment of the appropriate or relevant disease. While the examples are noted for the treatment of PD, the principles can be applied to other disease conditions, including neurologic diseases.

In a further aspect, the disclosure provides a pharmaceutical composition comprising, or alternatively consisting essentially of, or yet further consisting of, the isolated or purified miR-155-3p biased DA cell populations or miR-155-5p biased DA cell populations in a concentration such that composition comprises at least 75%, or alternatively at least 80%, or alternatively at least 85%, or alternatively at least 90%, or alternatively at least 95% or alternatively at least 97%, or alternatively at least 98%, or alternatively, at least 99% of cells in the total composition.

In some embodiments, the pluripotent cells are human.

According to another embodiment, the pluripotent cells are isolated from placenta and umbilical cord of newborn humans.

Cell populations may be selected such that more than about 50% (alternatively more than about 60%, more than about 70%, more than about 80%, more than about 90% or even more than about 95%) of the cells express at least one, at least two, at least three, at least four, at least five of the markers for DA neurons or at least one, at least two, at least three, at least four, at least five of the markers for DA neural cells. DA neural cell markers include but are not limited to: FOXA2, LMX1A, NURR1, TH, OTX2, Tujl, TTF3, PITX3, ASCL, EBF-1, EBF-3, TTR, DAT, Kir3.2/GIRK2, CD 142, DCSM1, CD63, and CD99

The pluripotent cells of used herein may be of autologous, syngeneic or allogeneic related (matched siblings or haploidentical family members) or unrelated fully mismatched source.

Isolated cell populations obtained according to the methods describe herein are typically non-homogeneous, although homogeneous cell populations are also contemplated.

According to a particular embodiment, the cell populations are genetically modified to express an exogenous miRNA or a polynucleotide agent capable of down-regulating the miRNA. In some embodiments, the modified miRNA comprises SEQ ID Nos. 6, 7 or 8. In some embodiments, the modified miRNA comprises a modification of miR-155-3p or miR-155-5p in a pluripotent cell population.

Cell populations may be selected such that more than about 50% (alternatively more than about 60%, more than about 70%, more than about 80%, more than about 90% or even more than about 95%) of the cells have a modified miRNA such as miR-155-3p or miR-155-5p.

Isolation of particular subpopulations of cells may be affected using techniques known in the art including fluorescent activated cell sorting and/or magnetic separation of cells.

The cell populations may comprise DA neurons or DA neuronal cell phenotypes including a cell size, a cell shape, an organelle size and an organelle number. These structural phenotypes may be analyzed using microscopic techniques (e.g. scanning electron microscopy). Antibodies or dyes may be used to highlight distinguishing features in order to aid in the analysis.

The cell populations may be useful for a variety of therapeutic purposes. Representative examples of CNS diseases or disorders that can be beneficially treated with the cells described herein include, but are not limited to, a pain disorder, a motion disorder, a dissociative disorder, a mood disorder, an affective disorder, a neurodegenerative disease or disorder, psychiatric disorders and a convulsive disorder.

More specific examples of such conditions include, but are not limited to, Parkinson's disease, ALS, Multiple Sclerosis, Huntingdon's disease, autoimmune encephalomyelitis, spinal cord injury, cerebral palsy, diabetic neuropathy, glaucatomus neuropathy, macular degeneration, action tremors and tardive dyskinesia, panic, anxiety, depression, alcoholism, insomnia, manic behavior, schizophrenia, autism-spectrum disorder, manic-depressive disorders, Alzheimer's and epilepsy.

### Isolation of Particular Subpopulations of Cells

Isolation of particular subpopulations of cells may be affected using techniques known in the art including fluorescent activated cell sorting and/or magnetic separation of cells.

Differentiation to miR-155-3p or miR-155-3p enhanced DA Neurons can be further understood by the following numbered paragraphs

Paragraph 1: An in vitro method for preparing a cell composition comprising human cells that express both forkhead box protein A2 (FOXA2) and LIM homeobox transcription factor 1 (LMX1) (FOXA2⁺/LMX1⁺ cells) comprising:
Modifying miR-155 in pluripotent cells;
culturing the modified pluripotent cells in the presence of the following signaling modulators:
   (a) a single inhibitor of Small Mothers Against Decapentaplegic (SMAD) signaling,
   (b) at least one activator of Sonic hedgehog (SHH) signaling, and
   (c) at least one activator of wingless (Wnt) signaling;
and culturing said cells in the presence of said modulators for a period of time sufficient to provide a cell composition comprising said FOXA2⁺/LMX1⁺ cells;
wherein the culturing does not comprise culturing the human pluripotent cells in the presence of a second inhibitor of Small Mothers Against Decapentaplegic (SMAD) signaling.

Paragraph 2: An in vitro method for differentiating pluripotent cells into midbrain floor plate precursors, the method comprising:
Modifying miR-155 in pluripotent cells;
exposing a plurality of pluripotent cells to: (a) at least one inhibitor of Small Mothers Against Decapentaplegic (SMAD) signaling, (b) at least one activator of Sonic hedgehog (SHH) signaling and (c) at least one activator of wingless (Wnt) signaling, wherein:
exposure to the at least one inhibitor of SMAD signaling begins on day 0;
said pluripotent cells are exposed to the at least one activator of Wnt signaling 3 days after initiation of exposure to the at least one inhibitor of SMAD signaling;
said pluripotent cells are exposed to the at least one inhibitor of SMAD signaling, the at least one activator of SHH signaling, and the at least one activator of Wnt signaling in amounts effective to produce a plurality of midbrain floor plate precursor cells, at least 10% of which express both forkhead box protein A2 (FOXA2) and LIM homeobox transcription factor 1 alpha (LMX1A); and
optionally further subjecting said plurality of differentiated midbrain floor plate precursor cells to conditions favoring maturation of the midbrain floor plate precursor cells into dopamine neurons.

Paragraph 3: An in vitro method for differentiating pluripotent stem cells, comprising Modifying miR-155 in pluripotent cells; exposing a plurality of modified pluripotent stem cells to at least one inhibitor of Small Mothers Against Decapentaplegic (SMAD) signaling, and exposing the cells to at least one activator of Sonic hedgehog (SHH) signaling and at least one activator of wingless (Wnt) signaling, wherein the cells are exposed to the at least one activator of Wnt signaling three (3) days from the initial exposure of the cells to the at least one inhibitor of SMAD signaling to obtain a cell population comprising at least about 10% differentiated cells expressing both forkhead box protein A2 (FOXA2) and LIM homeobox transcription factor 1 alpha (LMX1A).

Paragraph 4: An in vitro method for differentiating pluripotent stem cells, comprising Modifying miR-155 in pluripotent cells; exposing a plurality of modified pluripotent stem cells to at least one inhibitor of Small Mothers Against Decapentaplegic (SMAD) signaling to obtain a cell population comprising differentiated cells expressing both forkhead box protein A2 (FOXA2) and LIM homeobox transcription factor 1 alpha (LMX1A).

Paragraph 5: The method of any one of paragraphs 1-4, wherein modifying miR-155 in pluripotent cells comprises modifying the miR-155-5p or miR-155-3p.

Paragraph 6: The method of any one of paragraphs 1-4, wherein modifying miR-155 in pluripotent cells comprises modifying the miR-155-5p.

Paragraph 6.1: The method of any one of paragraphs 1-4, wherein modifying miR-155 in pluripotent cells comprises modifying the miR-155-3p.

Paragraph 7: The method of any one of paragraphs 1-4, wherein the FOXA2⁺/LMX1⁺ cells are 155-5p-biased or miR-155-3p-biased.

Paragraph 8: The method of any one of paragraphs 1-4, wherein the FOXA2⁺/LMX1⁺ cells are 155-5p-biased.

Paragraph 9: The method of any one of paragraphs 1-4, wherein the FOXA2⁺/LMX1⁺ cells are miR-155-3p-biased.

Paragraph 10: The method of any one of paragraphs 1-4, wherein the FOXA2⁺/LMX1⁺ cells are miR-155-5p-biased and comprise dopamine neurons, Astrocytes, and VLMC.

Paragraph 11: The method of any one of paragraphs 1-4, wherein the FOXA2⁺/LMX1⁺ cells are miR-155-3p-biased and comprise dopamine neurons, Astrocytes, and VLMC.

Paragraph 12: The method of any one of paragraphs 1-4, wherein the FOXA2⁺/LMX1⁺ cells are miR-155-5p-biased and comprise dopamine neurons, Astrocytes, or VLMC.

Paragraph 13: The method of any one of paragraphs 1-4, wherein the FOXA2⁺/LMX1⁺ cells are miR-155-3p-biased and comprise dopamine neurons, Astrocytes, or VLMC.

Paragraph 14: The method of any one of paragraphs 1-4, wherein the FOXA2⁺/LMX1⁺ cells are miR-155-5p-biased and expression of miR-155-5p is greater in the dopamine neurons compared to the Astrocytes, or VLMC.

Paragraph 15: The method of any one of paragraphs 1-4, wherein the FOXA2⁺/LMX1⁺ cells are miR-155-3p-biased and expression of miR-155-3p is greater in the dopamine neurons compared to the Astrocytes, or VLMC.

Paragraph 16: The method of any one of paragraphs 1-4, wherein the FOXA2⁺/LMX1⁺ cells are miR-155-5p-biased and expression of miR-155-5p is greater in the Astrocytes compared to the dopamine neurons, or VLMC.

Paragraph 17: The method of any one of paragraphs 1-4, wherein the FOXA2⁺/LMX1⁺ cells are miR-155-3p-biased and expression of miR-155-3p is greater in the Astrocytes compared to the dopamine neurons, or VLMC.

Paragraph 18: The method of any one of paragraphs 1-4, wherein the FOXA2⁺/LMX1⁺ cells are miR-155-5p-biased and expression of miR-155-5p is greater in the VLMC compared to the dopamine neurons, or Astrocytes cells.

Paragraph 19: The method of any one of paragraphs 1-4, wherein the FOXA2⁺/LMX1⁺ cells are miR-155-3p-biased and expression of miR-155-3p is greater in the VLMC compared to the dopamine neurons, or Astrocytes cells.

Paragraph 20: The method of any one of paragraphs 1-4, wherein modifying miR-155 in pluripotent cells comprises modifying miR-155-3p to have Seq ID no 7.

Paragraph 21: The method of any one of paragraphs 1-4, wherein modifying miR-155 in pluripotent cells comprises modifying miR-155-5p to have Seq ID no 6.

Paragraph 22: The method of any one of paragraphs 1-4, wherein modifying miR-155 in pluripotent cells comprises modifying miR-155-5p to have Seq ID no 8.

Paragraph 23: The method of any one of paragraphs 1-4, wherein modifying miR-155 in pluripotent cells comprises modifying miR-155-3p to have Seq ID no 8.

### Diagnostic Methods

Disclosed are compositions and methods for the use of miRNAs as a diagnostic tool.

In some embodiments, disclosed is a method of screening a test compound comprising: (a) contacting a miR-155-3p biased DA cell population or miR-155-5p biased DA cell population with the test compound, and (b) measuring the function, physiology, or viability of the cells. In some embodiments, said measuring comprises testing for a toxicological response or an altered electrophysiological response of the cells. In some embodiments, said measuring comprises testing for the differentiation potential of the miR-155-3p biased or miR-155-5p biased DA neural cells.

In some embodiments, a subject's health can be monitored by determining the expression level of at least one anti-inflammatory marker.

The measurement of the above-noted miRNA markers can be combined with clinical parameters. Without being limited, exemplary clinical tests for detecting and diagnosing Parkinson's disease include without limitation: PGA index, FIB-4 index, Fibrometer, FibroSure, Act-test, SAFE, Heapscore, FibroQ, AAR, APRI, CDS, API, Pohls score, Loks model, liver biopsy, ultrasonography, computed tomography, ultrasound elastography, and magnetic resonance elastography.

In some embodiments, when a subject has high levels of miR-155-3p biased DA neural cells or miR-155-5p biased DA neural cells the subject is likely to have a neurologic disease or an associated disorder, and if the exosome expression is normal or reversed from the above, the subject is not likely to have a neurologic disease, or an associated disorder, and therefore therapy is not needed. If the subject is in need of therapy, the miR-155-3p biased DA cell compositions, alone or in combination with other known therapies, can then be administered to the subject in need of treatment. The diagnostic methods can be repeated throughout and after therapy to monitor the subject's health status and the efficacy of the therapy.

The therapy and patient's health can be monitored by determining the level of one or more, two or more, three or more, or all of pro-inflammatory or anti-inflammatory markers in a sample isolated from the patient prior to, during and after the therapy.

Measurement of expression level or activity level can be accomplished by methods known in the art and briefly described herein, e.g., by PCR, qPCR, miRNA arrays, RNA-seq, multiplex miRNA profiling. The tools and methodologies are known in the art and commercially available from various suppliers. The measurement can be compared to suitable controls, e.g., a prior measurement for that subject or a suitable internal control.

Collection of samples of cell populations from body fluid, e.g., urine, blood, saliva, breast milk, lymphatic fluid, serum or plasma can be done with methods known in the art and described briefly herein. The cell populations can be purified from the fluid using the methods disclosed herein in art-recognized methods, such as by ultracentrifugation as described by Thery et al. (2006) “Isolation and characterization of cell populations from cell culture supernatants and biological fluids” Curr. Protoc. Cell Biol., Chapter 3, or as disclosed in Hong et al. (2014) PLOS One 9 (8): e103310, doe: 10,1371 and Jayachandran et al. (2012) J. Immun. Methods, 375:207-214. Commercial kits also are available, e.g., PureExo (101BIO, Palo Alto Calif., for serum and plasma), Exo MIRMIR Plus (Bioo Scientific, Austin Tex., USA), ExoQuick (SBI, Mountain View, Calif., USA, for tissue culture) and Exo-Spin Kit (Cell Guidance Systems, Carlsbad Calif., USA). As apparent to the skilled artisan, the isolation method will depend on the size and composition of the cell to be isolated. As an example, ultracentrifugation can be used for larger cells, and the speed shall not exceed about 70,000 g or alternatively about 60,000 g. Alternatively, ultracentrifugation is used for smaller cell populations, but being much smaller, speeds of 90,000 or alternatively of 100,000 g or more are needed.

The methods are useful in the diagnosis of a subject, e.g., a mammal, an animal, or yet further a human patient. For the purpose of illustration only, a mammal includes but is not limited to a human, a simian, a murine, a rat, a bovine, a canine, a feline, an equine, a porcine or an ovine.

In one aspect, the non-diseased subject is one that is not suffering from a neurologic disease or an associated disorder. In one aspect, the non-diseased subject is one that has an up-regulation of miR-155-3p, as compared to the miR-155-3p profile of a subject that is suffering from a neurologic disease or an associated disorder.

Also provided is a purified or isolated population of cells isolated from a body fluid of a non-diseased subject, wherein the microRNA (miR) profile of the cell populations comprises, or alternatively consist essentially of, or yet further consist of, up-regulation of miR-155-3p, as compared to the miR profile of a subject that is suffering a neurologic disease or an associated disorder.

### Therapeutic Methods

When a marker is used as a basis for selecting a patient for a treatment described herein, the marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

In therapeutic terms, miRNA expression modulation could also enable the development of new treatments.

In some embodiments, a method of inhibiting an inflammatory response in a human subject is disclosed.

Disclosed is a method to treat Parkinson's Disease in a subject in need thereof comprising administering to a subject in need thereof a therapeutic amount of DA neurons with a modified MIR-155. A method to treat Parkinson's Disease in a subject in need thereof comprising administering to a subject in need thereof a therapeutic amount of miR-155-3p biased DA neurons. A method to treat Parkinson's Disease in a subject in need thereof comprising administering to a subject in need thereof a therapeutic amount of miR-155-5p biased DA neurons.

Disclosed is a method to rescue or increase survival of dopamine neurons in a subject in need thereof comprising administering to a subject in need thereof a therapeutic amount of DA neurons with a modified MIR-155. Disclosed is a method to rescue or increase survival of dopamine neurons in a subject in need thereof comprising administering to a subject in need thereof a therapeutic amount of miR-155-3p biased DA neurons. Disclosed is a method to rescue or increase survival of dopamine neurons in a subject in need thereof comprising administering to a subject in need thereof a therapeutic amount of miR-155-5p biased DA neurons.

The compositions are useful for the preparation of a medicament and/or to perform methods for one or more of: a) inhibiting the progression of, b) preventing or c) treating, a disease, e.g., a neurologic disease or an associated disorder.

In one aspect, the compositions are useful for the preparation of a medicament and/or to perform methods for one or more of: a) inhibiting the progression of, b) preventing or c) treating, Parkinson's disease or an associated disorder in a subject in need thereof. The methods comprise, or alternatively consist essentially of, or yet further consist of, administering to the subject an effective amount of the pharmaceutical composition described above including miR-155-3p biased DA neural cells or miR-155-5p biased DA neural cells.

The therapy and patient's health can be monitored using the diagnostic methods disclosed herein. A non-limiting example of such is by determining the level of one, or two or more, three or more, or all of miR-155-3p biased cells and/or miR-155-5p biased cells in a sample isolated from the patient prior to, during and after the therapy.

The therapy and patient's health can be monitored by determining the level of miR-155-3p biased cells and/or miR-155-5p. The therapy and patient's health can be monitored by determining the level of miR-155-3p biased cells and/or miR-155-5p compared to wildtype.

The compositions can then be administered to subjects identified as likely to have Parkinson's disease or an associated disorder. In these methods, the cell populations are allogeneic or autologous to the subject receiving the cell populations.

As noted, the composition is administered in an effective amount. For example, an effective amount comprises from about 1 to about 1,000 mg/kg, or alternatively from about 1 to about 500 mg/kg, or alternatively from about 5 to about 500 mg/kg, or alternatively from about 10 to about 100 mg/kg, or alternatively from about 5 mg/kg to about 100 mg/kg, or alternatively from about 10 mg/kg to about 80 mg/kg, or alternatively from about 10 mg/kg to about 50 mg/kg, or alternatively from about 15 mg/kg to about 50 mg/kg, or alternatively more than 5 mg/kg, or alternatively more than about 10 mg/kg, or alternatively more than about 15 mg/kg, or alternatively more than about 20 mg/kg, or alternatively more than 25 mg/kg, or alternatively more than 30 mg/kg, each as measured per kg of body weight of the subject. The effective amount is in one aspect, per dose, or as a daily dose, or alternatively the total over the course of treatment.

The compositions can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material. The compositions can optionally contain a protease inhibitor, glycerol and/or dimethyl sulfoxide (DMSO).

The pharmaceutical compositions can be conveniently presented in dosage unit form and can be prepared by any of the methods well known in the art of pharmacy. In the pharmaceutical composition the active object compound is included in an amount sufficient to produce the desired therapeutic effect. For example, pharmaceutical compositions of the disclosure may take a form suitable for virtually any mode of administration, including, for example, topical, ocular, oral, buccal, systemic, nasal, injection, transdermal, rectal, and vaginal, or a form suitable for administration by inhalation or insufflation.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

The compositions will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

The amount of compound administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the disclosure will also depend on the age, weight, general health, and severity of the condition of the individual being treated. Dosage may also need to be tailored to the sex of the individual and/or the lung capacity of the individual, where administered by inhalation. Dosage, and frequency of administration of the compositions will also depend on whether the compositions are formulated for treatment of acute episodes of a condition or for the prophylactic treatment of a disorder. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a therapeutic concentration and/or dosage of the miR-155-3p biased cell composition or miR-155-5p biased cell composition, as measured in an in vitro assay. Calculating dosages to achieve such effective dosages for other animal models or human patients is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 1000 mg/kg/day, but can be higher or lower, depending upon, among other factors, the activity of the composition, its bioavailability, the mode of administration, and various factors discussed above. Dosage amount and interval can be adjusted individually to provide local and/or systemic concentration of the cell populations that are sufficient to maintain therapeutic or prophylactic effect. For example, the compositions can be administered once per week, several times per week (e.g., every other day), once per day, or multiple times per day, depending upon, among other things, the mode of administration, the specific indication being treated, and the judgment of the prescribing physician. Skilled artisans will be able to optimize effective local dosages without undue experimentation.

Preferably, the compound(s) will provide therapeutic or prophylactic benefit without causing substantial toxicity. Toxicity of the miR-155-3p biased cells composition or miR-155-5p biased cells composition can be determined using standard pharmaceutical procedures. The dose ratio between toxic and therapeutic (or prophylactic) effect is the therapeutic index. Compositions that exhibit high therapeutic indices are preferred.

The cell populations may be useful for a variety of therapeutic purposes. Representative examples of CNS diseases or disorders that can be beneficially treated with the cells described herein include, but are not limited to, a pain disorder, a motion disorder, a dissociative disorder, a mood disorder, an affective disorder, a neurodegenerative disease or disorder, psychiatric disorders and a convulsive disorder.

More specific examples of such conditions include, but are not limited to, Parkinson's disease, ALS, Multiple Sclerosis, Huntingdon's disease, autoimmune encephalomyelitis, spinal cord injury, cerebral palsy, diabetic neuropathy, glaucatomus neuropathy, macular degeneration, action tremors and tardive dyskinesia, panic, anxiety, depression, alcoholism, insomnia, manic behavior, schizophrenia, autism-spectrum disorder, manic-depressive disorders, Alzheimer's and epilepsy.

The use of differentiated miR-155 modified pluripotent cells may be also indicated for treatment of traumatic lesions of the nervous system including spinal cord injury and also for treatment of stroke caused by bleeding or thrombosis or embolism because of the need to induce neurogenesis and provide survival factors to minimize insult to damaged neurons.

In any of the methods described herein the cells may be obtained from an autologous, semi-allogeneic or non-autologous (i.e., allogeneic or xenogeneic) human donor or embryo or cord/placenta. For example, cells may be isolated from a human cadaver or a donor subject.

The term semi-allogeneic refers to donor cells which are partially-mismatched to recipient cells at a major histocompatibility complex (MHC) class I or class II locus.

The disclosed miR-155-3p or miR-155-5p biased DA neural cells can be administered to the treated individual using a variety of transplantation approaches, the nature of which depends on the site of implantation.

The term or phrase “transplantation”, “cell replacement” or “grafting” are used interchangeably herein and refer to the introduction of the cells disclosed herein to target tissue. As mentioned, the cells can be derived from the recipient or from an allogeneic, semi-allogeneic or xenogeneic donor.

The disclosed miR-155-3p or miR-155-5p biased DA neural cells can be injected systemically into the circulation, administered intrathecally or grafted into the central nervous system, the spinal cord or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the brain parenchyma at the time of transplantation.

Intraparenchymal transplantation can be performed using two approaches: (i) injection of cells into the host brain parenchyma or (ii) preparing a cavity by surgical means to expose the host brain parenchyma and then depositing the graft into the cavity.

Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a micro syringe to be inserted. The micro syringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The disclosed miR-155-3p or miR-155-5p biased DA neural cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the disclosed miR-155-3p or miR-155-5p biased DA neural cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions.

Multiple grafts may consist of a mixture of cell types, and/or a mixture of transgenes inserted into the cells. Preferably from approximately 104 to approximately 109 cells are introduced per graft. Cells can be administered concomitantly to different locations such as combined administration intrathecally and intravenously to maximize the chance of targeting into affected areas.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

Intranasal administration of the disclosed miR-155-3p or miR-155-5p biased DA neural cells is also contemplated.

The use of differentiated modified miR-155-3p or miR-155-5p pluripotent cells may be also indicated for treatment of traumatic lesions of the nervous system including spinal cord injury and also for treatment of stroke caused by bleeding or thrombosis or embolism because of the need to induce neurogenesis and provide survival factors to minimize insult to damaged neurons.

The disclosed miR-155-3p or miR-155-5p biased DA neural cells may be useful for neurological diseases including, but not limited to amyotrophic lateral sclerosis (ALS), primary lateral sclerosis (PLS), pseudobulbar palsy and progressive bulbar palsy.

Since non-autologous cells may induce an immune reaction when administered to the body several approaches have been developed to reduce the likelihood of rejection of non-autologous cells. Furthermore, since diseases such as multiple sclerosis are inflammatory based diseases, the problem of immune reaction is exacerbated. These include either administration of cells to privileged sites, or alternatively, suppressing the recipient's immune system, providing anti-inflammatory treatment which may be indicated to control autoimmune disorders to start with and/or encapsulating the non-autologous/semi-autologous cells in immunoisolating, semipermeable membranes before transplantation.

As mentioned herein above, the present inventors also propose use of cord and placenta-derived pluripotent cells that express very low levels of MHCII molecules and therefore limit immune response.

The following experiments may be performed to confirm the potential use of newborn's pluripotent cells isolated from the cord I placenta for treatment of neurological disorders:
1) Differentiated pluripotent cells (to various neural cells or neural progenitor cells) may serve as stimulators in one-way mixed lymphocyte culture with allogeneic T-cells and proliferative responses in comparison with T cells responding against allogeneic lymphocytes isolated from the same donor may be evaluated by 3H Thymidine uptake to document hyporesponsiveness.
2) Differentiated pluripotent cells may be added/co-cultured to one-way mixed lymphocyte cultures and to cell cultures with T cell mitogens (phytohemmaglutinin and concanavalin A) to confirm the immunosuppressive effects on proliferative responses mediated by T cells.
3) Cord and placenta cells cultured from Brown Norway rats (unmodified and differentiated), may be enriched for pluripotent cells and these cells may be infused into Lewis rats with induced experimental autoimmune encephalomyelitis (EAE). Alternatively, cord and placenta cells cultured from BALB/c mice, (BALB/cxC57BL/6)F1 or xenogeneic cells from Brown Norway rats (unmodified and differentiated), may be enriched for pluripotent cells and these cells may be infused into C57BL/6 or SJL/j recipients with induced experimental autoimmune encephalomyelitis (EAE). The clinical effects against paralysis may be investigated to evaluate the therapeutic effects of xenogeneic, fully MHC mismatched or haploidentically mismatched pluripotent cells. Such experiments may provide the basis for treatment of patients with a genetic disorder or genetically proned disorder with family member's haploidentical pluripotent cells.
4) pluripotent cells cultured from cord and placenta may be transfused with pre-miR labeled with GFP or RFP, which will allow the inventors to follow the migration and persistence of these cells in the brain of C57BL/6 recipients with induced EAE. The clinical effects of labeled MHC mismatched differentiated pluripotent cells may be evaluated by monitoring signs of disease, paralysis and histopathology. The migration and localization of such cells may be also monitored by using fluorescent cells from genetically transduced GFP “green” or Red2 “red” donors.

As mentioned, the present invention also contemplates encapsulation techniques to minimize an immune response.

### Encapsulation Techniques

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42:29-64).

Methods of preparing microcapsules are known in the arts and include for example those disclosed by Lu M Z, et al., Cell encapsulation with alginate and alpha phenoxycinnamylidene-acetylated poly(allylamine). Biotechnol Bioeng. 2000, 70:479-83, Chang T M and Prakash S. Procedures for microencapsulation of enzymes, cells and genetically engineered microorganisms. Mol. Biotechnol. 2001, 17:249-60, and Lu M Z, et al., A novel cell encapsulation method using photosensitive poly(allylamine alpha-cyanocinnamylideneacetate). J. Microencapsul. 2000, 17:245-51.

For example, microcapsules are prepared by complexing modified collagen with a per-polymer shell of 2-hydroxyethyl methylacrylate (HEMA), methacrylic acid (MAA) and methyl methacrylate (MMA), resulting in a capsule thickness of 2-5 um. Such microcapsules can be further encapsulated with additional 2-5 um per-polymer shells in order to impart a negatively charged smooth surface and to minimize plasma protein absorption (Chia, S. M. et al. Multi-layered microcapsules for cell encapsulation Biomaterials. 2002 23:849-56).

Other microcapsules are based on alginate, a marine polysaccharide (Sambanis, A. Encapsulated islets in diabetes treatment. Diabetes Technol. Ther. 2003, 5:665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 um (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10:6-9; Desai, T. A. Microfabrication technology for pancreatic cell encapsulation. Expert Opin Biol Ther. 2002, 2:633-46).

### Immunosuppressive Agents

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE™), etanercept, TNF alpha blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium, salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells can be administered either per se or, preferably as a part of a pharmaceutical composition that further comprises a pharmaceutically acceptable carrier.

Techniques for formulation and administration of drugs may be found in “Remington's Pharmaceutical Sciences,” Mack Publishing Co., Easton, Pa., latest edition, which is incorporated herein by reference.

### Administration

Suitable routes of administration include direct administration into the circulation (intravenously or intra-arterial), into the spinal fluid or into the tissue or organ of interest. Thus, for example the cells may be administered directly into the brain.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, animal models of demyelinating diseases include shiverer (shi/shi, MBP deleted) mouse, MD rats (PLP deficiency), Jimpy mouse (PLP mutation), dog shaking pup (PLP mutation), twitcher mouse (galactosylceramidase defect, as in human Krabbe disease), trembler mouse (PMP-22 deficiency). Virus induced demyelination model comprise use if Theiler's virus and mouse hepatitis virus. Autoimmune EAE is a possible model for multiple sclerosis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, a multiple sclerosis patient can be monitored symptomatically for improved motor functions indicating positive response to treatment.

For injection, the active ingredients of the pharmaceutical composition may be formulated in aqueous solutions, preferably in physiologically compatible buffers such as Hank's solution, Ringer's solution, or physiological salt buffer.

Dosage amount and interval may be adjusted individually to levels of the active ingredient which are sufficient to effectively treat the brain disease/disorder. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated multiple sclerosis patient will be administered with an amount of cells which is sufficient to alleviate the symptoms of the disease, based on the monitoring indications.

The miR-155-3p or miR-155-5p biased DA neural cells of the present invention may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA.

### Kits

Also provided are kits for administration of the compositions and carrying out the diagnostic methods comprising the composition that may include an appropriate dosage amount. Kits may further comprise suitable packaging and/or instructions for use of the compositions and/or diagnostic methods. Kits may also comprise a means for the delivery of the at least one compositions, and syringe for injection.

Additionally, the kits can contain the composition and reagents to prepare a miR-155-3p or miR-155-5p biased DA neural cell composition for administration. The kit may contain a device for administration or for dispensing the compositions, including, but not limited to, syringe, pipette, or transdermal patch.

The kits may include other therapeutic compounds for use in conjunction with the compounds described herein and as such, the methods as disclosed herein can contain other appropriate therapeutic compounds or agents. These compounds can be provided in a separate form or mixed with the miR-155-3p or miR-155-5p biased DA neural cell compositions of the present disclosure. The kits will include appropriate instructions for preparation and administration of the miR-155-3p or miR-155-5p biased DA neural cell composition, side effects of the compositions, and any other relevant information. The instructions can be in any suitable format, including, but not limited to, printed matter, videotape, computer readable disk, or optical disc.

Kits may also be provided that contain sufficient dosages of the compounds or composition to provide effective treatment for an individual for an extended period, such as a week, 2 weeks, 3, weeks, 4 weeks, 6 weeks, or 8 weeks or more.

The following examples are intended to illustrate, and not limit, the disclosed herein. For example, while the examples are noted to be for the isolation, purification and use of exosome compositions for the treatment of a fibrotic or liver disease or an associated disorder, the methods and compositions can be modified for the treatment of other neurologic diseases as noted herein.

### Examples

The above disclosure can be further understood by the following prophetic examples.

### Example 1: Directed Editing of BIC Hsa-miR-155

The principal pro-inflammatory network is activated by miR-155-5p

Hypothesis: driving miR-155-5p >>miR-155-3p will enhance inflammatory activity

Hypothesis: driving miR-155-3p >>miR-155-5p will enhance anti-inflammatory activity

One miRNA, miR-155, maps within, and is processed from, an exon of the noncoding RNA known as bic, its primary miRNA precursor. bic/miR-155 shows greatly increased expression in activated B and T cells, as well as in activated macrophages and dendritic cells (DCs). Overexpression of bic/miR-155 has been reported in B cell lymphomas and solid tumors, and transgenic miR-155 mice have also been shown to develop B cell malignancies in vivo, indicating that the locus may also be linked to cancer.

Introduce into the BIC Locus Edited Versions of miR-155 to Enhance-5p and miR-155-3p Output

Lipofectamine® 2000 reagent (Invitrogen, Life Technologies, USA) can be used to deliver single-stranded RNA molecules (mirVana™ miRNA Mimics and Inhibitors; Ambion, Life Technologies, USA) into iPSCs as per manufacturer's instructions. Briefly, cells were seeded at 2×105 cells per well of a 6-well plate and transfected with 30 nM of mimic (miR-155-5p, the Negative Control #1, miR-155-3p, the Negative Control #2) or modified hsa-miR-155 (miR-155-5p: Seq Id No 6 SEQ ID No 6; miR-155-3p: SEQ ID No 7; and miR-155-stem loop: SEQ ID No 8). After transfection, cells can be added to pre-warmed media and immediately placed in an incubator at 37° C. in a 5% CO₂ atmosphere. Cells can be harvested at 24 and 48 h after transfection for functional assays.

Confirm that editing changes in hsa-miR-155 in iPSCs; miR-155-3p and miR-155-3p and transcriptomes for wildtype and the miR-155-3p and miR-155-3p enhanced versions

Characterize differentiation into RNDP-001 to assess potential changes in phenotype/secretome

### Example 2: Differentiation of Modified Pluripotent Cells to Modified Day 17 DA Neurons

Midbrain neuronal differentiation of modified human induced pluripotent stem cell (iPS) cell lines expanded on VTN-TN in Essential 8 medium was performed with small molecule and growth factor induction using a variety of differentiation media compositions and schedules as detailed in Tables 1-5. Generally, the modified iPS cells were cultured in D1 DA Neuron Induction Medium on Day 1, D2 Neuron Induction Medium on Day 2, and D3-D4 DA Induction Medium on Day 3 and 4. On Day 5, the modified cells were dissociated with TrypLE for 15 minutes and collected in DA Quench Medium before transferring the cells to a spinner flask suspension culture to form aggregates in D5 DA Neuron Aggregate Formation Medium.

On Day 6, the modified aggregates were settled, about 66% of the medium was removed, and the aggregates were fed DA Neuron Induction Medium. On Days 7-16, the aggregates were fed daily with DA Neuron Aggregate Maintenance Medium, and the medium was changed on Day 11 through 16. On Day 17, modified aggregates were dissociated to a single-cell suspension with TrypLE and plated onto Matrigel in D17 DA Neuron Aggregate Plating Medium.

The differentiation technique can be further understood by reference to Table 1 above.

Example 3: Administration of Each of the Edited iPSC Differentiated to D17 DA Neurons

Administer each of the edited iPSC-derived RNDP-001 (unedited, miR-155-3p (Seq ID No 3) and miR-155-3p edited (Seq ID No 7) miR-155-5p (Seq ID No 1) and miR-155-5p edited (Seq ID No 6)) into 8 OHDA nude rat hemiparkinsonian model and measure behavior (baseline and +D-amphetamine) relating to inflammation, image inflammatory mass/region.

Harvest tissue for ICC, flow cytometry and RNAseq

### Example 4: Non-Regulated Transfected Hsa-miR-155

Transient transfection of miR-155 constructs (wildtype, miR-155-3p enhanced and miR-155-3p enhanced)

NLP transfected miR-155 iPSCs

Lipid nanoparticles (LNPs) have been developed and used extensively as nonviral (or synthetic) vectors to treat genetic and acquired disorders in gene therapy. LNPs are safer than viral vectors due to the absence of immunogenic viral proteins.

RNAseq to determine change in transcriptome

Example 5: Administration of Each of the Edited iPSC Differentiated to D17 DA Neurons

Administer NLP-transfected RNDP-001 in nude rats+/−6OHDA hemiparkinsonism with three constructs and measure neuroinflammatory markers, animal weight and survival

Harvest tissue for ICC, flow cytometry and RNAseq

Example 8: Use of miR-155-3p Biased D17 DA Neural Cells to Treat Spinal Cord Injury

It is hypothesized that miR-155-3p biased D17 DA neural cells can be used to treat nerve, and specifically spinal cord, injury. Wild-type rats would undergo spinal cord perfusion injury by blocking the abdominal aorta below the left renal artery for 15 minutes. The injured rats can then be treated with PBS or miR-155-3p biased D17 DA neural cells (1×10^7 cells) injected at the L5-L6 segment of the spine. Four days later lower limb movement in the rats will be evaluated using the Basso, Beattie and Bresnahan (BBB) locomotor scale method. Uninjured rats can also be evaluated as a control. The BBB scale is a well-established and discriminating method for measuring behavioral outcome and for evaluating treatments after spinal cord injury. The scale ranges from zero to 21, with a higher score indicating superior movement. The scoring can be summarized by the following breakdown:
0-7: Isolated joint movements with little or no hindlimb movement.
8-13: Intervals of uncoordinated stepping.
14-21: Forelimb and hindlimb coordination.

Uninjured mice should have a nearly perfect score of 20.6 on the BBB scale, whereas control injured mice treated with only PBS should score in the lowest category with an average score of around 4. Mice treated with miR-155-3p biased D17 DA neural cells should show a strong improvement in locomotion, with an average score of around 12, which is the upper half of the middle category, and should be capable of uncoordinated stepping.

What is claimed is:

1. A DA neuronal cell in which the level of miR-155-3p (CAUAAAGUAGAAAGCACUACU) or a variant thereof is increased or decreased compared to wildtype or native unmodified cells.

2. The DA neuronal cell of claim 1, wherein said variant comprises or consists of a nucleotide sequence with SEQ ID No 7.

3. The DA neuronal cell of claim 1, wherein said variant comprises or consists of a nucleotide sequence in which all of the nucleotides corresponding to nucleotides 2 to 22 of said SEQ ID No 3 are retained.

4. The DA neuronal cells of claim 1, wherein said DA neuronal cells are genetically modified or genetically engineered.

5. The DA neuronal cells of claim 1, wherein SEQ ID NO 8 or a variant thereof is integrated into the DA neuronal cells to increase the level of miR-155-3p (CAUAAAGUAGAAAGCACUACU) or a variant thereof.

6. The DA neuronal cells of claim 1, wherein additional copies of miR-155-3p (CAUAAAGUAGAAAGCACUACU) or a variant thereof are inserted into the DA neuronal cells to increase the level of miR-155-3p (CAUAAAGUAGAAAGCACUACU) or a variant thereof.

7. The DA neuronal cells of claim 1, wherein the endogenous genomic sequence encoding miR-155-5p (UUAAUGCUAAUCGUGAUAGGGGUU) or a variant thereof is deleted or mutated in said DA neuronal cells to decrease the level of miR-155-5p (UUAAUGCUAAUCGUGAUAGGGGUU) or a variant thereof.

8. The DA neuronal cells of claim 1, wherein said DA neuronal cells are human cells.

9. The DA neuronal cells of claim 1, for use in a therapy.

10. The DA neuronal cell of claim 9, wherein the level of miR-155-3p (CAUAAAGUAGAAAGCACUACU) or a variant thereof is increased, for use in the treatment of Parkinsonian disorders.

11. The DA neuronal cell of claim 9, wherein the level of miR-155-5p (UUAAUGCUAAUCGUGAUAGGGGUU) or a variant thereof is decreased, for use in the treatment of Parkinsonian disorders.

12. The DA neuronal cell of claim 9, wherein said therapy is an autologous therapy.

13. The DA neuronal cell of claim 9, wherein said therapy is an allogenic therapy.

14. (canceled)

15. (canceled)

16. (canceled)

17. (canceled)

18. (canceled)

19. (canceled)

20. (canceled)

21. (canceled)

22. (canceled)

24. (canceled)

25. (canceled)

26. The DA neuronal cell of claim 1, The DA neuronal cell of claim 27, wherein the level of miR-155-3p (CAUAAAGUAGAAAGCACUACU) or a variant thereof is increased or decreased compared to wildtype differentiated cell populations.

27. The DA neuronal cell of claim 1, wherein the level of miR-155-3p (CAUAAAGUAGAAAGCACUACU) or a variant thereof is increased compared to miR-155-5p (UUAAUGCUAAUCGUGAUAGGGGUU) or a variant thereof.

28. The DA neuronal cell of claim 1, wherein the level of miR-155-5p (UUAAUGCUAAUCGUGAUAGGGGUU) or a variant thereof is increased compared to miR-155-3p (CAUAAAGUAGAAAGCACUACU) or a variant thereof.

29. The DA neuronal cell of claim 1, wherein the level of miR-155-5p (UUAAUGCUAAUCGUGAUAGGGGUU) or a variant thereof is increased compared to wildtype DA neuronal cells.

30. The DA neuronal cell of claim 1, wherein the DA neuronal cell expresses forkhead box protein A2 (FOXA2), LIM homeobox transcription factor 1 (LMX1) and tyrosine hydroxylase (TH).

31. A DA neuronal cell in which the level of miR-155-3p (CAUAAAGUAGAAAGCACUACU) or a variant thereof is increased compared to wildtype DA neuronal cells and wherein the endogenous genomic sequence encoding miR-155-5p (UUAAUGCUAAUCGUGAUAGGGGUU) or a variant thereof is deleted or mutated in said DA neuronal cells.

32. A DA neuronal cell in which the level of miR-155-3p (CAUAAAGUAGAAAGCACUACU) or a variant thereof is increased compared to wildtype DA neuronal cells and compared to miR-155-5p (UUAAUGCUAAUCGUGAUAGGGGUU) or a variant thereof.


SEQUENCE LISTING (22 sequences)

SEQ ID NO: 1 - RNA, 24 nt, synthetic construct
  source 1..24: mol_type = other RNA; note = human hsa-miR-155-5p MIMAT0000646
ttaatgctaatcgtgataggggtt

SEQ ID NO: 2 - RNA, 24 nt, synthetic construct
  source 1..24: mol_type = other RNA; note = mouse miR-155-5p MIMAT0000646
ttaatgctaattgtgataggggtt

SEQ ID NO: 3 - RNA, 22 nt, synthetic construct
  source 1..22: mol_type = other RNA; note = human hsa-miR-155-3p MIMAT0004658
ctcctacatattagcattaaca

SEQ ID NO: 4 - RNA, 22 nt, synthetic construct
  source 1..22: mol_type = other RNA; note = mouse -miR-155-3p MIMAT0004658
ctcctacctgttagcattaaca

SEQ ID NO: 5 - RNA, 65 nt, synthetic construct
  source 1..65: mol_type = other RNA; note = hsa-miR-155 stem loop MI0000681
ctgttaatgctaatcgtgataggggtttttgcctccaactgactcctacatattagcattaacag

SEQ ID NO: 6 - RNA, 24 nt, synthetic construct
  source 1..24: mol_type = other RNA; note = modified hsa-miR-155-5p
ggaatgctaatcgtgataggggtt

SEQ ID NO: 7 - RNA, 22 nt, synthetic construct
  source 1..22: mol_type = other RNA; note = modified hsa-miR-155-3p
ttcctacatattagcattaaca

SEQ ID NO: 8 - RNA, 65 nt, synthetic construct
  source 1..65: mol_type = other RNA; note = modified hsa-miR-155 stem loop
ctgttaatgctaatcgtgatagggatttttgcctccaactgattcctacatattagcattaacag

SEQ ID NO: 9 - RNA, 20 nt, synthetic construct
  source 1..20: mol_type = other RNA; note = miR-155-3p isomiR
ctcctacatattagcatcca

SEQ ID NO: 10 - RNA, 20 nt, synthetic construct
  source 1..20: mol_type = other RNA; note = miR-155-3p isomiR
tcctacatattagcattaac

SEQ ID NO: 11 - RNA, 21 nt, synthetic construct
  source 1..21: mol_type = other RNA; note = miR-155-3p isomiR
tcctacatattagcattaaca

SEQ ID NO: 12 - RNA, 19 nt, synthetic construct
  source 1..19: mol_type = other RNA; note = miR-155-3p isomiR
ctcctacatattagcatca

SEQ ID NO: 13 - RNA, 19 nt, synthetic construct
  source 1..19: mol_type = other RNA; note = miR-155-3p isomiR
tcctacatattagcattaa

SEQ ID NO: 14 - RNA, 19 nt, synthetic construct
  source 1..19: mol_type = other RNA; note = miR-155-3p isomiR
ctcctacatattagcatta

SEQ ID NO: 15 - RNA, 17 nt, synthetic construct
  source 1..17: mol_type = other RNA; note = miR-155-3p isomiR
actcctacatattagca

SEQ ID NO: 16 - RNA, 18 nt, synthetic construct
  source 1..18: mol_type = other RNA; note = miR-155-3p isomiR
gactcctacatattagca

SEQ ID NO: 17 - RNA, 20 nt, synthetic construct
  source 1..20: mol_type = other RNA; note = miR-155-3p isomiR
ctcctacatattagcataac

SEQ ID NO: 18 - RNA, 20 nt, synthetic construct
  source 1..20: mol_type = other RNA; note = miR-155-3p isomiR
actcctacatattagcatta

SEQ ID NO: 19 - RNA, 19 nt, synthetic construct
  source 1..19: mol_type = other RNA; note = miR-155-3p isomiR
ctcctacatattagcattc

SEQ ID NO: 20 - RNA, 18 nt, synthetic construct
  source 1..18: mol_type = other RNA; note = miR-155-3p isomiR
tacatattagcattaaca

SEQ ID NO: 21 - RNA, 18 nt, synthetic construct
  source 1..18: mol_type = other RNA; note = miR-155-3p isomiR
ctcctacatattagcatt

SEQ ID NO: 22 - RNA, 21 nt, synthetic construct
  source 1..21: mol_type = other RNA; note = miR-155-3p
cataaagtagaaagcactact